(19) United States
(12) Patent Application Publication
(10) Pub. No.: US 2010/0291058 A1
(43) Pub. Date: Nov. 18, 2010

(54) Sealants for Skin and Other Tissues

(75) Inventors: Gary L. Bowlin, Mechanicsville, VA (US); David G. Simpson, Mechanicsville, VA (US); Gary E. Wnek, Midlothian, VA (US); Marcus E. Carr, JR., Midlothian, TX (US); Peter J. Stevens, N. Richland Hills, TX (US); Gary Cadd, Grapevine, TX (US); I. Kelman Cohen, Richmond, VA (US)

Correspondence Address: FOLEY HOAG, LLP;PATENT GROUP, WORLD TRADE CENTER WEST, 155 SEAPORT BLVD, BOSTON, MA, 02110, US

(73) Assignees: Virginia Commonwealth University, Richmond, VA (US); Nanomatrix, Inc., Addison, TX (US)

(21) Appl. No.: 12/758,360

(22) Filed: Apr. 12, 2010

Related U.S. Application Data
(63) Continuation of application No. 10/588,344, filed on Jan. 8, 2007, now abandoned.
(60) Provisional application No. 60/416,026, filed on Oct. 4, 2002.
(60) Provisional application No. 60/425,949, filed on Nov. 13, 2002.

(51) Int. Cl. A61K 38/14; B32B 5/02; C07K 14/75; A61K 38/48; A61K 38/45; A61P 17/00; A61P 17/02; C25D 9/02

(52) U.S. Cl. 424/94.5; 428/401; 530/382; 514/15.3; 424/94.64; 205/316

(57) ABSTRACT

The present invention relates to sealants for skin and other tissues. The sealants include an electroprocessed material. The sealants may contain more than one electroprocessed materials and may contain additional substances. The invention further relates to methods of making and using such sealants.

### FIELD OF THE INVENTION

The present invention relates to sealants for skin and other tissues and to methods of making and using such sealants. The sealants include an electroprocessed material. The sealants may contain more than one electroprocessed material and may contain additional substances.

### BACKGROUND OF THE INVENTION

A continuing need exists for sealants useful to repair, seal, adhere, or connect tissues, to have a hemostatic effect, or both. Depending on the application of the sealant, desirable features of such sealants can include, but are not limited to: causing hemostasis at a desired rate, including by formation of clots; the ability to be formed into a variety of shapes, including complex shapes; structural strength and mechanical integrity (for example, sufficient integrity to withstand application of pressure to a sealant when used as a bandage). Many sealants involve the use of fibrin, a component of natural blood clots. Many sealants use the combination of fibrinogen and thrombin to form fibrin. In aqueous environments, thrombin causes conversion of fibrinogen to fibrin. To avoid premature formation of fibrin, many sealants must be formed by combining elements immediately before use, and cannot be stored together. In addition, many sealants have little structural strength. In fact, many have a gel consistency and thus do not hold their shape in response to physical forces such as the application of pressure or vigorous flow of blood or other fluids from a wound or opening. Accordingly, there is a need in the art for sealants that have these features.

### SUMMARY OF THE INVENTION

The present invention includes tissue sealant compositions. The compositions are used, for example, as hemostatic agents or agents that can prevent, reduce, or eliminate the flow of a fluid. The compositions are also used as adhesives for attaching tissues or structures of an organism to each other or to other objects, as scaffoldings for structural support for tissue or organs, and as sealants that can close, cover, obstruct, fill, or seal any type of leak, wound, ulcer, injury, opening, hole, or cavity. The sealants can be in the form of a matrix and can serve as matrices for tissue growth.

One component of the compositions of the present invention is an electroprocessed material. The electroprocessed material of the present invention can include natural materials, synthetic materials, or combinations thereof. Some especially preferred natural materials include the product that results from electroprocessing collagen, fibrin, fibrinogen, thrombin, or fibronectin, and combinations thereof. In many desirable embodiments, the electroprocessed materials are combined with one or more substances. The word “substance” in the present invention is used in its broadest definition and includes any type or size of molecules, cells, objects or combinations thereof. In a preferred embodiment, a tissue sealant containing the product that results from electroprocessing collagen, fibrinogen, fibronectin, thrombin, synthetic polymers, or combinations thereof, contains other substances to assist coagulation or to provide other biological responses. Examples include coagulation factors, other proteins and factors in the coagulation cascade, substances that regulate or enhance healing, and chemicals that inhibit fibrinolysis or otherwise inhibit breaking down of a clot.

The stability of the electroprocessed sealant compositions of the present invention allows for long term storage between formation and use. Electroprocessed materials in some embodiments are substantially dry, thus allowing the products of electroprocessing fibrinogen, thrombin, and other factors in the coagulation cascade to be combined and stored together in a dry state without the risk of premature formation of a clotted composition that cannot be used. This is advantageous as compared to other sealants in which components must be stored separately and mixed immediately before use. Some embodiments have hemostatic properties. Embodiments exist that have varying speeds of hemostasis, thus allowing preparation of compositions that cause hemostasis at a desired speed. Thus, embodiments can be tailored to function more effectively, for example, with oozing wounds or with rapidly hemorrhaging wounds. In many embodiments, the use of the sealants of the present invention helps reduce the degree of scar formation in the location of use. In some embodiments, the compositions form a matrix, preferably a matrix similar to an extracellular matrix. In some embodiments, the sealant matrix has a pore size that is small enough to be impermeable to red blood cells, thus preventing leaking. In some embodiments, the sealant matrix has a pore size that is small enough to reduce or to eliminate evaporative water loss from a wound. Alternatively, a portion of the sealant, such as the outermost portion, has small pore size or is a film having essentially no pores to reduce or to eliminate evaporative water loss. Some embodiments are tailored to allow or to promote infiltration of the matrix with cells. Electroprocessed sealants have the further advantage in some embodiments of having greater structural strength than many known sealants, and of retaining that structural strength after application or implantation. As such, they can be subjected to physical pressure and can withstand vigorous flows of blood and other fluids without being washed away. In some embodiments, however, the sealants are highly labile such that they dissolve or otherwise disintegrate rapidly upon contact with aqueous fluids. The sealant matrices can also have varying degrees of elasticity. It is also possible to prepare combined electroprocessed compositions containing a variety of materials.

The present invention also provides electroprocessed sealant materials or extracellular matrices having a predetermined shape, as well as methods for making those shaped materials. Virtually any shape is possible. Some preferred examples include a cylindrical shape, a flattened oval shape, a sphere, a fluff or batt, a rectangular envelope shape, a sheet, a ribbon, a cylinder, a sleeve for placing around a vessel or duct, a dural patch, a nerve guide, skin or muscle patch, fascial sheath, vertebral disc, articular cartilage, knee meniscus, ligament, tendon, or a vascular graft for subsequent use in vivo.

The invention further includes methods of making the sealants of the present invention. The method includes electroprocessing one or more materials. The method can further include combining the material with one or more substances. Many embodiments of the present invention involve means for manipulating the pattern or distribution of electroprocessed material and/or substances within an electroprocessed material. For example, a target can also be specifically charged or grounded along a preselected pattern so that electroprocessed materials streaming toward the target are directed into specific directions or distributions on the target or on a substrate. The electric field can be controlled by a microprocessor to create a matrix having a desired geometry. Other features that allow establishment of such a pattern include, but are not limited to, the ability to deposit multiple layers of the same or of different materials, combining different electroprocessing methods, the use of multiple orifices with different contents for electroprocessing, and the existence of numerous methods for combining substances with the materials. The compositions may then be further processed, for example by shaping, crosslinking, or combining with substances. Substances may be combined with electroprocessed materials before, during, or after electroprocessing. For example, substances can be applied to the electroprocessed material after formation, for example by soaking the electroprocessed material in the substance or a solution containing the substance or by spraying the solution or substance onto the electroprocessed material. Electroprocessed sealants containing cells can be placed into a culture to enhance the cell growth. Cells can also be placed in a lumen or space within a construct, or implanted adjacent to the implant to facilitate growth.

The electroprocessed tissue sealants of the present invention have many uses and methods of using the sealants are also within the present invention. They are used as hemostatic agents to stop bleeding at the site of a wound or injury or at the site at which surgery has occurred or will occur. Tissue sealants are also used to create an obstruction or reinforcement for an obstruction to a leak of any material to or from any location in the body of an organism. The sealants are also used for a variety of other functions associated with attachment, connection, providing structural support, or providing a scaffolding for cells, tissue, or organs. Other uses include, but are not limited to, use in the manufacture of engineered tissue and organs. The sealants may be applied in any form. Some preferred forms include as a sheet or strip for direct application, a component of a bandage or gauze, and a powder or fluff that may be packed or sprinkled onto or into a location of a wound or injury. In some embodiments, the sealants are combined with water absorbent materials to provide water absorbency. Another use of the electroprocessed compositions of the present invention is the delivery of one or more substances to a desired location, including delivery of pharmaceuticals to a location in an organism.

Accordingly, it is an object of the present invention to overcome the foregoing limitations and drawbacks by providing tissue sealant compositions.

It is further an object of the present invention to provide tissue sealant compositions that comprise one or more electroprocessed materials.

It is further an object of the present invention to provide compositions that have a hemostatic effect.

It is further an object of the present invention to provide adhesives for attaching tissues, organs or structures of an organism to each other or to other objects.

It is further an object of the present invention to provide scaffoldings for structural support of tissue or organs.

It is further an object of the present invention to provide sealants that can cover, obstruct, fill or seal one or more types of wound, ulcer, injury, hole, leak, cavity, enclosure, or opening in any tissue, organ, or part of any organism.

It is further an object of the present invention to provide compositions that can block, prevent, reduce, or eliminate the flow of any fluid, liquid or gas.

It is further an object of the present invention to provide tissue sealant compositions that can be stored in a dry form.

It is further an object of the present invention to provide tissue sealant compositions that can be stored at room temperature.

It is further an object of the invention to provide tissue sealant compositions that can be stored as a single component.

Another object of the present invention is to provide compositions comprising electroprocessed materials and non-electroprocessed materials.

A further object of the present invention is to provide compositions comprising electroprocessed materials and cells, molecules, objects, or combinations thereof.

Still another object of the present invention is to provide methods of making the compositions of the present invention.

It is further an object of the present invention to provide methods of making constructs comprising the compositions of the present invention.

It is further an object of the present invention to provide methods of using the compositions of the present invention.

Another object of the present invention is to provide methods of substance delivery.

Another object of the present invention is to provide compositions for use in substance delivery.

It is further an object of the present invention to provide methods for cell and tissue culture.

These and other objects, features and advantages of the present invention will become apparent after a review of the following detailed description of the disclosed embodiments, the drawings, and the claims.

### BRIEF DESCRIPTION OF THE FIGURES

FIG. 1 is a scanning electron micrograph illustrating a higher magnification view of an electrospun matrix prepared by electrospinning a solution of human fibrinogen. The average fiber sizes in this matrix were around 100-200 nm.

FIG. 2 is a scanning electron micrograph illustrating an electrospun matrix prepared by electrospinning a solution of bovine fibrinogen and bovine collagen in HFP/MEM (external surface view). The fibers produced were 1 micron or less in diameter. The natural polymer concentration was high to start in solution, thus the large fiber diameters were expected.

FIG. 3 is a scanning electron micrograph illustrating an electrospun matrix prepared by electrospinning a solution of bovine fibrinogen and bovine collagen in HFP/MEM (external surface view).

FIG. 4 is a scanning electron micrograph illustrating an electrospun matrix prepared by electrospinning a solution of bovine fibrinogen and bovine collagen in HFP/MEM (external surface view) on a 4 mm ID tubular scaffold. The fibers are highly aligned due to the rotational speed of the mandrel during processing.

FIG. 5 is a schematic of the dog-bone template used for the cutting of samples for bulk material testing.

FIG. 6 is a photograph of a mat an electrospun matrix prepared by electrospinning a solution containing ⅙^{th} weight fibrinogen by volume solution. The mat has a mass of 0.0778 g, average thickness of 0.0263 in (0.6680 mm), and length and width of 10 cm by 10 cm.

FIG. 7 depicts four scanning electron micrographs of compositions. The images show an electrospun matrix prepared by electrospinning a solution of Collagen (A), an electrospun matrix prepared by electrospinning a solution of VITROGEN (B), an electrospun matrix prepared by electrospinning a solution of gelatin (C) and INTEGRA (D).

FIG. 8 depicts micrographs of full thickness dermal wounds in the guinea pig 7 days after application of various structures to the wounds. The images show wounds having structures of INTEGRA (A), electrospun collagen (B) electrospun VITROGEN, (C) and electrospun gelatin (D). In each image the arrows to the right of the images indicate the margin of the wound and the site where the epithelial tongue will develop. Small black “dots” along the surface of C are silver grains. The silver is present at irregular intervals in all implants due to use of a silver-impregnated dressing placed over the electrospun materials and the INTEGRA.

FIG. 9 depicts micrographs of full thickness dermal wounds in the guinea pig 14 days after application of various structures to the wounds. The images show wounds having structures of INTEGRA (A), electrospun collagen (B) electrospun VITROGEN, (C) and electrospun gelatin (D).

FIG. 10 depicts micrographs of full thickness dermal wounds in the guinea pig 7 days after application of various structures to the wounds. Images illustrate the utility of using an aligned matrix of electrospun collagen to accelerate dermal fibroblast alignment.

FIG. 11 is a schematic drawing of an embodiment of an electroprocessing device including the electroprocessing equipment and a substrate.

FIG. 12 is a schematic drawing of an embodiment of an electroprocessing device including the electroprocessing equipment and a substrate.

FIG. 13 is a schematic drawing of an embodiment of an electroprocessing device including the electroprocessing equipment and a substrate.

FIG. 14 is a graph depicting fiber diameters resulting from the electrospinning various solutions containing bovine fibrinogen in differing concentrations in HFP/MEM with all other parameters constant. The graph illustrates the linear relationship (R2=0.98) between concentration and fiber diameters composing the structures produced.

### DETAILED DESCRIPTION OF THE PREFERRED EMBODIMENTS

The present invention includes tissue sealant compositions. The compositions are used, for example, as hemostatic agents or agents that can prevent, reduce, or eliminate the flow of a fluid or can assist in repair of an injury or reinforcement of a tissue. The compositions are also used as adhesives for attaching tissues or structures of an organism to each other or to other objects, as scaffoldings for structural support for tissue or organs, and as sealants that can close, cover, obstruct, fill, or seal any type of leak, wound, ulcer, injury, opening, hole, or cavity. The sealants can be in the form of a matrix and can serve as matrices for tissue growth. The sealant compositions comprise electroprocessed materials. In some embodiments, the sealant is an electroprocessed collagen or electroprocessed fibrinogen. In some embodiments, the electroprocessed material comprises fibers having an average diameter between about 50 nm and about 10 μm. In some embodiments, the fibers have a repeating banding pattern along the axis of the fiber characteristic of natural fibers. In some embodiments, the sealants further comprise one or more substances. Examples of substances include, but are not limited to, thrombin, aprotinin, Factor XIII, calcium chloride, hydroxyapatite, a fibrinolytic inhibitor, a fibrinolytic agent, fibronectin, or a combination thereof. The invention also includes methods of use of the compositions of the present invention and methods of providing the effect of a sealant. The invention also comprises methods of making the sealants of the present invention.

### DEFINITIONS

The terms “sealant” and “tissue sealant” shall be given their broadest possible meaning and shall include, but not be limited to, any substance, composition, material or object that can form, reinforce, or strengthen any type of bond, attachment, seal, connection, communication, or other physical association between any tissue, organ, structure or other part of an organism and any other substance, composition, or object. The “other substance, composition, or object” can be any type of substance, cell, composition, or object, or combination or composites thereof including, but not limited to: one or more portions of the same tissue, organ, structure or part of the organism; one or more different tissues, organs, structures, or parts of the same organism; one or more other organisms; one or more tissues, cells, organs, structures, or parts of one or more other organisms; one or more synthetic or inanimate compositions, substances, or objects (e.g. medical devices, prosthetics, implants, carriers for delivery of a pharmaceutical, neutraceutical, or other substance), or portions thereof; and any combination or composite of one or more of the foregoing. The terms “sealant” and “tissue sealant” also include materials and substances that can serve as glues or adhesives. The terms “sealant” and “tissue sealant” also include any substance, composition, or object that can be used to cover, obstruct, fill, or seal any type of wound, ulcer, injury, hole, leak, cavity, enclosure, or opening in any tissue, organ or part of any organism as well as any composition, substance, or object that can have a hemostatic effect or can otherwise prevent, reduce, or eliminate the leakage, flow, or release of any substance (including liquid, solid, semisolid, and gas) into or out of the body of an organism or any part thereof. Sealants and tissue sealants can include, but are not limited to electroprocessed materials and matrices comprising electroprocessed materials.

The terms “electroprocessing” and “electrodeposition” shall be defined broadly to include all methods of electrospinning, electrospraying, electroaerosoling, and electrosputtering of materials, combinations of two or more such methods, and any other method wherein materials are streamed, sprayed, sputtered or dripped across an electric field and toward a target. The electroprocessed material can be electroprocessed from one or more grounded reservoirs in the direction of a charged substrate or from charged reservoirs toward a grounded target. “Electrospinning” means a process in which fibers are formed from a solution or melt by streaming a solution or melt through an orifice in response to an electric field. “Electroaerosoling” means a process in which droplets are formed from a solution or melt by streaming a polymer solution or melt through an orifice in response to an electric field. The term electroprocessing is not limited to the specific examples set forth herein, and it includes any means of using an electrical field for depositing a material on a target. The material may be in the form of fibers, powder, droplets, particles, or any other form. The target may be a solid, semisolid, liquid, or any other material.

The term “material” refers to any compound, molecule, substance, or group or combination thereof that is electroprocessed to from any type of structure or group of structures. Specifically, “material” refers to a compound, molecule, substance or combination thereof as it exists prior to electroprocessing. Materials include natural materials, synthetic materials, or combinations thereof. Naturally occurring organic materials include any substances naturally found in the body of animals, in plants or in other organisms, regardless of whether those materials have or can be synthetically produced or altered. Synthetic materials include any materials prepared through methods of artificial synthesis, processing, or manufacture. Preferably the materials are biologically compatible materials.

The term “electroprocessed material” refers to any composition that results from electroprocessing a “material” as defined herein, irrespective of the degree to which the resulting composition differs in chemical identity, physical structure or any other respect from the starting “material” that existed prior to electroprocessing. Further, similar terms that refer to the composition resulting from a specific type of electroprocessing (e.g. “electrospun material,” “electrosprayed material,” etc.) refer to any composition that results from performing that particular type of electroprocessing upon a “material” as defined herein, also irrespective of the degree to which the resulting composition differs in chemical identity, physical structure or any other respect from the starting “material” prior to electroprocessing. The foregoing definitions also apply where words such as “electroprocessed” are used to describe a specific compound, molecule, substance, or class or group thereof. Thus, for example, a reference to “electrospun fibrinogen” refers to the product of electrospinning fibrinogen, irrespective of whether that product actually constitutes or contains fibrinogen or any of the starting “materials” that were subjected to electroprocessing.

Proteins are a preferred class of materials for electroprocessing to make the tissue sealants of the present invention. Extracellular matrix proteins are a preferred class of proteins in the present invention. Examples of preferred proteins for electroprocessing include, but are not limited to, collagen, fibrin, fibrinogen, thrombin, elastin, laminin, and fibronectin. An especially preferred group of proteins for electroprocessing in the present invention is collagen, fibrinogen, fibrin, and thrombin of any type. Additional preferred materials for electroprocessing are other components of the extracellular matrix, for example proteoglycans. In each case, those names are used throughout the present application in their broadest definition and encompass the various isoforms that are commonly recognized to exist within the different families of proteins and other molecules. There are multiple types of each of these proteins and molecules that are naturally-occurring, as well as types that can be or are synthetically manufactured or produced by genetic engineering. For example, collagen occurs in many forms and types, and all of these forms and types are encompassed herein.

The term “protein,” and any term used to define a specific protein or class of proteins further includes, but is not limited to, protein fragments, protein analogs, and conservative amino acid substitutions, non-conservative amino acid substitutions and substitutions with non-naturally occurring amino acids with respect to a protein. Thus, for example, the term “collagen” includes, but is not limited to, fragments, analogs, conservative amino acid substitutions, and substitutions with non-naturally occurring amino acids or residues with respect to any type or class of collagen. The term “fibrinogen” includes, but is not limited to, fragments, analogs, conservative amino acid substitutions, and substitutions with non-naturally occurring amino acids or residues with respect to any type or class of fibrinogen. Thus, the term includes, for example the alpha chain of fibrinogen, the beta chain of fibrinogen, or a combination of both. As another example, the term “fibrin” includes, but is not limited to, fragments, analogs, conservative amino acid substitutions, and substitutions with non-naturally occurring amino acids or residues with respect to any type or class of fibrin. The term “residue” is used herein to refer to an amino acid (D or L) or an amino acid mimetic that is incorporated into a protein by an amide bond. As such, the residue can be a naturally occurring amino acid or, unless otherwise limited, can encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, one of skill in the art will recognize that, individual substitutions, deletions or additions which alter, add or delete a single amino acid or a small percentage of amino acids (preferably less than 10%, more preferably less than 5%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

It is to be understood that the term protein, polypeptide or peptide (as well as the reference to any specific type of proteins such as, for example, “collagen” or “fibrin”) further includes fragments that may be 90 to 95% of the entire amino acid sequence, and also extensions to the entire amino acid sequence that are 5% to 10% longer than the amino acid sequence of the protein, polypeptide or peptide.

When peptides are relatively short in length (i.e., less than about 50 amino acids), they are often synthesized using standard chemical peptide synthesis techniques. Techniques for solid phase synthesis are known to those skilled in the art. Alternatively, the proteins or peptides that may be electroprocessed are synthesized using recombinant nucleic acid methodology. Techniques sufficient to guide one of skill through such procedures are found in the literature.

When several desired protein fragments or peptides are encoded in the nucleotide sequence incorporated into a vector, one of skill in the art will appreciate that the protein fragments or peptides may be separated by a spacer molecule such as, for example, a peptide, consisting of one or more amino acids. Generally, the spacer will have no specific biological activity other than to join the desired protein fragments or peptides together, or to preserve some minimum distance or other spatial relationship between them. However, the constituent amino acids of the spacer may be selected to influence some property of the molecule such as the secondary structure, folding, net charge, or hydrophobicity. Nucleotide sequences encoding for the production of residues which may be useful in purification of the expressed recombinant protein may be built into the vector. Such sequences are known in the art. For example, a nucleotide sequence encoding for a poly histidine sequence may be added to a vector to facilitate purification of the expressed recombinant protein on a nickel column.

Once expressed, recombinant peptides, polypeptides and proteins can be purified according to standard procedures known to one of ordinary skill in the art, including ammonium sulfate precipitation, affinity columns, column chromatography, gel electrophoresis and the like. Substantially pure compositions of about 50 to 99% homogeneity are preferred, and 80 to 95% or greater homogeneity are most preferred for use as therapeutic agents.

Also, molecules capable of forming some of the named proteins can be mixed with other polymers during electroprocessing to obtain desired properties for uses of the formed protein in the matrix.

Another class of synthetic materials, preferably biologically compatible synthetic materials, comprises polymers. Such polymers include but are not limited to the following: poly(urethanes), poly(siloxanes) or silicones, polydioxanone, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, and polyorthoesters or any other similar synthetic polymers that may be developed that are biologically compatible. The term “biologically compatible, synthetic polymers” shall also include copolymers and blends, and any other combinations of the forgoing either together or with other polymers generally. The use of these polymers will depend on given applications and specifications required. A more detailed discussion of some polymers and types of polymers is set forth in Brannon-Peppas, Lisa, “Polymers in Controlled Drug Delivery,” Medical Plastics and Biomaterials, November 1997, which is incorporated by reference as if set forth fully herein.

“Materials” also include electroprocessed materials that are capable of changing into different materials during or after electroprocessing. For example, procollagen will form collagen when combined with procollagen peptidase. Procollagen, procollagen peptidase, and collagen are all within the definition of materials. Similarly, the protein fibrinogen, when combined with thrombin, forms fibrin. Other proteins and factors in the coagulation cascade serve in the formation of thrombin, fibrinogen, and fibrin, as well as the conversion of fibrin monomers into fibrin polymers. Any of these proteins and/or factors, and combinations of these proteins and/or factors that are electroprocessed as well as the fibrin that later forms are included within the definition of materials.

“Materials” also include any combination of materials. Combinations of natural materials, combinations of synthetic materials, and combinations of both natural and synthetic materials are included within the invention. Examples of combinations include, but are not limited to: blends of different types of collagen (e.g. Type I with Type II, Type I with Type III, Type II with Type III, etc.); blends of one or more types of collagen with fibrinogen, thrombin, elastin, PGA, PLA, PGA and PLA, polydioxanone; and blends of fibrinogen with one or more types of collagen, thrombin, elastin, PGA, PLA, PGA and PLA, or polydioxanone.

The sealants of the present invention contain electroprocessed materials. In a preferred embodiment, the electroprocessed materials in the sealants form a matrix. The term “matrix” refers to any structure comprised of electroprocessed materials. Matrices are comprised of fibers, particles, powders, or droplets of materials, or blends of fibers, particles, powders and droplets of any size or shape. Matrices are single structures or groups of structures and can be formed through one or more electroprocessing methods using one or more materials. Matrices are engineered to possess specific porosities. Substances can be deposited within, or anchored to or placed on matrices. Cells are substances which can be deposited within or on matrices.

The term “substance” shall be used throughout this application in its broadest definition. The term substance includes one or more molecules, objects, or cells of any type or size, or combinations thereof. Substances can be in any form including, but not limited to solid, semisolid, wet or dry mixture, gas, solution, suspension, and combinations thereof. Substances include molecules of any size and in any combination. Cells include all types of prokaryotic and eukaryotic cells, whether in natural state, or altered by genetic engineering or any other process. Cells can be from a natural source or cultured in vitro and can be living or dead. Combinations of different types of cells can be used. Objects can be of any size, shape, and composition that may be combined with or coupled to an electroprocessed material. Examples of objects include, but are not limited to, cell fragments, cell debris, fragments of cell walls, extracellular matrix constituents, fragments of viral walls, organelles and other cell components, tablets, viruses, vesicles, liposomes, capsules, nanoparticulates, and other structures that serve as an enclosure for molecules. The compositions of the present invention may comprise one substance or any combination of substances.

Throughout this application the term “solution” is used to describe liquids, such as liquids in the reservoirs of the electroprocessing process. The term is defined broadly to include any liquids. It is to be understood that any solutions capable of forming a material during electroprocessing are included within the scope of the present invention. In this application, the term “solution” also refers to suspensions or emulsions containing the material or anything to be electroprocessed. “Solutions” can be in organic or biologically compatible forms. This broad definition is appropriate in view of the large number of solvents or other liquids and carrier molecules, such as poly(ethylene oxide) (PEO), that can be used in the many variations of electroprocessing. In this application, the term “solution” also refers to melts, hydrated gels and suspensions containing the materials, substances or anything to be electroprocessed.

### Solvents

Any solvent can be used that allows delivery of the material or substance to the orifice, tip of a syringe, or other site from which the material will be electroprocessed in making the sealant. The solvent may be used for dissolving or suspending the material or the substance to be electroprocessed. Solvents useful for dissolving or suspending a material or a substance depend on the material or substance. Any solvents that do not unacceptably compromise the ability of the material to be electroprocessed or the desired properties of the material may be used. Electro spinning techniques often require specific solvent conditions. For example, collagen can be electroprocessed as a solution or suspension in water, 2,2,2-trifluoroethanol (also referred to herein as TFE), 1,1,1,3,3,3-hexafluoro-2-propanol (also referred to herein as hexafluoroisopropanol or HFP), isopropanol, or combinations thereof. Fibrin monomer can be electroprocessed from solvents such as urea, HFP and minimal essential medium (MEM) with Earle's balanced salts, monochloroacetic acid, water, 2,2,2-trifluoroethanol, HFP, or combinations thereof. Fibrinogen, as well as blends of fibrinogen and collagen, can be electroprocessed from, for example HFP, HFP and an aqueous solutions (for example, minimal essential medium (MEM) with Earle's balanced salts (without L-glutamine or sodium bicarbonate)), monochloroacetic acid, water, 2,2,2-trifluoroethanol, or combinations thereof. Elastin can be electroprocessed as a solution or suspension in water, 2,2,2-trifluoroethanol, isopropanol, HFP, or combinations thereof, such as isopropanol and water. In one desirable embodiment, elastin is electrospun from a solution of 70% isopropanol and 30% water containing 250 mg/ml of elastin. Other lower order alcohols, especially halogenated alcohols, may be used. Other solvents that may be used or combined with other solvents in electroprocessing natural matrix materials include acetamide, N-methylformamide, N,N-dimethylformamide (DMF), dimethylsulfoxide (DMSO), dimethylacetamide, N-methylpyrrolidone (NMP), acetic acid, trifluoroacetic acid, ethyl acetate, acetonitrile, trifluoroacetic anhydride, 1,1,1-trifluoroacetone, formic acid, maleic acid, hexafluoroacetone.

Some materials, including many proteins and peptides associated with membranes are hydrophobic and thus do not dissolve readily in aqueous solutions. Such proteins can be dissolved in organic solvents such as methanol, chloroform, and TFE and emulsifying agents. Any other solvents known to one of skill in the protein chemical art may be used, for example solvents useful in chromatography, especially high performance liquid chromatography. Proteins and peptides are also soluble, for example, in HFP, propanol, hexafluoroacetone, chloroalcohols in conjugation with aqueous solutions of mineral acids, dimethylacetamide containing 5% lithium chloride, and in acids such as acetic acid, hydrochloric acid and formic acid. In some embodiments, the acid solutions are dilute; in others, they are not. In some embodiments, concentrated formic acid is used. N-methyl morpholine-N-oxide is another solvent that can be used with many polypeptides. Other examples, used either alone or in combination with organic acids or salts, include the following: triethanolamine; dichloromethane; methylene chloride; 1,4-dioxane; acetonitrile; ethylene glycol; diethylene glycol; ethyl acetate; glycerine; propane-1,3-diol; furan; tetrahydrofuran; indole; piperazine; pyrrole; pyrrolidone; 2-pyrrolidone; pyridine; quinoline; tetrahydroquinoline; pyrazole; and imidazole. Combinations of solvents may also be used.

Synthetic polymers may be electroprocessed from, for example, HFP, methylene chloride, ethyl acetate; acetone, 2-butanone (methyl ethyl ketone), diethyl ether; ethanol; cyclohexane; water; dichloromethane (methylene chloride); tetrahydrofuran; dimethylsulfoxide (DMSO); acetonitrile; methyl formate and various solvent mixtures. HFP and methylene chloride are desirable solvents. Selection of a solvent will depend upon the characteristics of the synthetic polymer to be electroprocessed.

Selection of a solvent is based in part on consideration of secondary forces that stabilize polymer-polymer interactions and the solvent's ability to replace these with strong polymer-solvent interactions. In the case of polypeptides such as collagen, and in the absence of covalent crosslinking, the principal secondary forces between chains are: (1) coulombic, resulting from attraction of fixed charges on the backbone and dictated by the primary structure (e.g., lysine and arginine residues will be positively charged at physiological pH, while aspartic or glutamic acid residues will be negatively charged); (2) dipole-dipole, resulting from interactions of permanent dipoles; the hydrogen bond, commonly found in polypeptides, is the strongest of such interactions; and (3) hydrophobic interactions, resulting from association of non-polar regions of the polypeptide due to a low tendency of non-polar species to interact favorably with polar water molecules. The stabilization of polypeptide secondary structures in solvents is believed desirable, especially in the cases of collagen and elastin, to preserve the proper formation of collagen fibrils during electroprocessing.

Additionally, it is often desirable, although not necessary, for the solvent to have a relatively high vapor pressure to promote the stabilization of an electrospinning jet to create a fiber as the solvent evaporates. A relatively volatile solvent is also desirable for electrospraying to minimize coalescence of droplets during and after spraying and formation of dry electroprocessed materials. In embodiments involving higher boiling point solvents, it is often desirable to facilitate solvent evaporation by warming the spinning or spraying solution, and optionally the electroprocessing stream itself, or by electroprocessing in reduced atmospheric pressure or elevated ambient temperature. It is also believed that creation of a stable jet resulting in a fiber is facilitated by a low surface tension of the polymer/solvent mixture. Solvent choice can also be guided by this consideration.

In functional terms, solvents used for electroprocessing have the principal role of creating a mixture with materials to be electroprocessed such that electroprocessing is feasible. The concentration of a given solvent is often an important consideration in determining the type of electroprocessing that will occur. For example, in electrospraying, the solvent should assist in the dispersion of droplets of electroprocessed material so that the initial jet of liquid disintegrates into droplets. Accordingly, solvents used in electrospraying should not create forces that will stabilize an unconfined liquid column. In electrospinning, interactions between molecules of electroprocessed material stabilize the jet, leading to fiber formation. For electrospun embodiments, the solvent should sufficiently dissolve or disperse the polymer to prevent the jet from disintegrating into droplets and should thereby allow formation of a stable jet in the form of a fiber. In some embodiments, the transition from electrospraying to electrospinning can be determined by examining viscosity measurements (using a Brookfield viscometer) for polymer solutions as a function of concentration. Viscosity increases as concentration of a polymer or other material to be electroprocessed increases. Above a critical concentration associated with extensive chain entanglements of materials, however, the viscosity will increase more rapidly with concentration, as opposed to a more gradual, linear rise with concentration at lower concentrations. Departures from linearity approximately coincide with the transition from electrospraying to electrospinning.

The solubility of any electroprocessed material in a solvent may be enhanced by modifying the material. Any method for modifying materials to increase their solubility may be used. For example, U.S. Pat. No. 4,164,559 to Miyata et al. discloses a method for chemically modifying collagen to increase solubility.

In some embodiments, a solvent is selected based on its compatibility with one or more substances in the electroprocessed material. For example, in some embodiments, nerve growth factor retains a higher degree of biological activity when electroprocessed from TFE than when electroprocessed from HFP.

In some embodiments solvents are selected based on their effect upon variance in fiber diameter in a resulting electrospun composition and the degree to which such variance increases with concentration. For example, in some embodiments, electrospinning Type I collagen from TFE results in greater variation in fiber diameter than electrospinning the same collagen from HFP.

In some embodiments, solvents are selected based on their effect upon dimensions such as pore size in the resulting electroprocessed composition. For example, in some embodiments, electrospinning Type I collagen from TFE results in a matrix having a greater pore dimension (a term referring to the average distance between fibers in one plane) than electrospinning the same collagen from HFP.

### Tissue Sealant Compositions of the Present Invention

### The Electroprocessed Material

One component of the tissue sealants of the present invention is the electroprocessed material. As defined above, the electroprocessed material of the present invention can result from the electroprocessing of natural materials, synthetic materials, or combinations thereof. Examples include but are not limited to amino acids, peptides, denatured peptides such as gelatin from denatured collagen, polypeptides, proteins, carbohydrates, lipids, nucleic acids, glycoproteins, lipoproteins, glycolipids, glycosaminoglycans, and proteoglycans.

Some preferred materials to be electroprocessed are naturally occurring extracellular matrix materials and blends of naturally occurring extracellular matrix materials, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, and proteoglycans. Especially preferred materials for electroprocessing include collagen, fibrin, fibrinogen, thrombin, fibronectin, and combinations thereof. Some collagens that are used include but are not limited to collagen types I, II, III, IV, V, VI, VII, VIII, IX, X, XI, XII, XIII, XIV, XV, XVI, XVII, XVIII, and XIX. Some preferred collagens include types I, II, and III. These proteins may be in any form, including but not limited to native and denatured forms. Other preferred materials for electroprocessing are carbohydrates such as polysaccharides (e.g. cellulose and its derivatives), chitin, chitosan, alginic acids, and alginates such as calcium alginate and sodium alginate. These materials may be isolated from humans or other organisms or cells or synthetically manufactured. Some especially preferred natural materials for electroprocessing are collagen, fibrinogen, thrombin, fibrin, fibronectin, and combinations thereof. Also included are crude extracts of tissue, extracellular matrix material, extracts of non-natural tissue, or extracellular matrix materials (i.e. extracts of cancerous tissue), alone or in combination. Extracts of biological materials, including but are not limited to cells, tissues, organs, and tumors may also be electroprocessed.

Collagen and fibrinogen have each been electrospun to produce fibers having repeating, band patterns along the length of the fibers. These patterns are observable, for example with transmission electron microscopy, and are typical of those produced by natural processes. In some embodiments, the banded pattern observed in electrospun collagen fibers is the same as that produced by cells in vivo. In some embodiments, the banding pattern in electrospun fibrinogen is the same as that of fibrinogen found in normal clots formed in vivo. While not wanting to be bound by the following statement, it is believed that the banding apparent along natural collagen fibers results from the helical pattern of the protein chains in the collagen, while the banding in fibrinogen in vivo results from close packing of individual fibrin molecules in a stacked configuration. In the latter case, it is further believed that, in the case of fibrinogen, the banding patterns in normal clots may be due to physical entrapment and juxtaposition of individual, unpolymerized fibrinogen molecules by the fibrin structure of the clot rather than polymerization of the fibrinogen. However, in some embodiments electroprocessed fibrinogen fibers have this banding pattern without being entrapped within a clot. In some of these embodiments, the compositions that are composed of fibrous webs rather than networks characteristic of fibrin clots. Further, in some embodiments, electroprocessed fibrinogen is not soluble in water, unlike native fibrinogen.

In some embodiments (including some embodiments including type I, II, and III collagen), collagen is electrospun such that it has a banding pattern repeats about every 65-70 nm along the fiber. In some embodiments, the banding pattern repeats about 65 nm. In other embodiments, the banding pattern is about 67 nm. In a preferred embodiment, the banded pattern characteristic of electrospun collagen is an important attribute because it allows cells to have access to active sites within the collagen molecule that promote or regulate specific activities. In other embodiments, including some embodiments involving electrospun denatured collagen from gelatin, the characteristic banding patterns are absent. In some embodiments, electrospun fibrinogen has a banding pattern of approximately 20-25 nm. In other embodiments the banding pattern is about 22.5 nm. As can be seen from the examples disclosed, herein, blends or combinations of different materials are used in some embodiments. Such blends or combinations are used to duplicate one or more naturally occurring blends or combinations, or to prepare a composition that is entirely unique and differ from any natural blend or combination.

When tissue sealants are electroprocessed from natural materials (e.g. proteins, peptides, nucleic acids, glycosaminoglycans and proteoglycans) for implantation or other administration to an organism, those materials can include, but are not limited to, autologous materials, materials from a conspecific organism, or materials from another species. Material from any species or combination of species can be used. Natural molecules that are produced synthetically can include those produced by any artificial means. Numerous methods for producing fibrins, fibrinogen, thrombin, fibronectin, collagens and other proteins are known in the art. Synthetic proteins can be prepared using specific sequences. Proteins may be produced by any means, including, for example, peptide, polypeptide, or protein synthesis. Genetically engineered proteins can be prepared with specific desired sequences of amino acids that differ from natural proteins. For example, cells can be genetically engineered in vivo or in vitro to produce desired proteins or molecules capable of forming those proteins, or subdomains of desired proteins, and the proteins can be harvested. In one illustrative embodiment, desirable sequences that form binding sites on proteins (e.g. collagens) for cells or peptides can be included in higher amounts than found naturally in those proteins. The electroprocessed material may also be formed from proteins or any other material that forms the proteins while electroprocessing. Examples include, but are not limited, to amino acids, peptides, denatured proteins, polypeptides, and proteins. Proteins can be formed before, during, or after electroprocessing. For example, electroprocessed collagen formed by combining procollagen with procollagen peptidase before, during, or after electroprocessing is within the invention. Fibrin formed by polymerization of fibrinogen (either by the action of thrombin or by any other means) before, during, or after electroprocessing is also within the invention.

The invention includes all natural or synthetic compositions that result from the electroprocessing of any material. Materials that change in composition or structure before, during, or after electroprocessing are within the scope of the invention.

It is to be understood that these electroprocessed materials may be combined with other materials and/or substances in forming the compositions of the present invention. For example, in some embodiments an electroprocessed peptide is combined with an adjuvant to enhance immunogenicity when implanted subcutaneously. Electroprocessed materials in some embodiments are prepared at very basic or acidic pHs (for example, by electroprocessing from a solution having a specific pH) to accomplish the same effect. As another example, an electroprocessed matrix, containing cells, may be combined with an electroprocessed biologically compatible polymer and growth factors to stimulate growth and division of the cells in the electroprocessed matrix.

Synthetic materials electroprocessed for use in the sealants include any materials prepared through any method of artificial synthesis, processing, isolation, or manufacture. The synthetic materials are preferably biologically compatible for administration in vivo or in vitro. Such polymers include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Some preferred synthetic materials include PLA, PGA, copolymers of PLA and PGA, polycaprolactone, poly(ethylene-co-vinyl acetate), EVOH, PVA, and PEO. Polymers with cationic moieties are also preferred in some embodiments. Examples of such polymers include, but are not limited to, poly(allyl amine), poly(ethylene imine), poly(lysine), and poly(arginine). The polymers may have any molecular structure including, but not limited to, linear, branched, graft, block, star, comb and dendrimer structures. Matrices can be formed of electrospun fibers, electroaerosol, electrosprayed, or electrosputtered droplets, electroprocessed powders or particles, or a combination of the foregoing.

In embodiments of the sealants prepared by electroprocessing natural materials, those materials can be derived from a natural source, synthetically manufactured, or manufactured by genetically engineered cells. For example, in some embodiments genetically engineered proteins are prepared with specific desired sequences of amino acids that differ from the natural proteins. In one illustrative embodiment, desirable sequences that form binding sites for cells or peptides on a collagen, fibrin, or fibrinogen protein are included in higher amounts than found in natural proteins. For example, natural fibrinogen may be purified from plasma or prepared as a cryoprecipitate.

By selecting different materials, or combinations thereof, many characteristics of the tissue sealants are manipulated. The properties of the matrix comprised of electroprocessed material and a substance may be adjusted. As discussed in greater detail below, electroprocessed materials themselves can provide a therapeutic effect when applied. In addition, selection of materials for electroprocessing can affect the permanency of an implanted matrix. For example, many matrices made by electroprocessing fibrinogen or fibrin will degrade more rapidly while many matrices made of collagen are more durable and many other matrices made by electroprocessing materials are more durable still. Thus, for example, incorporation of durable synthetic polymers (e.g. PLA, PGA) will increase the durability and structural strength of matrices electroprocessed from solutions of fibrinogen in some embodiments. Use of matrices made by electroprocessing natural materials such as proteins also minimize rejection or immunological response to an implanted matrix. Accordingly, selection of materials for electroprocessing and use in substance delivery is influenced by the desired use. In one embodiment, a skin patch of material electroprocessed from fibrin, fibrinogen, fibronectin, collagen or a combination of one or more of these is combined with healing promoters, analgesics and or anesthetics and anti-rejection substances and applied to the skin and may subsequently dissolve into the skin. In another embodiment, an electroprocessed implant for delivery to bone may be constructed of materials useful for promoting bone growth, osteoblasts and hydroxyapatite, and may be designed to endure for a prolonged period of time. In embodiments in which the matrix contains substances that are to be released from the matrix, incorporating electroprocessed synthetic components, such as biocompatible substances, can modulate the release of substances from an electroprocessed composition. For example, layered or laminate structures can be used to control the substance release profile. Unlayered structures can also be used, in which case the release is controlled by the relative stability of each component of the construct. For example, layered structures composed of alternating electroprocessed materials are prepared by sequentially electroprocessing different materials onto a target. The outer layers are, for example, tailored to dissolve faster or slower than the inner layers. Multiple agents can be delivered by this method, optionally at different release rates. Layers can be tailored to provide a complex, multi-kinetic release profile of a single agent over time. Using combinations of the foregoing provides for release of multiple substances released, each with its own profile. Complex profiles are possible.

Synthetic components such as biocompatible substances can be used to modulate the release of electroprocessed materials or of substances from an electroprocessed sealant composition. For example, a drug or series of drugs or other materials or substances to be released in a controlled fashion can be electroprocessed into a series of layers. In one embodiment, one layer is composed of electroprocessed fibrinogen plus a drug, the next layer PLA plus a drug, a third layer is composed of polycaprolactone plus a drug. The layered construct can be implanted, and as the successive layers dissolve or break down, the drug (or drugs) is released in turn as each successive layer erodes. In some embodiments, unlayered structures are used, and release is controlled by the relative stability of each component of the construct. Another advantage of the synthetic materials is that different solvents can be used. This can be important for the delivery of some materials. For example, a drug may be soluble in some organics, and using synthetics increases the number of materials that can be electroprocessed. The breakdown of these synthetic materials can be tailored and regulated in ways that are not available to natural materials. The synthetics are usually not subject to enzymatic breakdown, and many spontaneously undergo hydrolysis. In addition to these characteristics, substances can be released from electroprocessed materials in response to electrical, magnetic and light based signals. Polymers that are sensitive to such signals can be used, or the polymers may be derivatized in a way to provide such sensitivity. These properties provide flexibility in making and using electroprocessed materials designed to deliver various substances, in vivo and in vitro.

In some embodiments of the sealants of the present invention, the electroprocessed material itself may act as a sealant and may provide a therapeutic effect. One embodiment of that have a therapeutic effect is electroprocessed fibrinogen, thrombin, fibrin, or combinations thereof. Thrombin converts fibrinogen to fibrin. Fibrin assists in arrest of bleeding (hemostasis). Fibrin is a component of the provisional matrix that is laid down during the early stages of healing and may also promote the growth of vasculature in adjacent region. In many ways fibrin is a natural healing promoter. In some embodiments, electroprocessed fibrinogen also assists in healing. When placed in contact with a wound of a patient, such an electroprocessed material provides the same healing properties as fibrin.

As another example, in some embodiments electroprocessed collagen promotes cellular infiltration and differentiation, so a sealant containing electroprocessed collagen matrix assists with healing. The P-15 site, a 15 amino acid sequence within the collagen molecule, promotes osteoblasts to produce and to secrete hydroxyapatite, a component of bone. Another example of specific sites and sequences within collagen molecules that can be manipulated and processed in a similar fashion includes the RGD binding sites of the integrin molecule. The RGD site is a sequence of three amino acids (Arg-Gly-Asp) present in many extracellular matrix molecules that serves as a binding site for cell adhesion. It is recognized and bound, for example, by integrins. In addition, electroprocessed materials can be enriched with specific desired sequences before, during, or after electroprocessing. This can be done by any means including, but not limited to, use of recombinant nucleic acids. Sequences can be added in linear or other forms. In some embodiments, the RGD sequences are arranged in a cyclic form referred to as cycloRGD.

An electroprocessed sealant, such as a sealant in the form of a matrix, can also be composed of specific subdomains of a matrix constituent and can be prepared with a synthetic backbone that can be derivatized. For example, the RGD peptide sequence, and/or a heparin binding domain and/or other sequences, can be chemically coupled to synthetic materials. The synthetic polymer with the attached sequence or sequences can be electroprocessed into a construct. This produces a matrix with unique properties. In these examples the RGD site provides a site for cells to bind to and interact with the matrix. The heparin-binding site provides a site for the anchorage of peptide growth factors to the synthetic backbone. Angiogenic peptides, genetic material, growth factors, cytokines, enzymes and drugs are other non-limiting examples of substances that can be attached to the backbone of an electroprocessed material to provide functionality. Peptide side chains may also be used to attach molecules to functional groups on polymeric backbones. Molecules and other substances can be attached to a material to be electroprocessed by any technique known in the art.

Electroprocessed material may also be made of a molecular structure that is tailored to increase surface area to volume ratio of the electroprocessed material and thereby enhance hemostatic or other desired properties. In some embodiments, substances or moieties that enhance these functions (for example, thrombin) are attached to the electroprocessed material and thereby increase the performance of the sealant. In some embodiments, the number of alpha 2 beta 1 binding sites (e.g. the GFOGER sequence) are increased on the sealant through the use of engineered peptides (e.g. prepared recombinantly), to promote platelet adhesion and the activation of the clotting cascade. Embodiments exist that use any type of engineered protein or peptide having sequences and structures manipulated through recombinant or other means.

The electroprocessed material in the sealants may be made using any electroprocessing technique, including, but not limited to, electrospinning, electroaerosol, electrospraying or electrosputtering techniques, or any combination thereof. Accordingly, electroprocessed droplets, particles, fibers, fibrils, or combinations thereof are all included in the electroprocessed compositions of the present invention. In one embodiment, the materials are electrospun to form fibers.

Synthetic electroprocessed materials are made using any materials prepared through any method of artificial synthesis, processing, or manufacture. The synthetic materials are preferably biologically compatible for administration in vivo or in vitro.

Layering of structures is used in some sealants in which it is desired to mimic more closely the composition of natural materials. For example, providing a sealant with selected amounts of Type I collagen, Type III collagen, and elastin in successive layers is used in some embodiments to mimic gradients or other patterns of distribution across the depth of a structure such as the wall of a blood vessel. Other embodiments accomplish such patterns without layering. For example, altering the feed rates of Type I collagen, Type III collagen and elastin into an electroprocessing apparatus during an electroprocessing run allows for creation of continuous gradients in sealant compositions and patterns in sealant compositions without layering. In some embodiments, amounts of electroprocessed collagen, fibrinogen, thrombin, and/or fibronectin are varied throughout a composition by layering or patterned application.

Synthetic materials can be electroprocessed from different solvents. This can be important for uses of sealants in the delivery of some materials. In some embodiments, a drug that is insoluble in the solvents used to electroprocess proteins will be soluble in a solvent used to electroprocess synthetic materials. In such embodiments, using synthetics increases the number of chemicals that can be combined with the electroprocessed matrix in the sealant. Polymers can be derivatized in a way to provide this feature. These properties provide flexibility in making and using electroprocessed materials designed to deliver various substances, in vivo and in vitro.

Substances Combined with Electroprocessed Materials in the Sealants

In many desirable embodiments, the electroprocessed materials in the sealants are combined with one or more substances. As discussed above, the word “substance” in the present invention is used in its broadest definition. In embodiments in which the electroprocessed compositions of the present invention comprise one or more substances, substances can include any type or size of molecules, cells, objects or combinations thereof. The compositions of the present invention may comprise one substance or any combination of substances.

Some embodiments of the sealants include cells as a substance combined with the electroprocessed matrix. Any cell can be used. Some preferred examples include, but are not limited to, stem cells, committed stem cells, and differentiated cells. Examples of stem cells include, but are not limited to, embryonic stem cells, bone marrow stem cells, muscle derived stem cells, and umbilical cord stem cells. Other examples of cells used in various embodiments include, but are not limited to, osteoblasts, myoblasts, neuroblasts, fibroblasts, glioblasts, germ cells, hepatocytes, chondrocytes, chondroblasts, osteocytes, keratinocytes, smooth muscle cells, cardiac muscle cells, connective tissue cells, glial cells, epithelial cells, endothelial cells, hormone-secreting cells, cells of the immune system, and neurons. In some embodiments it is unnecessary to pre-select the type of stem cell that is to be used, because many types of stem cells can be induced to differentiate in an organ specific pattern once delivered to a given organ or implant site. For example, a stem cell delivered to the liver can be induced to become a liver cell simply by placing the stem cell within the liver. Cells in the matrix can serve the purpose of providing scaffolding or seeding, producing certain compounds, or both.

Embodiments in which the substance comprises cells include cells that can be cultured in vitro, derived from a natural source, genetically engineered, or produced by any other means. Any natural source of prokaryotic or eukaryotic cells may be used. Synthetic sources such as transgenic organisms or cells that have been engineered through such techniques as nuclear transplantation, can also be used as a source of cells. Embodiments in which the matrix is implanted in an organism can use cells from the recipient, cells from a conspecific donor or a donor from a different species, or bacteria or microbial cells. Cells harvested from a source and cultured prior to use are included. Cells may be living or dead.

Some embodiments use cells that are abnormal in some way. Examples include cells that have been genetically engineered, transformed cells, and immortalized cells. Genetic engineering includes programming the cell to express one or more genes, repressing the expression of one or more genes, or both. One example of genetically engineered cells useful in the present invention is a genetically engineered cell that makes and secretes one or more desired molecules. When genetically engineered cells are implanted in an organism, the molecules produced can produce a local effect or a systemic effect, and can include the molecules identified above as possible substances. Cells can also produce antigenic molecules in embodiments in which one of the purposes of the matrix is to produce an immune response. Cells may produce substances to aid in the following non-inclusive list of purposes: promote hemostasis; seal or close an opening or form a bond between a tissue or organ and another object; provide reinforcement to a structure or connection; inhibit or stimulate inflammation; facilitate healing; resist immunorejection; provide hormone replacement; replace neurotransmitters; inhibit or destroy cancer cells; serve as a filler and sealant for sites where tissue, organs or tumors have been removed; promote cell growth; inhibit or stimulate formation of blood vessels; augment tissue; and to supplement or replace neurons, skin, synovial fluid, tendons, cartilage, ligaments, bone, muscle, organs, dura, blood vessels, bone marrow, and extracellular matrix. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

In many embodiments, cells in an electroprocessed matrix exhibit characteristics and functions typical of such cells in vivo. Examples include, but are not limited to: chondrocytes in a Type II collagen matrix causing cell adhesion and formation in the matrix of lacunae of the type characteristic of cartilage in vivo; immortalized chondrocytes in an electroprocessed Type II collagen matrix forming cell clusters characteristic of immortalized chondrocytes in vivo; immortalized chondrocytes in an electroprocessed fibrinogen matrix forming cell clusters characteristic of immortalized chondrocytes in vivo; immortalized chondrocytes in a Type I collagen matrix forming cell clusters characteristic of immortalized chondrocytes in vivo; and osteoblasts in a Type I collagen matrix that differentiate and produce hydroxyapatite. Embodiments in which cells exhibit either normal, abnormal, or a combination of normal and abnormal characteristics are within the present invention.

In embodiments in which the substances are molecules, any molecule can be used.

Molecules may, for example, be organic or inorganic and may be in a solid, semisolid, liquid, or gas phase. Molecules may be present in combinations or mixtures with other molecules, and may be in solution, suspension, or any other form. Examples of classes of molecules that may be used include human or veterinary therapeutics, cosmetics, nutraceuticals, agriculturals such as herbicides, pesticides and fertilizers, vitamins, salts, electrolytes, amino acids, peptides, polypeptides, proteins, carbohydrates, lipids, nucleic acids, glycoproteins, lipoproteins, glycolipids, glycosaminoglycans, proteoglycans, growth factors, hormones, neurotransmitters, pheromones, chalones, prostaglandins, immunoglobulins, monokines and other cytokines, humectants, metals, gases, minerals, plasticizers, ions, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Preferred molecules include hemostatic molecules, other molecules that facilitate clotting, anti-immunorejection molecules, extracellular matrix molecules, and molecules that inhibit fibrinolysis.

Several preferred embodiments include use of any therapeutic molecule including, without limitation, any pharmaceutical or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfuram and disulfuram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All substances listed by the U.S. Pharmacopeia are also included within the substances of the present invention.

Other preferred embodiments involve the use of growth factors. Growth factors useful in the present invention include, but are not limited to, transforming growth factor-a (“TGF-α”), transforming growth factor-β (“TGF-β”), platelet-derived growth factors including the AA, AB and BB isoforms (“PDGF”), fibroblast growth factors (“FGF”), including FGF acidic isoforms 1 and 2, FGF basic form 2, and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), epithelial growth factor (EGF), keratinocyte growth factor (KGF), transforming growth factors (TGF), including TGFs alpha, beta, beta1, beta2, and beta3, skeletal growth factor, bone matrix derived growth factors, and bone derived growth factors and mixtures thereof.

Cytokines useful in the present invention include, but are not limited to, cardiotrophin, stromal cell derived factor, macrophage derived chemokine (MDC), melanoma growth stimulatory activity (MGSA), macrophage inflammatory proteins 1 alpha (MIP-1alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, and TNF-β. Immunoglobulins useful in the present invention include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as substances in the present invention include, but are not limited to, growth hormones, leptin, leukemia inhibitory factor (LIF), tumor necrosis factor alpha and beta, endostatin, angiostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5, 6, 7, 8, 9, 10, 11, 12, 13, 15, 16, 17 and 18.

Embodiments involving amino acids, peptides, polypeptides, and proteins may include any type of such molecules of any size and complexity as well as combinations of such molecules. Examples include, but are not limited to, structural proteins, enzymes, and peptide hormones. These compounds can serve a variety of functions. In some embodiments, the matrix may contain peptides containing a sequence that suppresses enzyme activity through competition for the active site. In other applications antigenic agents that promote an immune response and invoke immunity can be incorporated into a construct.

For substances such as nucleic acids, any nucleic acid can be present. Examples include, but are not limited to deoxyribonucleic acid (DNA), ent-DNA, and ribonucleic acid (RNA). Embodiments involving DNA include, but are not limited to, cDNA sequences, natural DNA sequences from any source, and sense or anti-sense oligonucleotides. For example, DNA can be naked (e.g., U.S. Pat. Nos. 5,580,859; 5,910,488) or complexed or encapsulated (e.g., U.S. Pat. Nos. 5,908,777; 5,787,567). DNA can be present in vectors of any kind, for example in a viral or plasmid vector. In some embodiments, nucleic acids used will serve to promote or to inhibit the expression of genes in cells inside and/or outside the electroprocessed matrix. The nucleic acids can be in any form that is effective to enhance uptake into cells.

Substances in the electroprocessed sealant compositions of the present invention also comprise objects. Examples of objects include, but are not limited to, cell fragments, cell wall fragments, cellular fractions, cell debris, organelles and other cell components, tablets, and viruses as well as vesicles, liposomes, capsules, nanoparticles, and other structures that serve as an enclosure for molecules. In some embodiments, the objects constitute vesicles, liposomes, capsules, or other enclosures that contain compounds that are released at a time after electroprocessing, such as at the time of implantation or upon later stimulation or interaction. In one illustrative embodiment, transfection agents such as liposomes contain desired nucleotide sequences to be incorporated into cells that are located in or on the electroprocessed material or matrix. In other embodiments, cell fragments, specific cell fractions or cell debris are incorporated into the matrix. The presence of cell fragments is known to promote healing in some tissues.

Magnetically or electrically reactive materials are also examples of substances that are optionally included within the electroprocessed sealant compositions of the present invention. Examples of magnetically active materials include but are not limited to ferrofluids (colloidal suspensions of magnetic particles), and various dispersions of electrically conducting polymers. Ferrofluids containing particles approximately 10 nm in diameter, polymer-encapsulated magnetic particles about 1-2 μm in diameter, and polymers with a glass transition temperature below room temperature are particularly useful. Examples of electrically active materials are polymers including, but not limited to, electrically conducting polymers such as polyanilines and polypyrroles, ionically conducting polymers such as sulfonated polyacrylamides are related materials, and electrical conductors such as carbon black, graphite, carbon nanotubes, metal particles, and metal-coated plastic or ceramic materials.

In some embodiments, some substances in the tissue sealants supplement or augment the function of other substances. For example, when the composition comprises cells that express a specific gene, the composition can contain oligonucleotides that are taken up by the cells and affect gene expression in the cells. One or more agents that promote specific and non-specific uptake (for example, fibronectin) is optionally incorporated into the matrix to increase cellular uptake of oligonucleotides by pinocytosis.

The tissue sealants of the present invention can contain any electroprocessed materials and any substances or combinations of substances as discussed above. In a preferred embodiment, the tissue sealant containing electroprocessed collagen, fibrinogen, fibronectin, thrombin, synthetic polymers, or combinations thereof also contains other substances to assist coagulation or to provide other benefits. Any of the foregoing materials can be present as electrospun fibers or parts thereof, material electroprocessed by other means, or substances added by a means other than electroprocessing. Other preferred substances include coagulation factors and other factors and compounds involved in the coagulation cascade. For example, coagulation factors (e.g. factors I, II, III, IV, V, VI, VII, VIII, IX, X, XI, XII, and XIII, or combinations thereof) are included in some embodiments. Preferred substances also include coagulation factors present in their activated form (i.e. factors Ia, IIa, IIIa, IVa, Va, VIIa, VIIa, VIIIa, IXa, Xa, XIa, XIIa, and XIIIa or combinations thereof). Other preferred substances include other factors in the coagulation cascade or chemicals that inhibit fibrinolysis or otherwise inhibit breaking down of a clot. Examples include, but are no limited to, calcium ions (for example, CaCl₂), Von Willebrand factor, aprotinin, thrombin, prothrombin, thrombin mimetics, fibrinolysis inhibitors (including but not limited to thrombin-activated fibrinolytic inhibitor), 6-aminocaproic acid or epsilon-aminocaproic acid, and tranexamic acid ((4-aminomethyl)cyclohexanecarboxylic acid)). Fibronectin, plasma components, and platelet extracts and contents are also preferred matrix components in some embodiments of tissue sealants. In some embodiments, substances that promote fibrinolysis (e.g. tissue plasminogen activator (TPA), urokinase, streptokinase) and/or substances that inhibit clotting (e.g. heparin, coumarin) are included to slow coagulation or to cause the clot to dissipate after the passage of time. In some embodiments, the composition of the sealant is tailored to a patient with hemorrhagic disorder (e.g. von Willebrand's diseases, thrombasthenia hemophilia A or B, idiopathic thrombocytopenic purpura, deficiencies of factor VII or XI) by incorporating the deficient factor, mimetics for the deficient factor, or precursors for either. Embodiments exist that contain any natural, mimetic, or synthetic substance that will promote or cause coagulation, or combinations thereof. One example of natural materials that promote coagulation is snake venoms. Many snake venoms have a procoagulant effect. Examples include but are not limited to thrombocytin (from Bothrops atrox), certain molecules in the venom of Russell's Viper (including but not limited to RVV-V, RVV-X, and RVV-IX), Ecarin (from the Saw Sealed Viper), Tiger Snake activator (from the Tiger Snake), and Taipan venom (from the Taipan viper). Some venoms can promote fibronogen clotting, and thus serve as a thrombin mimetic. Examples of this type of venom include, but are not limited to Ancrod (from the Malayan Pit Viper), Batroxobin (from Bothrops atrox), Crotalase (from the Eastern Diamondback), Venzyne (from the Southern Copperhead), and Gabonase (from the Gabon Viper).

In some embodiments, the sealant includes a heparin antagonist (for example, protamine sulfate or Platelet Factor IV) in an amount and form effective to inactivate heparin. Such a sealant can, for example, minimize the local effect of heparinization in a patient, allowing heparinization systemically while locally treating a site where hemostasis is desired.

In some embodiments, the sealant includes a substance that is capable of forming bonds with natural tissues. Albumins and crosslinking agents such as glutaraldehyde and other aldehydes are examples.

In some embodiments, the sealant includes a substance that affects the degree or rate of dissolution or degradation of the sealant. For example, Type I collagen sealant and BONE SOURCE hydroxyapatite cement powder, (available from Stryker Leibinger GmbH & Co. KG, Freiburg, Germany,) a material that includes hydroxyapatite crystals and calcium chloride, were electroprocessed together from TFE. The TFE solution contained collagen at a concentration of 80 mg/ml and BONE SOURCE at a concentration of about 40-60 mg/ml (48 mg/ml in one embodiment). The resulting matrix was stable in water for at least 48 hours, even without crosslinking. Fibers swelled and coalesced somewhat due to hydration, but did not dissolve. Ordinarily, uncrosslinked electrospun collagen dissolves in water within minutes.

Hydration of a matrix in some embodiments results in formation of a hardened, porous structure. In one embodiment, hydrating a Type I collagen matrix containing the BONE SOURCE product described above resulted in a matrix that, upon hydration, became hardened and porous such that it possessed a structure similar to that of bone.

Exposing an electrospun collagen sealant to a surgical glue such as vapors of a cyanoacrylate retards hydration and swelling. This can be practiced with any electroprocessed material and any glue that is effective for this purpose. Any cyanoacrylate can be used as a glue. Examples include, but are not limited to, methyl cyanoacrylates, ethyl cyanoacrylates, butyl cyanoacrylates, octyl cyanoacrylates, allyl cyanoacrylates and methoxyethyl cyanoacrylates. Other substances useful in the same way as cyanoacrylates as a glue include, but are not limited to, isocyanates, metal alkoxides, and epoxides such as alkylene oxides. In some embodiments, the glues are substances that polymerize upon contact with water. In embodiments involving glues that are toxic prior to polymerization, polymerization can occur prior to application or implantation by placing the electroprocessed material in contact with water.

In some embodiments, electroprocessed materials or substances that absorb or otherwise entrap water are included in the electroprocessed sealant. One result is rendering a sealant less adhesive and more lubricious, which permits the sealant to move with respect to the underlying tissue. Such embodiments are useful in uses in which such movement is desired, (for example a membrane such as the native pericardium).

### Uses of the Electroprocessed Tissue Sealants

The electroprocessed tissue sealants of the present invention have many uses and are also within the present invention. The sealants are suitable to a wide variety of uses including but not limited to hemostatic agents, structural connections, scaffolds and supports, and obstruction or closure of leakages and other openings and cavities. One use is as a hemostatic agent to stop bleeding at the site of a wound, injury, or other bleed. The sealants are used both internally (e.g. upon blood vessels, gut linings, and organs) and externally (e.g. on the skin). Examples of external use include upon burns, especially after excision of burned tissue, abrasions, ulcers, cuts and punctures on any part of the body. In these embodiments the sealants serve, for example, as the sole component of a hemostatic bandage, as a component of a bandage that includes other elements such as adhesive backings, backings to provide a water barrier around the outside of the wound or site of application, and other substances (e.g. cytokines, growth factors, antibiotics, and medications). Sealants can serve as a temporary sealant until placement of a graft, or as an intermediate layer between a graft and the underlying wound. Examples of preferred substances included in such hemostatic agents and bandages include but are not limited to, agents that slow blood delivery, for example by producing arterial constriction, keratinocyte growth factors, antibiotics, and cytokines. In some embodiments, incorporating cytokines allows use of the sealants to control or limit adhesion. The tissue sealants are also used as a treatment for ballistic injuries. Internal uses include, but are not limited to, arresting bleeding from an injury to an organ or blood vessel (for example, resulting from blunt abdominal trauma), perioperative bleeding and post-operative hemorrhage. Post surgical examples include, but are not limited to: vascular surgery; cerebrovascular surgery; cardiovascular surgeries such as prosthetic implantation, procedures requiring atrial sutures, aortic dissection, valve repair, septal defect repair, and repairs of vessel or heart chamber rupture; breast reduction, reconstruction, enhancement, or mastectomy; facial surgery such as cosmetic peels (e.g. applying to the location of the peel after the peel is completed), hair transplants and face lifts; placement orthopedic surgery such as knee, hip, spinal, and shoulder repair; neurosurgery (intracranial and spinal surgeries as well as repair of a peripheral nerve), such as duraplasty, dural repair, tumor resection, repair of nerve anastomosis, repair of peripheral nerve, reinforcement of muscular support for cerebral aneurysms, and closing cortical ependymal defects; and dental extractions. Another use is sealing an artery or other tissues or structures that have been punctured or anastamosed as part of a medical procedure such as a biopsy or a catheterization. Sealants at an anastomosis are used, for example, to reattach the vessel to itself or to attach it to a graft. Another use is repairing endoleaks into aneurysms after aneurysm repair by sealing an aneurysm cavity. In some embodiments, the sealants are incorporated into or used with sutures to facilitate wound healing and to provide optimal wound integrity in situations where sutures cannot control, or may aggravate, bleeding. In some embodiments, the sealants are applied preoperatively to help prevent or reduce bleeding during operations, especially in the case of aneurysms or other malformations or weaknesses in a blood vessel or other structure. Placement in some embodiments is aided or guided using radiological techniques.

Tissue sealants may also be used to create an obstruction or reinforcement for an obstruction to a leak of any type to or from any location in the body of an organism. For example, electroprocessed matrices can be used to seal openings in lungs after surgical procedures or injuries involving the lung. The sealants are thus useful as pneumostatics and can prevent, reduce, or eliminate leakage of air. Matrices are also used to seal holes, openings, or defects in membranes such as the peritoneal membrane, the pleural membrane, and the pericardial membrane. This use is important not only for hemostatic purposes but also to prevent air leaking into the pleural cavity and pneumothorax. Another example is use to seal the amniotic sac after amniocentesis. Electroprocessed materials can also be formed in a sleeve to use as reinforcement for aneurysms or at the site of an anastamosis in any vessel, tube or duct. In some embodiments, such sleeves are placed over the area at which reinforcement is desired and sutured, sealed, or otherwise attached to the vessel. Matrices can also be used as plugs for leaks of cerebrospinal fluid, for example after spinal injury, spinal surgery, duraplasty, epidural anesthetic procedures, or other procedures that may lead to leakage. Yet another use is as an obstruction of the punctum lacryma for a patient suffering from dry eye syndrome. Another use is as a fertility control method by injecting a matrix into a duct or tube such as the vas deferens or uterine tube. Many uses combine one or more hemostatic, structural support, or sealant functions, and the description of one or more functions associated with any embodiments herein is not intended to be limiting.

The sealants may also be used for a variety of other functions associated with attachment, providing structural support, or providing a scaffolding for cell, tissue, or organ growth or repair. Examples of urological uses include renal and ureteral sealing, sealing bladder perforations, urethra reconstruction, radical prostatectomy, and partial nephrectomy. Thoracic surgery examples include suture sites, sealant at the site of surgical dissections (including pleurodesis/decortication, tumor resection, and lobectomy/pneumonectomy) treatments of bronchopleural fistulae, pleural adhesions, and pneumothorax, and sealing of a percutaneous lung biopsy. Examples of plastic and reconstructive surgery and otolaryngology includes sealing skin grafts, application as topical bandages, and sealants in face lifts, rhinoplasty, reconstruction of laryngeal structures, scar correction, blepharoplasty, laser surgery, removal of tumors and cysts, surgery in the abdominal area (e.g. “tummy tucks”), hair transplant and other skin flap donor and recipient sites, otoclesis, repair of the tympanic membrane, and repair of the nasal septum. Orthopedic surgery examples include hemostatic functions noted above, and use as a sealant in tendon rupture repair, nerve sealing, repair of osteochondral fractures, bone grafts, replanting cartilage and osteochondral fragments, and fusion of herniated discs. Examples of head, neck, and oral surgery applications include use as a sealant in mandible repair, closure of oral fistulae, repair of facial nerve, repair of hemangiomas, reattaching severed ears, repair of trachea and esophagus; repair of scleral fistula, repair retinal detachment, perforations and eye injuries, and repair of scleral surgical incision. Other surgical uses for the sealants include, but are not limited to, sealing after laporoscopic procedures, sealing biliary radicles and pancreatic bed surgery sealing a bowel anastamosis, sealing pancreatic fistulae from pancreaticoduodenctomy, sealing hepatic ducts and biliary anastamoses, and preoperative portal vein embolization.

Other uses include, but are not limited to, use to manufacture of engineered tissue and organs, including structures such as patches or plugs of tissues or matrix molecules, prosthetics, and other implants, tissue scaffolding devices for use in tissue repair and support such as sutures, surgical and orthopedic screws, and surgical and orthopedic plates, natural coatings or components for synthetic implants, cosmetic implants and supports, repair or structural support for organs or tissues, substance delivery, bioengineering platforms, platforms for testing the effect of substances upon cells, cell culture, and numerous other uses. This discussion of possible uses is not intended to be exhaustive and many other embodiments exist. Furthermore, although many specific examples are provided below regarding combination of electroprocessed materials and/or specific substances, many other combinations of materials and substances may be used. In some embodiments, the sealant is applied to the surface of an object that will be in contact with a location at which hemostasis or some other sealing effect is desired (for example, a medical device that cuts or is inserted into a wound, incision, or other opening in tissue, for example produced by a cannula or the needle of a syringe). In this use, the object applies the sealant to the location, for example upon removal of a needle. Any material that can be electroprocessed onto a device can be deposited in this fashion. Examples include, but are not limited to, electroprocessed material from solutions of PGA, PLA, PGA/PLA combinations, collagen, gelatin, and fibrinogen or combinations thereof. In one embodiment, a ring or similar shape of electroprocessed sealant material is deposited on a portion of the outside surface of a device such as a syringe and the device is configured such that, upon insertion and withdrawal of the device into a tissue, the electroprocessed material remains behind to assist with hemostasis in the site of insertion.

The electroprocessed sealants are also used to support, reinforce, strengthen or connect tissue or structures that have experienced injury, surgery, or deterioration. For example, matrices can be used in a bladder neck suspension procedure for patients suffering from postpartum incontinence. The electroprocessed sealants are used after cosmetic or reconstructive surgery, in some embodiments eliminating the need for sutures or staples. The electroprocessed sealants are used to assist in reattachment of severed body parts such as fingers and toes. Rectal support, vaginal support, hernia patches, and repair of a prolapsed uterus are other illustrative uses. Sealants are also used to close the site of a dissection or resection. The matrices are used to repair or reinforce weakened or dysfunctional sphincter muscles, such as the esophageal sphincter in the case of esophageal reflux. Other examples include reinforcing, acting as fillers, and replacing tissue in vocal cords, epiglottis, thyroid cartilage, and trachea after removal, such as in removal of cancerous tissue.

Compositions for these uses include an electroprocessed agent (such as electroprocessed fibrinogen) alone or may include any other substances or materials. Any substances and materials can be used. Some preferred materials and substances include other proteins and factors in the coagulation cascade (especially thrombin and Factor XIII or XIIIa), anti-fibrinolytic compounds (especially aprotinin and TAFI), antimicrobials, antibacterials, anesthetics, cells, growth factors, anti-inflammatories, and anti-cancer medications. The substances and materials used will depend on the treatment involved. For example, in one embodiment anticancer drugs are placed in a sealant used at the situs of a tumor resection, thus allowing localized rather than systemic delivery. Another example embodiment is use of substances and electroprocessed materials having an antibiotic and anti-inflammatory activity at the location of a skin injury or treatment site for a skin infection.

The sealants may be applied in any form. Some preferred forms include as a sheet or strip for direct application, a component of a bandage or gauze, microdroplets that, for example, form from an electrospray process, a powder or fluff that may be packed or sprinkled onto or into a location of a wound or injury. In some embodiments, electroprocessed materials are ground or milled to produce fine powders which may be used directly or mixed with other agents to produce gels or other material states. In one preferred embodiment, the user has a sheet that can be torn into a desired shape to cover and arrest bleeding in a wound. Another embodiment is a covering, gown, or garment out of the stuff for placement over a site that is at risk to bleed or to become injured (for example, an ulcer, a bedsore, a site of surgery or a location on the skin that may become injured). In that embodiment, the composition does nothing unless bleeding occurs, in which case clots form to provide hemostasis. In one preferred embodiment, sheets are prepared with electrospun fibers aligned such that they will allow the sheets to be readily torn in one direction or so that they will have greater resistance to tearing along a specific axis of dimension. Some embodiments include elastic electrospun materials, for example a sheet of the electroprocessed material that can be stretched over an injury and released, allowing residual tension to pull the open edges of a wound together. In some embodiments, applying an electroprocessed matrix directly to a site in the body of an organism is used to attach or connect tissues in lieu of other connection devices. The ability to prepare different shapes of tissue sealants allows tailoring the application for use. Sheets and patches are used, for example, in some embodiments in which the surface to be sealed has the shape and accessibility to allow placement of a sheet, or where uniformity in size and thickness of the sealant is desired. In some embodiments, sealants are prepared in a form that allows delivery to one or more areas of the respiratory system by inhalation. Examples include, but are not limited to, microdroplets and fine powders. In one embodiment, a highly labile sealant is used to stop bleeding in the respiratory system then quickly cleared to minimize obstruction.

In some embodiments in which the area of application makes application of a sheet not feasible or not desirable, the sealant may be applied in the form of a powder or fluff, or other small particles, or by aerosol or electroprocessed into a wound or surgical field. In some embodiments, endoscopic procedures are used for locations inside the body of an organism. Applicators are also used in some embodiments, either to apply the electroprocessed material or to apply substances to the electroprocessed material after placement. Sealants may also be applied by injection.

In some embodiments, the sealants are combined with substances or electroprocessed materials that provide water absorbency. One example is absorbent polymers, including superabsorbent polymers. Examples of superabsorbents include but are not limited to natural materials such as agar, pectin, carboxyalkyl starch, carboxyalkyl cellulose and guar gum, as well as synthetic materials such as synthetic hydrogel polymers. Examples of synthetic hydrogel polymers include, but are not limited to, carboxymethyl cellulose, alkali metal salts of polyacrylic acid, polyacrylamides, polyvinyl alcohol, hydrolyzed polyacrylonitrile ethylene maleic anhydride copolymers, polyvinyl ethers, hydroxypropyl cellulose, polyvinyl mopholinone, polymers and copolymers of vinyl sulfonic acid, polyacrylates, polyacrylamides, polyvinyl pyrridines, hydrolyzed acrylonitrile grafted starch, acrylic acid grafted starch, and isobutylene maleic anhydride copolymers and mixtures thereof. Partial crosslinking of hydrogel polymers will render the polymers insoluble in water but capable of swelling with water. Superabsorbents can be electroprocessed or combined with the sealant by other means. In some embodiments, these components will serve to absorb liquids that leak from a site to which the sealant is applied and thus reduce the interference by those leaks with the attachment and other functions of the sealant. The absorbent polymers can be electroprocessed or combined with the sealants in any other form.

One preferred electroprocessed sealant composition contains electroprocessed fibrinogen, factor XIII, thrombin, and aprotinin. The fibrinogen is present in the electroprocessed sealant in concentrations between approximately 5 and approximately 2000 mg/ml, preferably between approximately 10 and approximately 1000 mg/ml, more preferably between approximately 50 and approximately 130 mg/ml, even more preferably between approximately 70 and approximately 110 mg/ml. The Factor XIII is present in the electroprocessed sealant in concentrations between approximately 1 and approximately 1000 U/ml, preferably between approximately 5 and approximately 100 U/ml, more preferably between approximately 10 and approximately 80 U/ml, even more preferably between approximately 10 and approximately 50 U/ml. The thrombin is present in the electroprocessed sealant in concentrations greater than zero and up to approximately 7,500 TU/ml, preferably between approximately 100 and approximately 1000 IU/ml, more preferably between approximately 400 and approximately 600 IU/ml, even more preferably approximately 500 IU/ml. The aprotinin is present in the electroprocessed sealant in concentrations between about approximately 100 and about approximately 30,000 KIU/ml, preferably between approximately 500 and approximately 5000 KIU/ml, more preferably between approximately 1000 and approximately 4000 IU/ml, even more preferably approximately 3000 KIU/ml. Each of these components may be electroprocessed into the compositions or combined with the composition by any means. In some preferred embodiments, the concentrations of one or more of these substances are adjusted downward to result in slower hemostasis. In one such embodiment, the thrombin concentration in the electroprocessed sealant is between approximately 0.1 and approximately 100 IU/ml, more preferably between approximately 1 and approximately 10 IU/ml, even more preferably approximately 4 IU/ml. Where the above compositions are combined with collagen, the concentrations of these components are reduced in some embodiments. In one embodiment, the concentrations are reduced 50% in a matrix containing collagen.

The compositions have sufficient density to perform their sealant function. In one embodiment involving electrospun fibrinogen, the density of the electroprocessed material is between approximately 10 and approximately 100 mg/cm³, preferably between approximately 20 and approximately 40 mg/cm³, more preferably approximately 30 mg/cm³. In a variation on this embodiment in which the matrix also contains collagen, the density of the electroprocessed material is reduced by 50%.

### Properties of Sealants Relevant to Uses in Substance Delivery

One use of the electroprocessed sealants of the present invention is the delivery of one or more substances to a desired location. In some embodiments, the sealants are used simply to deliver the electroprocessed materials. In other embodiments, the electroprocessed materials are used to deliver substances that are contained in the electroprocessed materials or that are produced or released by substances contained in the electroprocessed materials. For example, an electroprocessed material containing cells can be implanted in a body and used to deliver molecules produced by the cells after implantation. The present compositions can be used to deliver substances to an in vivo location, an in vitro location, or other locations. The present compositions can be administered to these locations using any method.

In the field of substance delivery, the sealant compositions of the present invention have many attributes that allow delivery of substances using a wide variety of release profiles and release kinetics. For example, selection of the substance and the method by which the substance is combined with the electroprocessed material affects the substance release profile. To the extent that the substances are not immobilized by the electroprocessed material, release from the electroprocessed material is a function of diffusion. An example of such an embodiment is one in which the substance is sprayed into an electroprocessed materials during electroprocessing or onto the electroprocessed material after it has been electroprocessed. In some embodiments in which substances are immobilized by the electroprocessed material, release rate is closely related to the rate at which the electroprocessed material degrades. In other embodiments in which the electroprocessed material is encapsulated, the release rate is tied to dissolution of the encapsulating substance or electroprocessed material. An example of such an embodiment is one in which the substance is covalently bonded to the electroprocessed material. For a substance trapped within an electrospun aggregate or filament, release kinetics are determined by the rate at which the surrounding electroprocessed material degrades or disintegrates. Still other examples are substances that are coupled to the electroprocessed material by a light sensitive bond. Exposing such a bond to light releases the substance from the electroprocessed material. Conversely, in some embodiments of this invention, electroprocessed materials can be exposed to light to cause binding of agents in vivo or in vitro. Combining the compound with the electroprocessed material in solution, rather than in suspension, results in a different pattern of release and thereby provides another level of control for the process. Further, the porosity of the electroprocessed material can be regulated, which affects the release rate of a substance. Enhanced porosity facilitates release. Substance release is also enhanced by milling, fragmenting or pulverizing the electroprocessed material. Pulverized electroprocessed material can, for example be applied to a wound site, ingested or formed into another shape such as a capsule or a tablet. In embodiments in which the substance is present in the form of a large particle such as a tablet encapsulated in the electroprocessed material, or a molecule trapped inside an electroprocessed filament, release is dictated by a complex interplay of the rate the particles dissolve or degrade and any breakdown or degradation of the electroprocessed material structure. In embodiments in which the substance comprises cells that express or produce one or more desired compounds, factors that affect the function and viability of the cells and the timing, intensity, and duration of expression can all affect the release kinetics. Chemicals that affect cell function, such as oligonucleotides, promoters or inhibitors of cell adhesion, hormones, and growth factors, for example, can be incorporated into the electroprocessed material and the release of those substances from the electroprocessed material provides a means of controlling expression or other cellular functions in the electroprocessed material.

Release kinetics in some embodiments are manipulated by cross-linking electroprocessed material through any means. In some embodiments, cross-linking will alter, for example, the rate at which the electroprocessed matrix degrades or the rate at which a compound is released from the electroprocessed material by increasing structural rigidity and delaying subsequent dissolution of the electroprocessed material. Electroprocessed materials can be formed in the presence of cross-linking agents or can be treated with cross-linking agents after electroprocessing. Any technique for cross-linking electroprocessed materials may be used as known to one of ordinary skill in the art. Examples of techniques include but are not limited to application of enzymes or other cross-linking agents and application of certain cross-linking radiations. Examples of cross-linking agents that work with one or more proteins include but are not limited to condensing agents such as aldehydes e.g., glutaraldehyde, carbodiimide EDC (1-ethyl-3(3 dimethyl aminopropyl)), photosensitive agents that cross link upon exposure to specific wavelengths of light, osmium tetroxide, carbodiimide hydrochloride, NHS (n-hydroxysuccinimide), and Factor XIII or XIIIa. Ultraviolet radiation is one example of radiation used to crosslink electroprocessed materials in some embodiments. Electroprocessed natural materials can be cross-linked with other natural substances or electroprocessed natural materials. For example, collagen can be cross-linked and/or stabilized by the addition of fibronectin and or heparin sulfate. For some polymers heat can be used to alter the matrix and crosslink elements of the matrix by fusing adjacent components of the construct. Polymers may also be partially solubilized to alter the structure of the electroprocessed material, for example brief exposure of some synthetics to alcohols or bases can partially dissolve and anneal adjacent filaments together. Some polymers may be cross-linked using chemical fusion or heat fusion techniques. Synthetic polymers generally can be cross-linked using high energy radiation (e.g., electron beams, gamma rays). These typically work by the creation of free radicals on the polymer backbone which then couple, affording cross links. Backbone-free radicals can also be generated via peroxides, azo compounds, aryl ketones and other radical-producing compounds in the presence of heat or light. Reduction-oxidation reactions that produce radicals (e.g., peroxides in the presence of transition metal salts) can also be used. In many cases, functional groups on polymer backbones or side chains can be reacted to form cross-links. For example, polysaccharides can be treated with diacylchlorides to form diester cross-links. Cross-linking may also occur after application of a matrix where desirable. For example, a matrix applied to a wound may be cross-linked after application to enhance adherence of the matrix to the wound.

In some embodiments, an electroprocessed material is processed such that the resulting sealant resembles a solid film. This can be accomplished, for example, by exposing the electroprocessed material to high concentrations of crosslinking agents, increasing the duration of any crosslinking period, or both. Optionally, the electroprocessed material is also exposed to water vapor during this time to cause hydration and swelling of the electroprocessed material to assist in formation of the film. In one embodiment, a sealant is placed in a chamber that also contains a container of 50% solution of glutaraldehyde in water under conditions effect to expose the sealant glutaraldehyde vapor and water vapor. This treatment alters the surface structure of the electrospun fibers and produces a film. Films have many uses. For example, they provide another means of regulating release kinetics of a substance incorporated into the sealant. Films also provide a means for altering the permeability of the sealant to cells and other substances. In one embodiment, a layer of electroprocessed material in a sealant construct is converted to a film to provide an impermeable barrier, for example to reduce or to eliminate evaporative fluid loss from a wound.

In some embodiments, electroprocessed materials that swell upon hydration encapsulate or entrap substances within individual fibers upon swelling. In one embodiment, Type I collagen was electrospun along with the BONE SOURCE product described above. Upon hydration and swelling of the electrospun composition, collagen fibers swelled and entrapped hydroxyapatite crystals within individual fibers.

The release kinetics of the substance is also controlled by manipulating the physical and chemical composition of the electroprocessed materials. For example, small fibers of PGA are more susceptible to hydrolysis than larger diameter fibers of PGA. An agent delivered within an electroprocessed material composed of smaller PGA fibers is released more quickly than when prepared within an electroprocessed material composed of larger diameter PGA fibers.

Release kinetics is also controlled in some embodiments by treating with glue compounds discussed above (e.g. cyanoacrylates). By retarding hydration, swelling, or dissolution of a matrix, these materials slow release profiles in some embodiments.

In some embodiments substances such as peptides can be released in a controlled manner in a localized domain. Examples include embodiments in which the substance is chemically or covalently bonded to the electroprocessed material. The formation of peptide gradients is a critical regulatory component of many biological processes, for example in neovasculogenesis. Physical processing of the formed electroprocessed matrix is another way to manipulate release kinetics. In some embodiments, mechanical forces, such as compression, applied to an electroprocessed material hasten the breakdown of the matrix by altering the crystalline structure of the electroprocessed material. Structure of the matrix is thus another parameter that can be manipulated to affect release kinetics. Polyurethanes and other elastic materials such as poly(ethylene-co-vinyl acetate), silicones, and polydienes (e.g., polyisoprene), polycaprolactone, copolymers of caprolactone with glycolide and/or lactide, poly(hydroxy butyrate) and copolymers, poly(ester-urethanes) and related materials, poly(1,5-dioxepan-2-one) and copolymers, and related polymers are examples of materials whose release rate can be altered by mechanical strain. In some embodiments involving more crystalline polymers (for example, polyglycolic acid and related polymers), application of mechanical tension leads to an increase in crystallinity of the polymer, which will alter the degradation rate, usually by slowing it. Matrices that contain electroprocessed materials that are affected by physical manipulation are thus subject to control by such manipulation.

Release kinetics can also be controlled by preparing laminates comprising layers of electroprocessed materials with different properties and substances. For example, layered structures composed of alternating layers of different electroprocessed materials can be prepared by sequentially electroprocessing different materials onto a target. The outer layers can, for example, be tailored to dissolve faster or slower with respect to the inner layers. Multiple agents can be delivered by this method, optionally at different release rates. Layers can be tailored to provide a complex, multi-kinetic release profile of a single agent over time. Using combinations of the foregoing can provide for release of multiple substances released, each with a complex profile.

Suspending a substance in particles that are incorporated in the electroprocessed materials in the matrix provides another means for controlling release profile. Selection of the composition of these smaller particle matrices provides yet another way to control the release of compounds from the electroprocessed material. The release profile can be tailored by the composition of the electroprocessed material.

Embodiments also exist in which the substances are contained in liposomes or other vesicles such as aggregates of carbohydrates in the electroprocessed matrix. Vesicles are prepared that will release one or more compounds when placed in fluids at a specific pH range, temperature range, or ionic concentration. Methods for preparing such vesicles are known to persons of skill in the art. The electroprocessed material can be delivered to a site of interest immediately or is stored either dry or at a pH at which release will not occur, and then delivered to a location containing liquids that have a pH at which release will occur. An example of this embodiment is an electroprocessed material containing vesicles that release a desired compound at the pH of blood or other fluids released from a wound. The matrix is placed over a wound and releases fluids upon discharge of fluids from the wound.

Incorporating constituents that are magnetically sensitive or electrically sensitive into the electroprocessed materials provides another means of controlling the release profile. A magnetic or electric field is subsequently applied to some or all of the matrix to alter the shape, porosity and/or density of the electroprocessed material. For example, a field can stimulate movement or conformational changes in a matrix due to the movement of magnetically or electrically sensitive particles. Such movement can affect the position of a matrix within a body cavity or the release of compounds from the electroprocessed matrix. For example, altering the conformation of the matrix can increase or decrease the extent to which the electroprocessed material is favorable for compound release.

In some embodiments, magnetically or electrically sensitive constituents that have been processed or co-processed with electroprocessed material are implanted subdermally to allow delivery of a drug over a long interval of time. By passing a magnetic field or an electrical field across the electroprocessed material, drug release is induced. The electroprocessed structure is stable and does not substantially change without electromagnetic stimulation. Such embodiments provide controlled drug delivery over a long period of time. For example, an electroprocessed material that has magnetic or electrical properties and insulin can be fabricated and placed subdermally in an inconspicuous site. By passing a magnetic field or an electrical field across the composition, insulin release is induced. A similar strategy may be used to release compounds from a construct that has light sensitive elements, exposing these electroprocessed materials to light will either cause the electroprocessed material itself to break down and or cause the release of substances that are bound to the electroprocessed material by the light sensitive moiety.

In some embodiments, the substances comprise vesicles encapsulated within the electroprocessed material along with electrical or magnetic substances or electroprocessed material. The vesicles contain a compound to be released from the vesicles. Placing an electrical or magnetic field across the electroprocessed material causes the compounds within the vesicles to be released by, for example, deforming the vesicles to the point of rupture or by changing the permeability (in some cases reversibly) of the vesicle wall. Examples of these embodiments include transfection agents, such as liposomes, that contain nucleic acids that enhance the efficiency of the process of gene delivery to cells.

In some embodiments, the composition comprising electroprocessed material and substances is used as a transdermal patch for localized delivery of medication, or of a component of such a patch. In some of these embodiments, electrically conductive substances or electroprocessed materials are incorporated into such a composition, which is then used as a component of an iontophoresis system in which one or more substances is delivered in response to the passage of electric current. Electrically conductive compounds and piezoelectric crystals are examples of electroprocessed materials and substances that can have a direct healing effect on bone injuries. For example placing a small electric current across a fracture site promotes healing. An electroprocessed bone mimetic that conducts or produces current can be made and placed within a fracture. The addition of the electrical current promotes healing at a rate that is faster than the addition of the electroprocessed composition alone.

In other embodiments, an electroprocessed material or a portion thereof containing electromagnetic properties is stimulated by exposure to a magnet to move and thereby apply or release physical pressure to a pressure-sensitive capsule or other enclosure that contains molecules to be released from the electroprocessed material. Depending on the embodiment, the movement will affect the release relate of the encapsulated molecules.

Response of the composition to electric and magnetic fields can be regulated by features such as the composition of the electroprocessed materials, size of the filaments, and the amount of conductive compounds added. Electromechanical response from polyaniline is the result of doping-induced volume changes, whereas ion gradients leading osmotic pressure gradients are responsible for field-induced deformation in ionic gels such as poly(2-acrylamido-2-methyl propanesulfonicacid). In each case, ion transport kinetics dominate the response, and facile transport is observed with the small fibers. Gel swelling and shrinking kinetics have been shown to be proportional to the square of the diameter of a gel fiber. Electromechanical response times of fiber bundles of less than 0.1 s, are possible in typical muscle.

Embodiments involving delivery of molecules produced by cells provide many means by which rejection and immune response to cells can be avoided. Embodiments using cells from a recipient thus avoid the problems associated with rejection and inflammatory and immunological responses to the cells. In embodiments in which cells from an organism other than the recipient are used, the matrix can sequester the cells from immune surveillance by the recipient's immune system. By controlling parameters such as the pore size or chemical composition of the electroprocessed material, nutritive support to the cells trapped in the matrix can be permitted while the cells are protected from detection and response by the recipient's immune system. As an example, pancreatic islet cells that manufacture insulin collected from a donor can be encapsulated in an electroprocessed matrix and implanted in a recipient who cannot make insulin. Such an implant can be placed, for example, subdermally, within the liver, or intramuscularly. For some immune responses permanent sequestration from the host system may not be necessary. The electroprocessed material can be designed to shield the implanted electroprocessed material for a given length of time and then begin to breakdown. In still other embodiments, bacteria or other microbial agents engineered to manufacture the desired compound can be used. This embodiment provides the advantages of using cells that are more easily manipulated than cells from the recipient or a donor. Again, the electroprocessed material can serve to shield the bacteria from immune response in this embodiment. The advantage of using a bacterial carrier is that these microbes are more easily manipulated to express a wide variety of products. Embodiments in which cells are transiently transfected allow for expression to be limited to a defined period. Transient genetic engineering allows cells to revert to their original state in embodiments in which such reversion is desired to minimize the risks of complications.

In some embodiments, cells are genetically engineered such that the expression of a specific gene may be promoted or inhibited through various means known in the art. For example, a tetracycline sensitive promoter can be engineered into a gene sequence. That sequence is not expressed until the tetracycline is present. Cell markers or bacterial markers can also be used to identify the inserted electroprocessed material. For example, green fluorescent proteins placed within an engineered genetic material glow green when expressed. Embodiments using this feature allow verification of the viability of the cells, bacteria, or gene sequences in a matrix. The visibility of such a marker also assists in recovering an implanted electroprocessed composition.

Although the present invention provides versatility in release kinetics, embodiments also exist in which one or more substances are not released from the electroprocessed material. Substances may perform a function at a desired site. For example, in some embodiments, antibodies specific for a molecule are immobilized on an electroprocessed matrix and the composition is placed at a desired site. In this embodiment, the antibodies act to bind these molecules in the vicinity of the composition. This embodiment is useful for isolating molecules that bind to an antibody. An example is an electroprocessed matrix containing immobilized substrates that will bind irreversibly to an undesirable enzyme and thereby inactivate the enzyme. In another embodiment, substances that are immobilized on an electroprocessed matrix will stimulate a cellular response when a cell comes in contact with the substances. One example is a growth factor covalently linked to the matrix in such a way that it will not be released but will stimulate a cellular response when cells come in contact with the immobilized growth factor.

### Stability and Storage of the Sealants

The stability of the tissue sealants of the present invention allows for long term storage of the sealants between manufacture and use. Stability allows greater flexibility for the user in embodiments in which a substance is applied after formation of the electroprocessed material, for example by soaking and spraying. A formed electroprocessed matrix can be fabricated and stored, and then the exact substance composition to be added in a specific application can be prepared and tailored to a specific need shortly before implantation or application. This feature allows users greater flexibility in both treatment options and inventory management. In many embodiments, electroprocessed material is essentially dry once it is electroprocessed, thereby facilitating storage in a dry or frozen state. Further, the electroprocessed compositions are substantially sterile upon completion, thereby providing an additional advantage in therapeutic and cosmetic applications. Electroprocessed materials in some embodiments are also substantially dry, thus allowing factors in the coagulation cascade to be combined and stored in a single packaging without premature clotting that could render the sealant useless. This is advantageous as compared to other sealants in which factors must be stored separately or in liquid form.

Storage conditions for the tissue sealants of the present invention will depend on the electroprocessed materials and substances therein. In some embodiments involving proteins, for example, it may be necessary or desirable to store the compositions at temperatures below 0° C., under vacuum, or in a lyophilized condition. Other storage conditions can be used, for example, at room temperature, in darkness, in vacuum or under reduced pressure, under inert atmospheres, at refrigerator temperature, in aqueous or other liquid solutions, or in powdered form. In some embodiments, the sealants are stored in a dessicated state. Dessicated sealants are optionally packaged with desiccants, such as silica gel, to maintain dessication. Persons of ordinary skill in the art recognize appropriate storage conditions for the electroprocessed materials and substances contained in the compositions and will be able to select appropriate storage conditions.

The tissue sealants of the present invention and formulations comprising those compositions may be sterilized through conventional means known to one of ordinary skill in the art. Such means include, but are not limited to, filtration, radiation, and exposure to sterilizing chemical agents such as peracetic acid or ethylene oxide gas. Heat may also be used in embodiments in which the application of heat will not substantially denature electroprocessed natural materials or substances in the compositions. The compositions of the present invention may also be combined with bacteriostatic agents, such as thimerosal or compositions of oligodynamic metals such as silver to inhibit bacterial growth.

Formulations comprising the electroprocessed compositions of the present invention may be presented in unit-dose or multi-dose containers, for example, sealed ampules and vials, and may be stored in a freeze-dried (lyophilized) condition requiring only the addition of the sterile liquid carrier, for example, water for injections or other modes of application, immediately prior to use. Extemporaneous injection solutions and suspensions may be prepared from sterile powders, granules and tablets commonly used by one of ordinary skill in the art. Other embodiments involve electroprocessed matrices in a sheet serving as a bandage or otherwise packaged for easy use. Preferred unit dosage formulations are those containing a dose or unit, or an appropriate fraction thereof, of the administered ingredient. It should be understood that in addition to the ingredients particularly mentioned above, the formulations of the present invention may include other agents commonly used by one of ordinary skill in the art.

The electroprocessed compositions of the present invention may be packaged in a variety of ways depending upon the method used for administering the composition. Generally, an article for distribution includes a container which contains the composition or a formulation comprising the composition in an appropriate form. Suitable containers are well-known to those skilled in the art and include articles such as bottles (plastic and glass), sachets, ampules, paper bags or packets, plastic bags, metal cylinders, and the like. The container may also include a tamper-proof assemblage to prevent indiscreet access to the contents of the package. In addition, the container has deposited thereon a label which describes the contents of the container. The label may also include appropriate warnings.

The ability to store the electroprocessed materials for an extended period provides the ability to isolate electroprocessed materials and substances for preparing the compositions from the patient for a period as long as years in advance of use. This allows subsequent use of autologous material and reduces risks of immunological responses and viral and other infections that can be associated with heterologous material.

Other Properties of the Electroprocessed Sealant Compositions

The sealant compositions of the present inventions have a number of beneficial properties. The following are examples of properties of certain embodiments. The list is not exhaustive of the properties. Embodiments exist that do not have the properties discussed below. Embodiments also exist that have any combination of these properties. In some embodiments, the electroprocessed sealants form a matrix. Some such matrices are similar to extracellular matrices. Many of the properties discussed below relate to properties of specific matrices. The tissue sealants of the present invention include sealants contain matrices formed by electroprocessing. Wherever matrices are discussed, it is to be understood that such matrices are components of tissue sealants of the present invention. Embodiments also exist in which the sealants are not in the form of a matrix.

Some embodiments have hemostatic properties. Examples include, but are not limited to embodiments that contain one or more of the following: electroprocessed fibrin, fibrinogen, thrombin, and other proteins or factors that are part of a coagulation cascade, as well as mimetics for such proteins or factors; collagen; synthetic polymers such as PGA, PLA, and PGA/PLA copolymers; synthetic polymers having cationic moieties; gelatin; and certain carbohydrates such as chitosan and alginate salts such as calcium alginate and sodium alginate. Embodiments exist that have varying speeds of hemostasis, thus allowing preparation of compositions that cause hemostasis at a desired speed. For example, in some embodiments the use of electroprocessed materials that have a higher solubility in tissue fluids, use of higher concentrations of electroprocessed materials or substances that promote coagulation (e.g. thrombin), and, when electrospun fibers are used, use of fibers having a smaller diameter are ways to increase the speed of hemostasis. Applying the opposite of these characteristics has the opposite effect (i.e. decreasing speed of hemostasis) in some embodiments. Encapsulating substances that promote hemostasis is another way of reducing the speed of hemostasis in some embodiments. In some embodiments, hemostasis occurs quickly enough that the sealant may be applied to a high volume bleed in a surgical field (such as a punctured spleen, liver, or artery) for a brief period, then removed without further bleeding. In some embodiments the period is less than 30 minutes. In other embodiments, the period is less than ten minutes. In other embodiments, the period is less than five minutes. In other embodiments, the period is less than one minute. This property can be beneficial in surgical applications because it allows reduction of the size or amount of the implant left within the body of patient after surgery.

In many embodiments, use of the sealants of the present invention helps reduce the degree of adhesion (formation of scar tissue) in the location of use. This property is advantageous, for example, in uses in which scar tissue formation can be problematic, such as obstetric procedures, cosmetic surgery, gastrointestinal surgery, cardiovascular applications in which there is a risk that scar tissue will weaken a blood vessel or cardiac tissue.

In some embodiments, the electroprocessed tissue sealants have a translucent or even transparent appearance or will become transparent or translucent when wetted. This property allows visual inspection of the underlying tissue, an advantage in, for example, brain surgery and other neurosurgery, sinus surgery, and procedures in other areas adjacent to vascular beds or to the brain.

In some embodiments, electrospun materials suppress or promote the activation of matrix metalloproteinases (MMPs), a protein that is often overexpressed in wounds. Some embodiments of electrospun collagen will suppress activation of MMPs. Some embodiments using electrospun gelatin will promote activation of MMPs.

In some embodiments the tissue sealant is used as an implant within or replacement of tissues or organs of the body of an organism or as a part of such an implant or replacement. In some embodiments, the tissue sealants form a matrix, in some cases a matrix similar to an extracellular matrix. For example, the type of electroprocessed material selected can be based on the similarity to tissue in which the composition will be implanted, or, in the case of a prosthetic, the type of tissue, structure, or organ being replaced, repaired, or augmented. In such embodiments, the electroprocessed material is combined with extracellular matrix materials to more closely mimic tissues. Such combination can occur before, during, or after formation of the matrix. Some extracellular materials are electroprocessed into a matrix or formed through other means. In some embodiments matrix materials are added to electroprocessed material once the matrix has been fabricated.

The electroprocessed compositions used in the tissue sealants of the present invention have many features that make them suitable for formation of extracellular matrices. The fibril structure and banding of many electrospun materials (including but not limited to some electrospun collagens or fibrinogen) is similar to that of naturally occurring molecules. The density and structure of matrices formed by this method are greater than those achieved by known methods and are more similar to that of natural extracellular matrices.

In some embodiments involving electrospinning, fibers are produced with much lower diameters than those that can be produced by known manufacturing processes. Electrospun collagen and fibrinogen have been observed to have cross-sectional diameters ranging from several μm down to below 100 nm. Electrospun fiber diameter can be manipulated by changing, for example, the composition (both in terms of sources and types of materials) and concentration of materials to be electrospun. In some embodiments, fiber diameter increases linearly with concentration. In some embodiments, fiber diameter in an electrospun preparation becomes more disperse or varied with an increase in concentration. In some embodiments, the addition and removal of molecules that regulate or affect fiber formation can be added to manipulate fiber formation. Many proteoglycans, for example, are known to regulate fiber formation, including affecting the diameter of fibers. While specific ranges have been disclosed herein in discussing the characteristics of examples of electroprocessed materials sealants, it is to be understood that such ranges are not intended to be limiting. For example, a wide range of fiber diameters for electroprocessed fibers are achievable, ranging from in excess of 10 μm to below 80 nm. The invention includes fibers within these ranges wherein the fibers comprise any type of electroprocessed material, including natural materials and synthetic polymers, and combinations thereof. Examples of fibers electrospun from fibrinogen solutions include, but are not limited to: fibers with average diameters ranging from about 80-700 nm, fibers with an average diameter between about 82 and 91 nm; and fibers having average diameters of any of 80±20 nm, 310±70 nm and 700±110 nm. Examples with collagen include, but are not limited to: Type I collagen with individual filament diameters ranging from about 100-730 nm; Type I collagen fibers with an average diameter of 100±40 nm; Type II collagen fibers with an average diameter of about 1.0 μm; Type II collagen fibers with an average diameter of about 3±2.5 μm; Type II collagen fibers with an average diameter of about 1.75±0.9 μm; Type II collagen fibers with an average diameter of about 110±90 nm; Type III collagen fibers with average diameters of about 250±150 nm; an electrospun blend of Type I and Type III collagen fibers with an average diameter of about 390±290 nm; and blends of Type I collagen/Type III collagen/elastin (45:35:20 or 40:30:20) having a diameter of about 800±700 nm. Ranges of larger fiber sizes are also possible. In one desirable embodiment, the electroprocessed fibers range between about 10 nm and 100 μm in average diameter. In another desirable embodiment, the fibers range between about 50 nm and about 10 μm in average diameter. In another desirable embodiment, the fibers range between about 70 nm and about 10 μm in average diameter. In another desirable embodiment, the fibers range between about 50 nm and about 1 μM in average diameter. In another desirable embodiment, the fibers range between about 70 nm and about 1 μm in average diameter. In another desirable embodiment, the fibers range between about 100 nm and 1 μm in average diameter. In one preferred embodiment, the diameters of the electroprocessed material are similar to that of extracellular matrix materials in vivo. The foregoing discussion regarding possible fiber diameter ranges is not limited to electrospun collagen or fibrinogen, or to specific types of these proteins, but applies to all types of electroprocessed materials, including electrospun collagen, fibrinogen, fibrin, fibronectin, chitin, chitosan, any other types of natural materials, and any types of synthetic materials. It is to be understood that the invention includes electroprocessed materials of any diameter, and that none of the above diameters is intended to be limiting. Examples of preferred embodiments involving electrospun collagen of a specific type and specific diameter include, but are not limited to: electrospun Type I collagen fibers with an average diameter between about 50 nm and about 10 μM, more preferably between about 50 nm and about 1 μm; electrospun Type II collagen fibers within an average fiber diameter between about 10 and about 80 nm; electrospun Type III collagen fibers within an average fiber diameter between about 30 nm and about 150 nm. One preferred embodiment with electrospun fibrinogen has a diameter between about 50 nm and about 150 nm, more preferable between about 80 nm and about 95 nm. In many embodiments, the electrospun material forms as a continuous fiber such that spun materials show no evidence of free ends upon microscopic examination. Other embodiments do not involve such formation of a continuous fiber.

The present invention permits design and control of pore size in an electroprocessed material through manipulation of the composition of the electroprocessed material and the parameters of electroprocessing. In some embodiments, the sealant matrix has a pore size that is small enough to be impermeable to one or more types of cells. In some embodiments, the sealant is a film having no measurable pore dimension. In some embodiments in which the sealant is used as a hemostatic agent, for example, the pore size is such that the sealant is impermeable to red blood cells. In some embodiments, the pore size is such that the sealant is impermeable to platelets. In one embodiment, the average pore diameter is about 500 nm or less. In another embodiment, the average pore diameter is about 1 μm or less. In another embodiment, the average pore diameter is about 2 μm or less. In another embodiment, the average pore diameter is about 5 μm or less. In another embodiment, the average pore diameter is about 8 μm or less. In some embodiments, the pore size is large enough to allow some penetration and fragmentation to initiate clotting. Some embodiments have pore sizes that do not impede cell infiltration at all. One preferred embodiment has a pore size between about 0.1 μm² and about 100 μm². A further preferred embodiment has a pore size between about 0.1 μm² and about 50 μm². A further preferred embodiment has a pore size between about 1.0 μm² and about 25 μm². A further preferred embodiment has a pore size between about 1.0 μm² and about 5 μm². Infiltration can also be accomplished with implants with smaller pore sizes. In other embodiments, the use of electrospun matrices in implants promotes cellular infiltration of the implants. In fact, some constructs comprising matrices of the present invention display a propensity for cellular migration not previously known to be achievable by implanted constructs. For porous structures, the interaction of the electroprocessed material with the host surrounding tissue is dependent on the size, size distribution, and continuity of pores within the structure of the device. It was previously thought that pore size must be greater than about 10 μm for cells to be capable of migrating into, out of, or through the structure. It has been observed, however, that implants comprised of electrospun nanofibers of at least some types of natural proteins are not subject to this limitation. In one embodiment significant cellular migration occurred into an electrospun collagen/elastin with an average pore diameter of 3.7 μm. Pore size of an electroprocessed matrix can be readily manipulated through control of process parameters, for example by controlling fiber deposition rate through electric field strength and mandrel motion, by varying solution concentration (and thus fiber size). Porosity can also be manipulated by mixing porogenic agents, such as salts or other extractable agents, the dissolution of which will leave holes of defined sizes in the matrix. If desired, the degree to which cells infiltrate a matrix can be controlled by the amount of cross-linking present in the matrix. A highly cross-linked matrix is not as rapidly infiltrated as a matrix with a low degree of cross-linking. Adding electroprocessed synthetic materials to a matrix also limit the degree to which cells infiltrate the electroprocessed material in some embodiments. Cell infiltration is also limited in some embodiments by incorporating agents that act to actively suppress cell migration (for example, cell toxins such as sodium azide, bacterial toxins or certain pharmaceuticals).

Electroprocessed sealant matrices also include some embodiments with high surface area to volume ratio as well as high surface area to weight ratios. With respect to surface area to volume, in one embodiment, the surface area to volume ratio is greater than about 1000 cm²/cm³. In some embodiments, the ratio is about 100 cm²/cm³ or higher. In another embodiment, the surface area to volume ratio is greater than about 10,000 cm²/cm³. In another embodiment, the surface area to volume ratio is greater than about 50,000 cm²/cm³. In another embodiment, the surface area to volume ratio is greater than about 100,000 cm²/cm³. In another embodiment, the surface area to volume ratio is greater than about 250,000 cm²/cm³. In another embodiment, the surface area to volume ratio is between about 4,000 cm²/cm³ and about 400,000 cm²/cm³. In another embodiment, the surface area to volume ratio is between 50,000 cm²/cm³ and 200,000 cm²/cm³. In some embodiments, the ratio is between about 100 cm²/cm³ and about 10,000 cm²/cm³. In other embodiments, the ratio is between about 1000 cm²/cm³ and about 5000 cm²/cm³. In other embodiments, the ratio is between about 5000 cm²/cm³ and about 10000 cm²/cm³. In other embodiments, the ratio is between about 6500 cm²/cm³ and about 8000 cm²/cm³. In other embodiments, the ratio is about 7200 cm²/cm³. In other embodiments, the ratio is between about 3000 cm²/cm³ and about 4000 cm²/cm³. In other embodiments, the ratio is about 3300 cm²/cm³.

With respect to surface area to weight ratios, in one embodiment, the surface area to weight ratio is greater than about 0.50 m²/g. In another embodiment, the surface area to weight ratio is greater than about 1.00 m²/g. In another embodiment, the surface area to weight ratio is greater than about 5.00 m²/g. In another embodiment, the surface area to weight ratio is greater than about 10.00 m²/g. In another embodiment, the surface area to weight ratio is greater than about 25.00 m²/g. In another embodiment, the surface area to weight ratio is greater than about 50.00 m²/g. In one embodiment, the surface area to weight ratio is between about 0.5 m²/g and about 55 m²/g. In another embodiment, the surface area to weight ratio is between about 7 m²/g and about 28 m²/g. In some embodiments, the ratio is between about 1 m²/g and about 10 m²/g. In other embodiments, the ratio is between about 1 m²/g and about 5 m²/g. In other embodiments, the ratio is between about 5 m²/g and about 10 m²/g. In other embodiments, the ratio is between about 3.5 m²/g and about 5 m²/g. In other embodiments, the ratio is about 4.1 m²/g. In other embodiments, the ratio is between about 8 and about 10 m²/g. In other embodiments, the ratio is about 9 m²/g.

Electroprocessed sealant matrices have the advantage of greater structural strength than many known sealants, and of retaining that structural strength after implantation. In some embodiments, electroprocessed matrices have greater structural integrity than, for example, the fibrin and collagen gels used in current sealants. Many sealants have such low structural strength that pressure cannot be applied to the sealants to assist attachment or hemostasis because the pressure will deform the sealant structure or flow the sealant away from the site of application. Many embodiments of electroprocessed sealants have sufficient structural strength that they substantially hold their shape under moderate pressure. In some embodiments, electroprocessed fibrinogen is insoluble in water, thus reducing loss of strength due to dissolution. This structural strength also allows the sealants of the present invention to resist being washed away from a site of application by a flow of blood or other fluids. In one embodiment, vigorous blood flow due to the puncture of an abdominal aorta did not wash away a sheet of electroprocessed fibrinogen. In some embodiments, the strength of the sealant is sufficient to allow repositioning the sealant after initial application, even after a portion of the sealant has become wet with blood or other fluids. Another problem that can occur with hemostatic agent or sealants in a liquid, gel, or semisolid state is the tendency for a gauze or bandage backing to absorb those sealants when pressure is applied. When this occurs, the sealant or hemostatic agent may adhere to the gauze or bandage and pull away from a wound or other site of application. In some embodiments, the sealants of the present invention remain sufficiently solid that they are not absorbed or otherwise attached to a bandage or gauze and thus do not pull away from a wound or other site of application when a bandage, gauze, or other backing is removed. The invention is not limited to solids and some embodiment have a consistency similar to that of a gel. In some embodiments, the sealants show less susceptibility to reformation and resorption after implantation than known technologies for making sealants. The present invention also includes methods of controlling the degree to which the electroprocessed materials will be resorbed. In some embodiments, electrospun materials can be resorbed quickly, in a period of 7-10 days or shorter. In other embodiments, a feature such as extensive cross-linking is used to make the matrix very stable to last months to years. Variation of crosslinking also provides a further ability to mimic natural tissue. Natural structural proteins within the body exhibit differing degrees of cross-linking and biological stability. The degree of cross-linking in native proteins may vary as a function of age, physiological status and in response to various disease processes. Any combination of properties for increasing or decreasing strength may be used. Some embodiments involve formation of sheets having relatively uniform thickness, thus providing for uniform strength throughout a sealant structure. However, in other embodiments the thickness, composition, or both of the sealant are varied using factors discussed elsewhere herein.

Embodiments also exist in which the sealants have varying degrees of elasticity. Elasticity is controlled in some embodiments through the selection of materials to be electroprocessed. Use of Type I collagen or PLA, for example, tends to decrease elasticity. Examples of electroprocessed materials that tend to increase elasticity include, but are not limited to, electroprocessed Type III collagen, elastin, polyurethane, poly(ethylene-co-vinyl acetate), silicones, polydienes (e.g., polyisoprene), caprolactone, copolymers of caprolactone with glycolide and/or lactide, poly(hydroxy butyrate) and copolymers thereof, poly(ester-urethanes) and related materials, and poly(1,5-dioxepan-2-one) and copolymers, thereof. Thus, embodiments include, for example, a highly flexible sealant or matrix placed on an injury site on the liver, a firmer, stiffer sealant or matrix used with bone injuries, and matrices containing a large amount of collagen for skin. Elasticity is also decreased in some embodiments by increasing the degree of crosslinking. Formation of thicker structures also serves to increase elasticity. In some embodiments, elasticity is decreased by increasing alignment of electrospun fibers or by increasing the degree of crosslinking in the electroprocessed material.

Combined electroprocessed compositions containing a variety of electroprocessed materials may be prepared for use in the sealants. Compositions can be tailored to mimic the extracellular matrix. In some embodiments, electroprocessed material includes electroprocessed collagen, fibrinogen, fibrin, elastin, laminin, fibronectin, integrin, hyaluronic acid, chondroitin 4-sulfate, chondroitin 6-sulfate, dermatan sulfate, heparin sulfate, heparin, keratan sulfate, or proteoglycans or combinations thereof in appropriate relative amounts to mimic the composition of extracellular matrix materials. Where appropriate, substances comprising extracellular materials can be prepared by means other than electroprocessing and combined with the electroprocessed material. In some embodiments, crude extracts of proteins isolated from the connective tissues are electroprocessed. In such embodiments, the matrix contains a variety of structural and regulatory elements that may be needed to promote activities such as healing, regeneration, and cell differentiation.

Other electroprocessed materials can be included in the sealant matrix to provide other properties. One example is the ability to control the persistence or biodegradation of the implanted matrix. In some embodiments, electroprocessed fibrin tends to degrade faster than electroprocessed collagen when implanted, while some electroprocessed synthetic polymers tend to degrade more slowly. Controlling the relative content of these electroprocessed materials affects the rate at which the matrix degrades. As another example, electroprocessed materials may be included to increase the susceptibility of a matrix or construct formed from a matrix to heat sealing, chemical sealing, and application of mechanical pressure or a combination thereof. It has been observed that inclusion of synthetic polymers (for example, the addition of PGA in an amount of 20% of total electroprocessed material) enhances the ability of matrices to be cauterized or heat sealed. The inclusion of electrically or magnetically reactive polymers in electroprocessed materials is another example. In some embodiments, such polymers are used to prepare matrices that are conductive, that provide a piezoelectric effect, or that alter the shape, porosity and/or density of the electroprocessed material in response to an electric or magnetic field.

The ability to incorporate substances into an electroprocessed tissue sealant allows for additional benefits. One such benefit is even closer mimicry of tissue where desired and greater compatibility where used in or with implants. In some preferred embodiments, stem cells, committed stem cells that will differentiate into the desired cell type, or differentiated cells of the desired type, are incorporated to more closely mimic tissue. Furthermore, the methods available for encapsulating or otherwise combining cells with electroprocessed material leads to greater cell density in the matrix than that achievable by known methods. In some embodiments, this density is enhanced further by the improved cellular infiltration discussed above.

The ability of sealants of the present invention to mimic natural molecules and compositions minimizes the risk of immune rejection of the sealants. For example, autologous material can be used. However, the close resemblance of the electroprocessed materials to natural materials has allowed avoidance of immune reaction even in some embodiments in which heterologous materials are used. For example, electrospun cylinders of bovine Type I collagen (25 mm long by 2 mm wide) implanted into the rat vastus lateralis muscle showed no immune response after 7-10 days. Similar constructs composed of electrospun Type I collagen were supplemented with satellite muscle cells (myoblasts) and implanted. Similar results occurred, there was no evidence of inflammation or rejection and the implants were densely populated. Furthermore, some embodiments of matrices comprising electroprocessed materials have been observed to avoid encapsulation of implants by recipient tissue, a common problem with implants. In embodiments in which encapsulation is desired, matrix structure is altered to promote inflammation and encapsulation.

Substances that can provide favorable sealant characteristics also include drugs and other substances that can produce a therapeutic or other physiological effect on cells and tissues within or surrounding an implant. Any substance may be used. In many preferred embodiments, substances are included in the electroprocessed sealant matrix that improve the performance of the implanted electroprocessed matrix. Examples of substances that are used include but are not limited to peptide growth factors, antibiotics, anesthetics, and anti-rejection drugs, as well as combinations of one or more of the foregoing. Chemicals that affect cell function, such as oligonucleotides, promoters or inhibitors of cell adhesion, promoters and inhibitors of cell intracellular signal cascades, hormones, and growth factors are additional examples of substances that can be incorporated into the electroprocessed material and the release of those substances from the electroprocessed material can provide a means of controlling expression of genes or other functions of cells in the electroprocessed material. Alternatively, cells that are engineered to manufacture desired compounds can be included. The entire construct is, for example, cultured in a bioreactor or conventional culture or placed directly in vivo. For example, neovascularization can be stimulated by angiogenic and growth-promoting factors, administered as peptides, proteins or as gene therapy. Angiogenic agents can be incorporated into the electroprocessed matrix. Alternatively, where neovascularization is not desired, antiangiogenic substances, such as angiostatin, may be included in the electroprocessed matrix. Nerve growth factors can be electrospun into the electroprocessed matrix to promote growth of neurons into the matrix and tissue. In a degradable electroprocessed matrix, the gradual degradation/breakdown of the matrix will release these factors and accelerate growth of desired tissues. Substances can be incorporated into the electroprocessed matrix to regulate differentiation of cells in the matrix. Oligonucleotides, peptides, and drugs such as retinoic acid are examples of such substances. Oligonucleotide DNA or messenger RNA sequences coding for specific proteins in the sense and antisense direction can also be used. For example, where expression of a protein is desired, sense oligonucleotides can be provided for uptake by cells and expression. Antisense oligonucleotides can be released, for example, to suppress the expression of gene sequences of interest. Implants can be designed such that the substances affect cells contained within the matrix, outside the matrix or both.

Several methods exist for studying and quantifying specific characteristics of the electroprocessed materials in the sealants of the present invention. The fiber diameter and pore dimensions (porosity) for matrices can be determined, for example, by SEM micrograph that are digitized and analyzed with UTHSCSA ImageTool 2.0 (NIH Shareware). Water permeability, a characteristic that differs from porosity, may also be studied using standard methods. Atomic force microscopy can also be used to prepare three-dimensional images of surface topography of biological specimens in ambient liquid or gas environments and over a large range of temperatures. This tool allows determination of relationship and interaction between matrix components. Construct composition analysis can include, for example, histological analysis to determine the degree of cellular distribution in the constructs' interstitial spaces. To perform this analysis, cells may be stained with any known cell staining technique (for example, hematoxylin and eosin and Masson's trichrome). Proliferative activity of cells can be studied, for example, by labeling cells biosynthetically with a label that is incorporated into cells actively undergoing DNA synthesis (for example, with bromodeoxyuridine) and using antibodies to determine the extent to which cells are undergoing nuclear division. Cellular density may be determined, for example, by measuring the amount of DNA in enzyme-digested samples utilizing known techniques. Degree of degradation or remodeling of the matrix by cells may be determined by, for example, measuring expression and activity of matrix metalloproteinases from cells. The functionality of cells in electroprocessed matrices is determined by measuring various physiological markers characteristic of the tissues. For example, muscle cells may be stimulated with an electrical signal or challenged with chemical agents or drugs, for example carbachol, to determine the contractility of a construct. Function of cells in an endocrine construct can be determined by measuring production of hormones. One skilled in the art will understand that the foregoing list is not exhaustive and numerous parameters can be used to characterize tissues and matrices using existing methods.

In some embodiments, the sealants induce, promote, inhibit, regulate, or otherwise affect a biological activity. Examples disclosed herein include inducing hemostasis and inducing cell migration by chondrocytes. However, methods of affecting any type of biological activity are within this invention. Activities can be affected by, for example, contacting the cells with a matrix comprising electroprocessed material. “Contacting” the cells with the matrix can be accomplished by any means of placing the cells in close proximity to the matrix including, but not limited to, seeding the cells upon matrix, applying the cells to the matrix by spraying or dripping the cells onto the matrix or the electroprocessing target, electroprocessing the cells, and applying the matrix to existing tissues or other preparations of cells. The invention thus includes methods of promoting, inhibiting, regulating, or otherwise affect a biological activity using electroprocessed materials, either alone or with substances.

### Shapes of Electroprocessed Materials and Matrices

The present invention also provides an electroprocessed material having a predetermined shape, as well as methods for making those shaped electroprocessed materials. In some embodiments the electroprocessed material is made by pre-selecting a mold or mandrel adapted to make the predetermined shape wherein the mold comprises a grounded target substrate and the shape of the matrix is dictated by the outer dimensions of the mandrel. Then, one or more electroprocessed materials are streamed onto the grounded target substrate under conditions effective to deposit the desired electroprocessed materials on the substrate to form the extracellular matrix having the predetermined shape. In some embodiments, a shape is reproduced and created inside a mold designed to mimic that shape. The mold is then be filled by electroprocessing the materials into the mold. In this way, the shape of the matrix mimics the mold shape. The electroprocessed material streamed onto the substrate may comprise electrospun fibers, electroaerosol droplets, electroprocessed powders or particles, or a combination thereof. The formed matrix having a shape of the substrate is then allowed to cure and removed from the mandrel or mold. In some embodiments, the sealant matrix is formed on a moving conveyor or other moving substrate such that a continuous matrix, for example in the form of a continuous sheet, is made.

Electroprocessing allows great flexibility and makes it possible to customize the sealant to virtually any shape needed. Some preferred examples include a flattened oval or circular shape, a rectangular envelope shape, a sheet, a ribbon, a cylinder, a plug to insert into a penetrating injury, a sleeve for placing around a vessel or duct, a nerve guide, skin or muscle patch, a dural patch, a powder, a fluff or batt, a bandage or gauze pad, a fascial sheath, vertebral disc, articular cartilage, knee meniscus, ligament, tendon, or a vascular graft for subsequent use in vivo. In some embodiments, electrospun fibers are aligned along a specific axis or dimension of the shape, making the resulting matrix amenable to tearing along that axis or dimension. This alignment allows the user to tear off strips of an electroprocessed material, for example to be used as a bandage. The matrix can be shaped to fit a defect or site to be filled, such as a site where a tumor has been removed, or an injury site in the skin (a cut, a biopsy site, a hole or other defect) or the location of a missing or shattered piece of bone. A particular type of organ or tissue that is desired to be made or replaced has a specific shape, such as a skin patch to fit a biopsy site or a large scalp area removed after discovering a malignant melanoma. The electroprocessed compositions may be shaped into shapes useful for substance delivery, for example, a skin patch, a lozenge for ingestion, an intraperitoneal implant, a subdermal implant, the interior or exterior lining of a stent, a cardiovascular valve, a tendon, a cornea, a ligament a dental prosthesis, a muscle implant, or a nerve guide. Complex shapes such as shapes of wounds chambered organs or sleeves that can fit over organs or other structures can be formed. The shapes of the sealant matrices in some embodiments induce cells seeded into the matrices to differentiate in a specific manner. Growth factors or other substances may be incorporated as discussed elsewhere herein. This can result in a more effective, more natural-like organ or tissue being created. Hollow matrices to be filled with desirable substances such as cells or to replace hollow organs or structures are also made. For a cylindrical-shaped sealant composition or any other shape of construct in which an enclosed area is desired, a suture, glue, staple or heat seal or some other method may be used to seal one end of the sealant. This results in a hollow platform that is closed on one end and open on the other. The electroprocessed platform can now be filled with cells or other substances, or cells or other substances may be placed on the outer surface of the construct. For example, a mixture of electroprocessed material, or other substances such as cells, or molecules such as drugs or growth factors may be placed within the platform. The free and open end of the envelope that was used to fill the construct with substances can be sutured, glued or heat sealed shut to produce an enclosed bioengineering platform. Mixing cells with the material during electroprocessing results in cells being distributed throughout the matrix so that they do not have to migrate into the gel. As noted above, however, some electroprocessed materials (such as collagen, for example) have been shown to promote infiltration in some embodiments. The overall three-dimensional geometric shape of the sealant is determined by the ultimate design and type of tissue to be bioengineered. The target in some embodiments is a prosthetic, implant or other object that is to be coated with the electroprocessed material. Examples of coated objects include but are not limited to orthopedic implants or devices (e.g. bone screws, orthopedic spine cages, artificial hip joint components) breast implants, and pacemakers. In some embodiments, the desired shape is determined by medical imaging procedures (e.g. magnetic resonance imaging, computer assisted tomography) and the electroprocessed materials are prepared accordingly. In many embodiments, the electroprocessed structures are seamless. In some other embodiments, the electroprocessed material is incorporated into a woven mesh to be used a sealant or patch (for example, a VICRYL mesh for a hernia patch). In some embodiments a sealant is placed over an organ or tissue. For example a sheet or cylinder of electroprocessed fibrinogen and collagen is placed as a sleeve over the end of a muscle and extends along the tendon. Optionally, thrombin is added to the sleeve. This type of construct is used, for example, to reinforce the muscle tendon attachment or the tendon bone attachment or to reconstruct a severed tendon. In some embodiments the conversion of fibrinogen to fibrin before, during, or after electroprocessing increases the density and/or reduces the porosity of other electroprocessed materials, providing another means to manipulate the strength and other material properties of the resulting matrix.

Shapes of electroprocessed materials can also be controlled by electroprocessing parameters. Powders or droplets that dry to form powders are made by controlling electroprocessing parameters. Powders or particles are also formed by encapsulating substances and electroprocessing encapsulated particles.

Control of shape is also accomplished by manual processing of the formed sealant matrices. For example, formed matrices can be sutured, sealed, stapled, or otherwise attached to one another to form a desired shape. Alternatively, the physical flexibility of many matrices allows them to be manually shaped to a desired structure. In some embodiments, powders are prepared from electroprocessed materials that are pulverized into a powder, sometimes after freezing. In some embodiments electroprocessed materials are wound or woven into threads or sutures, converted into a fluff or batt, woven into fabrics, combined with other substances (such as polyethylene glycol) to form a paste, or pressed or formed into orthopedic inserts or implants. In some embodiments, sutures and large diameter fibers are incorporated into an electroprocessed structure to facilitate placement. The foregoing are only examples and any type of shaping and any shape of electroprocessed material, whether during or after electroprocessing, is within the present invention.

Where mats or sheets are used, structures of different shapes and sizes can be prepared and packaged in desired sizes. Alternately, sheets and mats can be packaged in sizes that can be readily torn or cut into desired shapes. Examples of preferred sizes and shapes include, but are not limited to: 3 cm diameter circles, 5×5 cm squares, and 5×10 cm rectangles. Sheets and mats can have any thickness, with embodiments ranging from tens of nm up to millimeters in thickness. The preferred thickness will vary depending on factors such as, for example, the desirability that the sheet be more flexible (generally favoring a thinner mat) or capable of sealing high flow wounds (generally favoring a thicker mat). In one embodiment, thickness ranges between about 0.05 and about 5.0 mm. In another embodiment, thickness ranges between about 0.2 and about 0.8 mm. In another embodiment, thickness is about 0.5 mm.

In some embodiments, constructs are made of two or more separate electroprocessed structures. A variety of shapes is therefore possible. In one embodiment, a sheet electroprocessed from one or more solutions of collagen and PGA is prepared. Fibrinogen and prothrombin or thrombin is then applied to the sheet. A second sheet comprising a blend of electroprocessed material is then added to the first, forming a sandwich structure with the fibrinogen in the middle layer. Optionally, the edges of the “sandwich” structure are sealed (for example, by heat sealing) to enclose the fibrinogen layer.

### Methods of Making the Electroprocessed Compositions

### Electroprocessing

The methods of making the electroprocessed compositions used in the sealants of the present invention include, but are not limited to electroprocessing structural sealant materials (for example, electroprocessed collagen, fibrinogen, thrombin, fibronectin, or combinations thereof) and optionally electroprocessing other materials, substances or both. As defined above, one or more electroprocessing techniques, such as electrospin, electrospray, electroaerosol, electrosputter, or any combination thereof, may be employed to make the electroprocessed matrices in the compositions of the present invention. In the most fundamental sense, the electroprocessing apparatus for electroprocessing material includes an electroprocessing mechanism and a target substrate. The electroprocessing mechanism includes a reservoir or reservoirs to hold the one or more solutions, melts, or other materials that are to be electroprocessed. The reservoir or reservoirs have at least one orifice or nozzle to allow the streaming of the solution from the reservoirs. Although the terms “orifice” and “nozzle” are used throughout, these term are not intended to be limiting, and refer generically to any location from which solutions may stream during electroprocessing. One or a plurality of nozzles may be configured in an electroprocessing apparatus. If there are multiple nozzles, each nozzle is attached to one or more reservoirs containing the same or different solutions or other materials. Similarly, there can be a single nozzle that is connected to multiple reservoirs containing the same or different materials. Multiple nozzles may be connected to a single reservoir or to different reservoirs. Because different embodiments involve single or multiple nozzles and/or reservoirs, any references herein to one or nozzles or reservoirs should be considered as referring to embodiments involving single nozzles, reservoirs, and related equipment as well as embodiments involving plural nozzles, reservoirs, and related equipment. The size of the nozzles can be varied to provide for increased or decreased flow of solutions out of the nozzles. One or more pumps used in connection with the reservoirs can be used to control the flow of solution streaming from the reservoir through the nozzle or nozzles. The pump can be programmed to increase or decrease the flow at different points during electroprocessing. In this invention pumps are not necessary but provide a useful method to control the rate at which material is delivered to the electric field for processing. Material can be actively delivered to the electric field as a preformed aerosol using devices such as air brushes, thereby increasing the rate of electroprocessing and providing novel combinations of electroprocessed materials. Nozzles may be programmed to deliver electroprocessed material simultaneously or in sequence.

The electroprocessing occurs due to the presence of a charge in either the orifices or the target, while the other is grounded. In some embodiments, the nozzle or orifice is charged and the target is shown to be grounded. Those of skill in the electroprocessing arts will recognize that the nozzle and solution can be grounded and the target can be electrically charged. The creation of the electrical field and the effect of the electrical field on the electroprocessed materials or substances that will form the electroprocessed composition occur whether the charge is found in the solution or in the grounded target. In different embodiments, the space between the target and the nozzle or source of the materials can contain air or selected gases. In various embodiments, the space can be maintained under a vacuum or below atmospheric pressure or above normal atmospheric pressure. Solvents used in electroprocessing typically evaporate during the process. This is considered advantageous because it assures that the electroprocessed materials are dry. In embodiments using water or other less volatile solvents, electroprocessing may optionally occur in a vacuum or other controlled atmosphere (for example, an atmosphere containing ammonia) to assist evaporation of the solvent or the condensation of the electroprocessed material. Humidity of the environment is controlled in some embodiments, to result in any desired humidity from 0% to 100%. Use of vacuum or controlled environment is not limited to such embodiments. Flow of gas in the electroprocessing chamber is also manipulated, for example by causing laminar or non-laminar air or gas flow in a particular direction. Electroprocessing can be oriented varying ways with respect to gravity forces or occur in a zero gravity environment. The temperature of the ambient air and any liquid from which the material is electroprocessed can also be manipulated. In some embodiments, the temperature of a liquid is raised to allow a material to dissolve or become suspended when it would not do so at room temperature.

The substrate can also be used as a variable feature in the electroprocessing of materials used to make the electroprocessed composition. Specifically, the target can be the actual substrate upon which the electroprocessed matrix itself is deposited. Alternatively, a substrate can be disposed between the target and the nozzles. For instance, a petri dish or a conveyor belt can be disposed between nozzles and a target, and a matrix can be formed in the dish or on the belt. Other variations include but are not limited to non-stick surfaces between the nozzles and target. In one preferred embodiment, locations of wounds, tissues or surgical fields (especially areas in which hemostasis or tissue sealing is desired) is disposed between the target and nozzles or is grounded or charged to serve as a target. The target can also be specifically charged or grounded along a preselected pattern so that the solution streamed from the orifice is directed in specific directions. Additional electric fields can be applied to the area of electroprocessing to provide further control of patterns. The electric field can be controlled by a microprocessor to create an electroprocessed matrix having a desired geometry. The target and the nozzle or nozzles can be engineered to be movable with respect to each other, thereby allowing additional control over the geometry of the electroprocessed matrix to be formed. The entire process can be controlled by a microprocessor that is programmed with specific parameters that produce a specific preselected electroprocessed matrix. It is to be understood that any electroprocessing technique may be used, alone or in combination with another electroprocessing technique, to make the compositions of the present invention.

Forms of electroprocessed proteins include but are not limited to preprocessed proteins in a liquid suspension or solution, gelatin, particulate suspension, or hydrated gel or preformed gel. Gels can be electroprocessed by subjecting them to pressure, for example by using a syringe or airbrush apparatus with a pressure head behind it to extrude the gel into the electrical field. In many embodiments, when producing fibers using electroprocessing techniques, especially electrospinning, it is preferable to use the monomer of the polymer fiber to be formed. In some embodiments it is desirable to use monomers to produce finer filaments. In other embodiments, it is desirable to include partial fibers to add material strength to the matrix and to provide additional sites for incorporating substances. Electroprocessed matrix materials such as collagen in a gelatin form may be used to improve the ability of the electroprocessed material to dissolve. Acid extraction method can be used in preparing such gels to maintain the structure of the monomeric subunits. Units can then be treated with enzymes to alter the structure of the monomers.

In embodiments in which two materials combine to form a third material, the solutions containing these components can be mixed together immediately before they are streamed from an orifice in the electroprocessing procedure. In this way, the third material forms literally as the microfibers, particles, powder, or microdroplets are formed in the electroprocessing process. Alternatively, such matrices can be formed by electroprocessing a molecule that can form electroprocessed materials into a moist or otherwise controlled atmosphere of other molecules necessary to allow formation of the matrix to form filaments within the electric field.

Alternatively, in embodiments in which two or more materials each contain different molecules, and those different molecules have the ability to combine or to interact, the two or more materials can be electroprocessed in conjunction with or separately from each other. In some desirable embodiments, this occurs under conditions that do not allow the different molecules in the two or more materials to interact until a desired time. This can be accomplished several ways. For example, in some embodiments the electroprocessed materials are sufficiently dry to prevent interaction. In some embodiments, molecules are encapsulated or mixed with a carrier, such as electroprocessed PEO, polyethylene glycol (PEG), collagen, fibrinogen, fibronectin, fibrin, or other synthetic or natural polymers. The carrier acts to hold the reactants in place until they are initiated. In one preferred embodiment, fibrinogen is electroprocessed and the resulting electroprocessed material is combined with encapsulated thrombin that will release the thrombin pursuant to a desired profile.

It is to be understood that carriers can be used in conjunction with matrix materials. Different materials, such as extracellular matrix proteins, and or substances, can be combined in solutions for electroprocessing with PEG, PLA, PGA, or other known carriers that form filaments. For example, proteins (such as collagen, fibrinogen, or combinations thereof) can be electrospun from solutions containing PEG, PLA, PGA or other known carriers that form filaments. This produces “hairy filaments” with the filaments being PEG, PLA, PGA or other carriers and the “hair” being the protein. The “hairs” cross-link the surrounding matrix carrier into a gel, or provide reactive sites for cells to interact with the substance within the matrix carrier, such as immunoglobulins. This approach can be used for forming a matrix or gelling molecules that do not normally gel.

Alternatively, the electroprocessed material can be sputtered to form a sheet. Examples of molecules that form these sheets include PGA, PLA, a copolymer of PGA and PLA, collagen, and fibronectin. In some embodiments, a sheet is formed with two or more electroprocessed materials that can combine to form a third electroprocessed material when in a moist environment, such as in contact with tissue. This sheet can be placed in a wet environment to allow conversion to the third electroprocessed material.

In addition to the multiple equipment variations and modifications that can be made to obtain desired results, similarly the liquids from which the materials are electroprocessed can be varied to obtain different results. For instance, any solvent or liquid in which the material is dissolved, suspended, or otherwise combined without deleterious effect on the process or the safe use of the matrix can be used. Materials or the compounds that form materials can be mixed with other molecules, monomers or polymers to obtained desired results. In some embodiments, polymers are added to modify the viscosity of the solution. In still a further variation, when multiple reservoirs are used, the ingredients in those reservoirs are electroprocessed separately or joined at the nozzle so that the ingredients in the various reservoirs can react with each other simultaneously with the streaming of the solution into the electric field. Also, when multiple reservoirs are used, the different ingredients in different reservoirs can be phased in temporally during the processing period. These ingredients may include substances.

Embodiments involving alterations to the electroprocessed material itself are within the scope of the present invention. Some materials can be directly altered, for example, by altering their carbohydrate profile or the amino acid sequence of a protein, peptide, or polypeptide. Chitin can be electroprocessed or can be converted to chitosan and electroprocessed. Also, other materials can be attached to the materials before, during or after electroprocessing using known techniques such as chemical cross-linking or through specific binding interactions. Further, the temperature and other physical properties of the process can be modified to obtain different results. The matrix may be compressed or stretched to produce novel material properties.

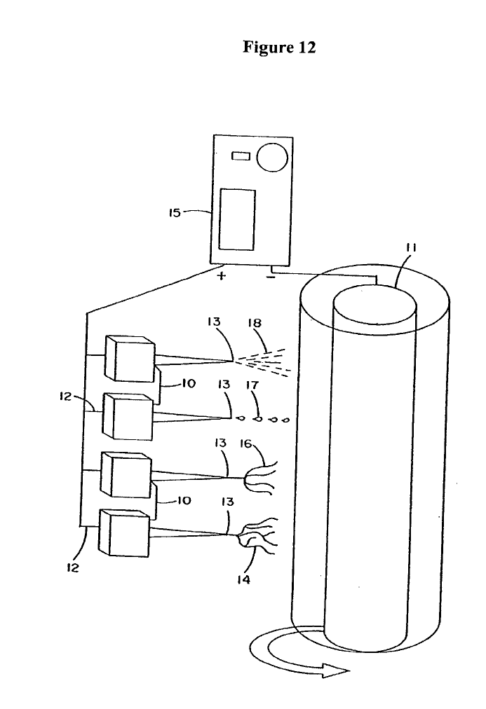

Various effective conditions can be used to electroprocess a matrix. While the following is a description of a preferred method, other protocols can be followed to achieve the same result. Referring to FIG. 11, in electrospinning fibers, micropipettes 10 are filled with a solution comprising the material (for example, collagen, fibrinogen, fibronectin, or combinations thereof) and suspended above a grounded target 11, for instance, a metal ground screen placed inside the central cylinder of the RCCS bioreactor. Although this embodiment involves two micropipettes acting as sources of materials, the present invention includes embodiments involving only one source or more than two sources. A fine wire 12 is placed in the solution to charge the solution in each pipette tip 13 to a high voltage. At a specific voltage determined for each solution and apparatus arrangement, the solution suspended in each pipette tip is directed towards the grounded target. This stream 14 of materials may form a continuous filament, for example when the material is electroprocessed from a solution containing collagen or fibrinogen, such that upon reaching the grounded target, the electroprocessed material collects and dries to form a three-dimensional, ultra thin, interconnected matrix of electroprocessed fibers. Depending upon reaction conditions a single continuous filament may be formed and deposited in a non-woven matrix.

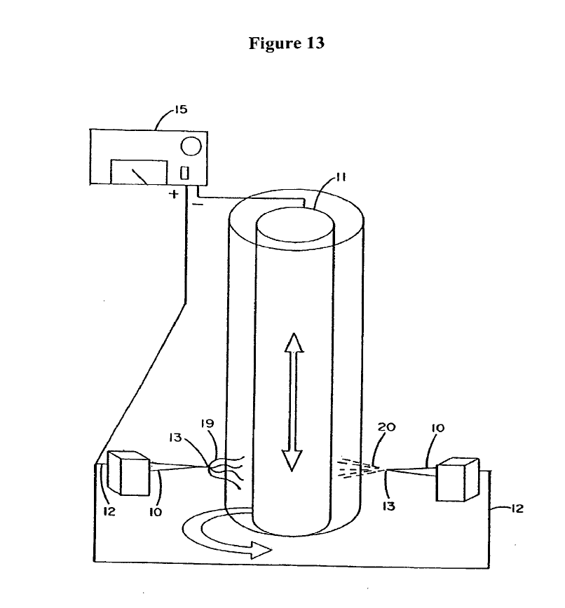

As noted above, combinations of electroprocessing techniques and substances are used in some embodiments. Referring now to FIG. 12 micropipette tips 13 are each connected to micropipettes 10 that contain different materials or substances. The micropipettes are suspended above a grounded target 11. Again, fine wires 12 are used to charge the solutions. One micropipette produces a stream of collagen fibers 14. Another micropipette produces a steam of fibers 16 electrospun from a solution of fibrinogen. A third micropipette produces an electroaerosol of cells 17. A fourth micropipette produces an electrospray of droplets containing thrombin 18.

Similarly, referring now to FIG. 13, electroprocessed material is applied as electrospun fibers 19 from a solution of fibrinogen released by one of the two micropipettes and electrosprayed droplets containing thrombin 20 from the other micropipette disposed at a different angle with respect to the grounded substrate 11. The micropipette tips 13 are attached to micropipettes 10 that contain varying concentrations of materials and thus produce different types of electroprocessed streams despite using the same voltage supply 15 through fine wires 12.

Minimal electrical current is involved in this process, and, therefore, electroprocessing does not denature the materials that are electroprocessed, because the current causes little or no temperature increase in the solutions during the procedure. In melt electroprocessing, there is some temperature increase associated with the melting of the material. In such embodiments, care is exercised to assure that the materials or substances are not exposed to temperatures that will denature or otherwise damage or injure them.

An electroaerosoling process can be used to produce a dense, mat-like matrix of droplets of electroprocessed material. The electroaerosoling process is a modification of the electrospinning process in that the electroaerosol process utilizes a lower concentration of materials or molecules that form electroprocessed materials during the procedure. Instead of producing a splay of fibers or a single filament at the charge tip of the nozzle, small droplets are formed. These droplets then travel from the tip to the substrate to form a sponge-like matrix composed of fused droplets. In some embodiments, the droplets are less than 10 μm in diameter. In other embodiments a construct composed of fibrils with droplets, like “beads on a string” may be produced. Droplets may range in size from 100 nm to 10 μm depending on the polymer and solvents.

As with the electrospinning process described earlier, the electroaerosol process can be carried out using various effective conditions. The same apparatus that is used in the electrospinning process, for instance as shown in FIG. 13 is utilized in the electroaerosol process. The differences from electrospinning include the concentration and identity of the materials or substances and/or the voltage used to create the stream of droplets.

One of ordinary skill in the art recognizes that changes in the concentration of materials or substances in the solutions requires modification of the specific voltages to obtain the formation and streaming of droplets from the tip of a pipette.

Electroprocessing may also involve spray or deposition of particles, powders or other solids. In some embodiments, sealant compositions are encapsulated to form particles or powder and the particles or powders are applied by an electroprocessing process. Any method for applying particles or powders may be used.

The electroprocessing process can be manipulated to meet the specific requirements for any given application of the electroprocessed compositions made with these methods. In one embodiment, the micropipettes are mounted on a frame that allows movement of the micropipettes (either together or separately) in the x, y and z planes with respect to the grounded substrate. In some embodiments, the micropipettes are mounted around a grounded substrate, for instance a tubular mandrel. In this way, the materials or molecules that form materials streamed from the micropipettes are specifically aimed or patterned. Although the micropipettes can be moved manually, the frame onto which the micropipettes are mounted is preferably controlled by a microprocessor and a motor that allow the pattern of streaming material to be predetermined by a person making a specific matrix. Such microprocessors and motors are known to one of ordinary skill in the art. For instance, matrix fibers, particles, powders, or droplets can be oriented in a specific direction, they can be layered, or they can be programmed to be completely random and not oriented. These properties allow the resulting sealant to be tailored to specific tissues if desired.

In the electrospinning process, the stream or streams can branch out to form fibers. The degree of branching can be varied by many factors including, but not limited to, voltage, ground geometry, the identity of the polymer, the degree of dryness in the polymer when it deposits on the target, distance from micropipette tip to the substrate, diameter of micropipette tip, and concentration of materials or compounds that will form the electroprocessed materials. Not all reaction conditions and polymers may produce a true multifilament, under some conditions a single continuous filament is produced. Materials and various combinations can also be delivered to the electric field of the system by injecting the materials into the field from a device that will cause them to aerosol. This process can be varied by many factors including, but not limited to, voltage (for example ranging from about 0 to 30,000 volts), concentration of the materials to be electroprocessed in the solvent (for example between approximately 0.010 g/ml and approximately 0.200 g/ml), distance from micropipette tip to the substrate (for example from 0-40 cm), the relative position of the micropipette tip and target (i.e. above, below, aside etc.), and the diameter of micropipette tip (approximately 0-2 mm). Several of these variables are well-known to those of skill in the art of electrospinning microfiber textile fabrics. In some embodiments, increasing the distance from the target increases the pore size in the resulting matrix.

The geometry of the grounded target can be modified to produce a desired matrix. By varying the ground geometry, for instance having a planar or linear or multiple points ground, the direction of the streaming materials can be varied and customized to a particular application. For instance, a grounded target comprising a series of parallel lines can be used to orient electrospun materials in a specific direction. The grounded target can be a cylindrical mandrel whereby a tubular matrix is formed. Most preferably, the ground is a variable surface that can be controlled by a microprocessor that dictates a specific ground geometry that is programmed into it. Alternatively, for instance, the ground can be mounted on a frame that moves in the x, y, and z planes with respect to a stationary micropipette tip streaming material.

The substrate onto which the electroprocessed materials are streamed, sprayed or sputtered can be the grounded target itself or it can be placed between the micropipette tip and the grounded target. The substrate can be specifically shaped as discussed above. Electroprocessing allows great flexibility and allows for customizing the construct to virtually any shape needed. Many matrices are sufficiently flexible to allow them to be formed to virtually any shape. In shaping matrices, portions of the matrix may be sealed to one another by, for example, heat sealing, chemical sealing, and application of mechanical pressure or a combination thereof. An example of heat sealing is the use of crosslinking techniques discussed herein to form crosslinking between two portions of the matrix. Sealing may also be used to close an opening in a shaped matrix. Suturing may also be used to attach portions of matrices to one another or to close an opening in a matrix. It has been observed that inclusion of synthetic polymers enhances the ability of matrices to be heat sealed.

In some embodiments, sealant matrices are electroprocessed onto a target or substrate that moves the formed matrix out of the electroprocessing process as it is formed. An example is a moving conveyor or belt that moves formed strips or sheets of electroprocessed materials. The speeds of the belt and of the formation of the matrix are coordinated such that a continuous sheet, ribbon, or other structure forms and is conveyed by the belt. These parameters are controlled such that the structure has homogeneous composition and dimensions (e.g. depth and width) or has heterogeneous compositions or dimensions. Where variation exists, the variation is characterized by a pattern or by a random variation. Such embodiments may be combined with any materials handling method used in textile, paper, or other industries as part of produce formation and processing. Optionally, the process uses air flow, negative or reduced air pressure (suction), electrostatic fields, or electromagnetic fields to alter the direction of movement of electroprocessed materials. Such procedures are used, for example, to bring together or to entangle fibers or other electroprocessed materials produced by multiple nozzles. Jets of air or needles can be used to process and to entangle fibers and other matrix structures. In some embodiments, the resulting sheets or other continuous outputs are then manipulated, for example, by bending, heat sealing, welding, crimping, or any other processing desired. Procedures and methods used in the textile, paper, or other industries industry to processes fabrics, webs, and other constructs or structures are examples of processes that can be used.

The material to be electroprocessed can be present in the solution at any concentration that will allow electroprocessing. In one desirable embodiment, the materials to be electroprocessed are present in the solution at concentrations between 0 and about 1.000 g/ml. In another desirable embodiment, the materials to be electroprocessed are present in the solution at concentrations between 0 and about 0.100 g/ml. In another desirable embodiment, the materials to be electroprocessed are present in the solution at concentrations between 0 and about 0.085 g/ml. In another desirable embodiment, the materials to be electroprocessed are present in the solution at concentrations between 0 and about 0.045 g/ml. In another desirable embodiment, the materials to be electroprocessed are present in the solution at concentrations between 0 and about 0.025 g/ml. In another desirable embodiment, the materials to be electroprocessed are present in the solution at concentrations between 0 and about 0.005 g/ml. Examples of desirable embodiments also include, without limitation, those in which the materials to be electroprocessed are present in the solution at concentrations in each of the following ranges: between approximately 0.025 g/ml and approximately 0.045 g/ml; between approximately 0.045 g/ml and approximately 0.085 g/ml; between approximately 0.085 g/ml and approximately 0.100 g/ml; and between approximately 0.100 g/ml; and approximately 1.000 g/ml. Some specific examples of desirable embodiments include: Type I collagen electrospun from a concentration of approximately 0.083 g/ml in 1,1,1,3,3,3 hexafluoro-2-isopropanol (HFP); Type III collagen electrospun from a concentration of approximately 0.04 g/ml in HFP; Type I collagen at a concentration of about 0.0393 g/ml in HFP; a solution containing about 0.1155 grams collagen and about 0.1234 grams of elastin from ligamentum nuchae in 5 ml HFP; Type II collagen at a concentration of about 0.080 to about 0.100 g/ml in HFP; Type II collagen at a concentration of about 0.04 g/ml in HFP; type I collagen at a concentration of about 0.100 g/ml in 2,2,2-trifluoroethanol; elastin electrospun from a solution of about 70% isopropanol and about 30% water containing about 250 mg/ml of elastin; A blend of Type I and Type III collagens at a total concentration of about 0.06 g/ml (Type I at about 0.08 g/ml and Type III at about 0.04 g/ml) in HFP; blends of elastin and numerous collagen types at a total concentration of about 0.075 g/ml; and about 5 mg/ml collagen from an aqueous solution electroprocessed in a vacuum chamber.

Any relative concentration of materials may be used. Some examples include, but are not limited to: embodiments in which the material contains substantially pure Type I collagen; embodiments in which the material contains substantially pure product fibrinogen; embodiments in which the material contains about 58% fibrinogen and about 42% Type I collagen; embodiments in which the material contains another type of collagen (e.g. Type II collagen, Type III collagen, etc. in a substantially pure amount), embodiments containing more than one type of collagen in varying amounts (e.g. an electrospun blend of Type I and Type III collagen, a blend of Type I and Type II collagen, etc.); and embodiments containing one or more type of collagen along with other natural or synthetic materials or both (e.g. blend of about 45% Type I collagen/about 35% Type III collagen/about 20% elastin, blends of about 80% Type I collagen and about 20% elastin, a blend of about 80% Type I collagen/about 10% PGA/about 10% PLA, a blend of about 80% Type I collagen and about 20% of a PGA:PLA copolymer, etc.)

Other variations of electroprocessing, particularly electrospinning and electroaerosoling include, but are not limited to the following:

1. Using different solutions to produce two or more different fibers, particles, powders, droplets, or combinations simultaneously (arrays of fibers, particles, powders, droplets, or combinations). In this case, the single component solutions can be maintained in separate reservoirs. Single or multiple charge sources can be used to generate the potential necessary to induce electroprocessing.

2. Using mixed solutions (for example, materials along with substances such as cells, growth factors, or both) in the same reservoir(s) to produce fibers, powders, particles or droplets composed of electroprocessed materials as well as one or more substances (fiber composition “blends”). Nonbiological but biologically compatible material can be mixed with a biological molecule.

3. Utilizing multiple potentials applied for the different solutions or the same solutions.

4. Providing two or more geometrically different grounded targets (i.e. small and large mesh screens).

5. Placing the mold or mandrel or other ungrounded target in front of the grounded target.

6. Applying agents such as Teflon onto the target to facilitate the removal of electroprocessed materials from the target (i.e. make the electroprocessed material more slippery so that the electroprocessed materials do not stick to the target).

7. Forming an electroprocessed material that includes components applied using multiple electroprocessing methods. For example, electrospun fibers, electroprocessed powders or particles, and electroaerosol droplets in the same composition can be beneficial for some applications depending on the particular structure desired. This combination of structures can be obtained by using the same micropipette and solution and varying the electrical charge; varying the distance from the grounded substrate; varying the polymer concentration in the reservoir; using multiple micropipettes or sources of electroprocessed materials, (e.g. some for streaming fibers and others for streaming droplets); or any other variations to the method envisioned by those of skill in this art. The fibers, powders, particles, and droplets can be layered or mixed together in same layers. In applications involving multiple micropipettes, the micropipettes can be disposed in the same or different directions and distances with reference to the target.

8. Using multiple targets.

9. Rotating targets or mandrels during electroprocessing to cause the electroprocessed materials to have a specific polarity or alignment, using vibrating targets, or causing oscillatory movement of targets.

10. Multiple materials or different concentrations of materials prepared in separate reservoirs that are combined at or prior to entry into the electrical field (e.g. multiple reservoirs that are channeled together to a single nozzle or syringe). By controlling the relative rate and volume at which a concentrated solution and a less concentrated solution are delivered to the mixing point a gradient of electroprocessed material can be produced (e.g. 5% to 17%). A similar arrangement for electrospinning can be used to make a composition composed of a continuum of different fiber diameters.

All these variations can be done separately or in combination to produce a wide variety of electroprocessed materials and substances.

The various properties of the electroprocessed materials can be adjusted in accordance with the needs and specifications of the cells to be suspended and grown within them. The porosity, for instance, can be varied in accordance with the method of making the electroprocessed materials or matrix. Electroprocessing a particular matrix, for instance, can be varied by size and density of fibers, particles, powder, or droplets. If the cells to be grown in the matrix require a great deal of nutrient flow and waste expulsion, then a loose matrix can be created. On the other hand, if the tissue to be made requires a very dense environment, then a dense matrix can be designed. Porosity can be manipulated, for example by regulating rates or degrees of hydration of the resulting matrix, extent and type of cross-linking or by mixing salts or other extractable agents. Removing the salt will leave holes of defined sizes in the matrix.

In one embodiment for electroprocessing collagen, the appropriate approximate ranges are: voltage 0-30,000 volts; pH 7.0 to 8.0; temperature about 20 to about 42° C.; and collagen 0 to about 5 mg/ml. One embodiment for electrospraying collagen uses collagen at a concentration of about 0.080 g/1.0 ml acid extracts of Type I collagen (calfskin) dissolved in HFP, electroprocessed from a syringe at 25 kV at a distance from the target of about 127 mm and a syringe pump rate of 10 ml/hr. At this concentration the collagen did not exhibit any evidence of electrospinning (fiber formation) and, regardless of the input voltage, the polymer solution formed electrosprayed droplets and leakage from the syringe tip. One embodiment for elastin uses elastin from ligamentum nuchae dissolved in 70% isopropanol/water at a concentration of 250 mg/ml. The solution is then agitated to ensure mixing and loaded into a 1 ml syringe. Once loaded, the syringe is placed onto a syringe pump and set at a flow rate of 10 ml/hr. A mandrel is placed 7 inches from the syringe tip and rotated at a selected speed. The pump and power supply are then turned on and the voltage is set for 24,000 kV. Electroprocessed materials of varying properties can be engineered by shifting the pH, changing the ionic strength (e.g. addition of organic salts), or adding additional polymeric substrates or cationic agents. For example, increasing the number of protons (making solvent more acidic) may be expected to disrupt hydrogen bonding between adjacent hydrogens in a peptide. Similarly, reducing the number of hydrogen protons (making solvent more basic) present in the solvent may be used to promote hydrogen binding between adjacent amino acids in a protein peptide. Selecting an electrospinning solution with a moderately acidic or moderately alkaline pH would thus be expected to enhance the tendency to form electrospun fibers rather than an electrosprayed material. Embodiments for electrospinning fibrinogen or blends of fibrinogen and collagen may be found in the Examples herein. In some embodiments, increasing ionic concentration, especially of bivalent cations, provides a means of control by which fiber diameter and pore size are decreased.

Methods of Combining Substances with Electroprocessed Materials

Substances can be combined with the electroprocessed materials by any of means in the preparation of the sealants. Examples include, but are not limited to, dripping, spraying, brushing, or electroprocessing the substances onto the electroprocessed materials, and immersing the electroprocessed materials into the substances. In some embodiments, substances are combined with electroprocessed materials during the formation of the sealant. One embodiment involves spraying, atomizing, dripping, dribbling, or otherwise placing the substance into the space between the nozzles from which the solutions are electrospun and the target or substrate such that the substance is trapped or entangled by the electroprocessed material as the electroprocessed material crosses the air gap between the source solutions and target. One embodiment involves placing or applying the substance onto the target or mandrel as the material is electroprocessed. In some embodiments, the substance comprises molecules to be released from or contained within the electroprocessed material and is therefore added to or incorporated within the matrix of electroprocessed material. Substances can be mixed in the solvent carriers or solutions of materials for electroprocessing. In this system materials can be mixed with various substances and directly electroprocessed. The resulting composition comprising an electroprocessed matrix and substance can be topically applied to a specific site and the substances released from the electroprocessed material as a function of the electroprocessed material undergoing breakdown in the surrounding environment. Substances may also be released from the electroprocessed compositions of the present invention through diffusion.

The state of the electroprocessed material in relation to the incorporated substances is dictated and can be controlled by the chemistry of the system and varies based on the selection of electroprocessed materials, solvent(s) used, and solubility of the materials in those solvents. These parameters can be manipulated to control the release of the substances (or other elements) into the surrounding environment. If substances to be incorporated into the electroprocessed material are not miscible with the material, separate solvent reservoirs for the different components can be used. Thus, substances that are not miscible with material to be electroprocessed can be mixed into solvent carriers for other materials to be electroprocessed along with the material from, for example, separate reservoirs. Mixing in such an embodiment occurs prior to, during, and/or after deposition on the target, or a combination thereof. It is to be understood that substances may be entrapped or entangled within an electroprocessed material, bonded to a material before the material undergoes electroprocessing, or bound to specific sites within the electroprocessed material.

In some embodiments, immiscible molecules can be electroprocessed from a single reservoir through the preparation of a two-phase suspension in which one molecule is contained in particles or droplets suspending in a fluid containing the other molecule. For example, in one embodiment a solution containing a substance that is immiscible with the material to be electroprocessed is suspended within another solution containing the material to be electroprocessed, and directly electrospun together. In one embodiment, a chemical agent such as surfactant is used to create an emulsion or dispersion of one phase within the other. Examples of surfactants that can be used include, for example, any ionic or non-ionic surfactants. Specific examples include, but are not limited to, lung surfactant, bovine serum albumin, fatty acid salts (e.g., sodium lauryl sulfate), Tween, and non-ionic substances such as Tritons (oligoethylene oxide-modified phenols) or Pluronics (ethylene oxide-propylene oxide-ethylene oxide block copolymers). Any means to impart energy sufficient to create an emulsion or to disperse one phase in another may also be used. Physical means such as ultrasonic homogenization or other techniques of physical agitation, homogenization, or blending may be used. One example of known homogenization techniques are those used to induce a uniform distribution of lipid droplets within whole milk products. In one embodiment, hydrophobic proteins have been suspended in droplets within an aqueous solution containing poly(ethylene-co-vinyl acetate) (EVA). Electrospinning this liquid resulted in EVA fibers containing the protein. In another embodiment, yeast cells have been similarly suspended to result in individual EVA fibers containing yeast cells. In some embodiments, the material to be electroprocessed is manipulated to alter its solubility in a given solvent. For example, residues are added or removed to alter the charge of a material and thereby to make the material more or less soluble in a given solvent system. In one embodiment, sugar residues are added or removed from a protein.

In some embodiments, the substance is a particle or aggregate comprising a matrix of compounds or polymers such as alginate that, in turn, contain one or more compounds that will be released from the electroprocessed material. Substances such as drugs or cells can be combined with alginate by, for example, combining a drug suspension or drug particulate in the alginate in the presence of calcium. In one preferred embodiments, particles or aggregates containing thrombin are combined with electroprocessed fibrinogen. Alginate is a carbohydrate that forms aggregates when exposed to calcium. The aggregates can be used to trap drugs. The aggregates dissolve over time, releasing the substances trapped in alginate. The particles, which are then incorporated within the larger electroprocessed matrix, are biologically compatible but relatively stable and will degrade gradually. In some embodiments, the electroprocessed materials resemble a string of pearls. This is a physical aspect of the electroprocessing. If the concentration of materials to be electroprocessed is low, electrospraying of beads occurs. As the concentration increases there are some beads and some fibers. A further increase in concentration of materials to be electroprocessed leads to predominantly or all fibers. Therefore, the appearance of the pearls on a string is a transition phase.

If a substance does not bind or interact with an electroprocessed material, the substance can be entrapped for example, in PGA or PLA pellets, or electroaerosoled to produce pellets in the electrospun material. Several drugs (for example, penicillin) can be trapped in this manner. The pellets or electroaerosoled droplets containing the substance begin to dissolve after administration to deliver the entrapped substance. Some agents can be coupled to synthetic, or natural polymers by a covalent bond, prior to or after spinning.

In other embodiments, the substance is electroprocessed. Substances can be electroprocessed from the same orifice as the materials being electroprocessed or from different orifices. Substances can also be subjected to the same or a different type of electroprocessing as the material. A molecule can be bonded to the electroprocessed material directly or through linking to a molecule that has an affinity for the electroprocessed material. An example of this embodiment involves bonding polypeptide substances to heparin, which has an affinity for collagen. This embodiment allows release rates to be controlled by controlling the rate of degradation of the electroprocessed material, for example by enzymatic or hydrolytic breakdown.

In other embodiments, the electroprocessed material can entrap substances during the electroprocessing process. This can be accomplished by disposing substances in the space between the source of the electroprocessed stream and the target for the electroprocessed material. Placing such substances in the space between the source and target can be accomplished by a number of methods, including, but not limited to, suspending in air or other gases, dripping, spraying, or electroprocessing the substances. The substances can be present in that space in, for example, particulate, aerosol, colloidal, or vapor form. In these embodiments, the electroprocessed material or matrix will physically entrap the substances. This embodiment can also be used to encapsulate larger particles, such as cells, large particles, or tablets. For example, if a tablet is dropped through the matrix as it forms, the tablet is surrounded by the matrix. If a small object, is dropped through the matrix as it forms, or is placed in an aerosol within the matrix, the object may be trapped between filaments, within filaments or attached to the outside of the filaments. For example, by suspending objects in a solution or within a matrix, the objects can become part of an electrospun matrix during fabrication of the filaments. Alternatively, encapsulation can occur by dropping substances onto or through electroprocessed material stream as a matrix forms. The objects thus become surrounded by a matrix of electroprocessed material. These embodiments can be used to incorporate within a matrix substances that are not soluble and/or are too large to form a suspension in the solvent used for the production of the electroprocessed material. For substances in a mist or vapor form, controlling distribution and composition of substances in the space between the source and target can be used to alter the physical and chemical properties of the electroprocessed material and the pattern of distribution of the substances in the electroprocessed material. For all of the foregoing embodiments, the substances can be placed in the electroprocessed material in capsules, vesicles, or other containments for subsequent release. Since the solvent carrier often evaporates in the electroprocessing technique as the electroprocessed material forms, such as a filament, substances may be placed in the electroprocessed matrix and solvent toxicity is greatly reduced or eliminated.

In some embodiments the electroprocessed material is treated after electroprocessing in a manner that will cause it to entrap a substance. For a example, some electroprocessed materials are hydrated in the presence of the substance to trap that substance within the matrix upon expansion or swelling of the fibers.

In many embodiments the substance comprises cells. Cells can be combined with an electroprocessed sealant by any of the means noted above for combining small objects in a matrix. Cells can, for example, be suspended in a solution or other liquid that contains the material to be electroprocessed, disposed in the area between the solutions and target, or delivered to a target or substrate from a separate source before, during, or after electroprocessing. Cells can be dripped through the matrix, onto the matrix as it deposits on the target or suspended within an aerosol as a delivery system for the cells to the electroprocessed material. The cells can be delivered in this manner while the matrix is being formed. As an example, cardiac fibroblasts were suspended in phosphate-buffered saline (PBS) at a concentration of approximately one million cells per milliliter. The suspension of cells was placed within a reservoir of a Paasche air brush. To test the efficacy of using this type of device to deliver cells, the cell suspension was initially sprayed onto a 100 mm culture dish. Some of the cells survived, attached to the dish and spread out over the substratum. In a second trial, the culture dish was located further away from the air brush and the experiment was repeated. Cells were observed on the dish. They appeared to be flattened by the impact and were partially spread out over the surface of the substratum. Culture media was added to the dish and the cells were placed into an incubator. After one hour of culture, the cells were inspected again, and many were found to have spread out further over the substratum. These results demonstrate that a simple airbrush device can be used to place cells into an aerosol droplet and deliver them on demand to a surface or site of interest. Cell viability can be improved by restricting this technique to cells that are resistant to the shear forces produced in the technique, developing a cell suspension with additives that cushions the cells or refining the aerosolizing device to produce a more laminar flow. In addition, directing the cell aerosol into electroprocessed materials as the matrix is forming in the space between the target or mandrel and the source(s) of molecules being electroprocessed produces the effect of cushioning the cells. While not wanting to be bound by the following statement, it is believed that the cells will be trapped in the storm of filaments or other bodies produced by electrospinning or electroprocessing and pulled onto the mandrel. This situation may be less traumatic to the cells than directly spraying the cells onto a solid surface.

In some embodiments, the cells are added either before or at the same time as the materials that are electroprocessed are brought together. In this way, the cells are suspended throughout the three-dimensional matrix formed by electroprocessing.

Cells can be added as the filaments are produced in the space between the target and polymer source. This is accomplished by dripping the cells onto the target, dripping the cells into the electroprocessed matrix as it forms, aerosoling the cells into the matrix or onto the target or electrospraying the cells into the matrix as it condenses and forms near or on the grounded target. In another embodiment, cells are sprayed or dribbled into a forming electroprocessed material or matrix, and are thereby trapped as the electroprocessed material crosses the air gap between the source solutions and target.

An alternative method to deliver cells to electroprocessed material in the formation of sealants involves electroaerosol delivery of the cells. Cells can be deposited by electrostatic spraying at, for example, 8 kV directly onto standard polystyrene culture dishes, suggesting that electrostatic cell spraying is a viable approach. Cardiac fibroblasts in phosphate buffered saline (PBS) have been electroaerosoled up to a 20 kV potential difference. In another example, Schwann cells (rat) were plated on a PS petri dish by conventional methods after one day. Schwann cells were also electrosprayed onto a PS petri dish with a metal ground plate behind the dish at 10 kV after one day. Both samples grew to almost confluence after one week. The electroaerosol approach provides some distinct advantages. First, the shear forces produced during the delivery phase (i.e. the production of the aerosol) appear to be much less traumatic to the cells. Second, the direction of the aerosol can be controlled with a high degree of fidelity. In essence the cell aerosol can be painted onto the surface of interest. This allows the cell to be targeted to specific sites. In electroaerosol delivery, cells are suspended in an appropriate media (e.g. culture media, physiological salts, etc.) and charged to a voltage, and directed towards a grounded target. This process is very similar to that used in electroprocessing, particularly electrospinning. The produces a fine mist of cells trapped within the droplets as they are produced and directed at the grounded target.

Cells can be delivered using aerosol and electroaerosol techniques onto electroprocessed material. The electroaerosol of cells can be delivered in parallel (i.e. alongside) the electroprocessing material or from a separate site. The cells can be delivered to the storm of filaments or particles produced within the air gap in the electroprocessing process or directed at the target. The cells and electroprocessed material also can be delivered in an alternating sequence to the target, i.e. electroprocess the material, aerosol the cells, electroprocess the material, aerosol the cells. This allows for the discrete layering of the construct in separate layers. Furthermore, a vapor source can be provided that directs water onto the mandrel of target used to collect the cells. Providing this moisture improves cell viability by keeping the cells from dehydrating during processing. Cells can be added to the electroprocessed material at any time or from any orientation in any aerosol strategy. Again the advantage of the process in general is that the electroprocessed material collects in a dried state on the target mandrel. Accordingly, although some solvents used in electroprocessing may be toxic, they are lost from the system before the filaments collect on the target.

Cells can also be trapped within a carrier prior to producing an aerosol. For example, cells can be encapsulated within an electroprocessed material like alginate. The encapsulated cells are physically protected from shear and trauma during processing. Cells delivered in this form to the electroprocessed material will have higher viability when sprayed or electrostatically seeded.

In embodiments in which electroprocessed materials are delivered directly to a desired location, additional cells or substances can then be aerosolized onto or into the wound site.

Magnetically and electrically active materials can be electroprocessed, including, for example, preparing conducting polymer fibers produced by electrospinning. In addition, conducting polymers can be prepared in-situ in the matrix by, for example, incorporation of a monomer (e.g., pyrrole) followed by treatment with polymerization initiator and oxidant (e.g., FeCl₃). Finally, conducting polymers can be grown in the electroprocessed material after electroprocessing by using a matrix-coated conductor as the anode for electrochemical synthesis of, for example, polypyrrole or polyaniline. Materials to be electroprocessed can be added to an aqueous solution of pyrrole or aniline to create a conducting polymer at the anode with the entrapped electroprocessed material-forming compounds, which can then be treated with other compounds to allow formation of the electroprocessed material to occur.

More than one method for combining the substances with electroprocessed materials can be used in a single embodiment or application. Combining methods can be especially useful in embodiments in which the electroprocessed material will release one or more substances, and even more so when the released substances are intended to have complex release kinetics, although such combinations are not limited to those embodiments.

Because of the nature of the electroprocessing process, the solvent in many embodiments evaporates completely or nearly so as part of the process. As a result, many electroprocessed compositions contain little solvent or substantially no solvent. This is especially true with more volatile solvents such as HFP or TFE.

### Patterns of Distribution for Electroprocessed Materials and Substances

Many embodiments of the present invention involve means for manipulating a sealant pattern or distribution of electroprocessed material and/or substances within a sealant. For example, an electroprocessing target can also be specifically charged or grounded along a preselected pattern so that electroprocessed materials streamed toward the target are directed into specific directions or distributions on the target or on a substrate. The electric field can be controlled by a microprocessor to create a matrix having a desired geometry. The target and the electroprocessing nozzle or nozzles can be movable with respect to each other and to the target thereby allowing additional control over the geometry of the electroprocessed material to be formed. In embodiments in which substances are electroprocessed, this manipulation will also allow control of the distribution of substances within the electroprocessed materials. For example, an electroprocessed matrix can be prepared on a mandrel, and substances from a separate reservoir can be sprayed, dripped, or electroprocessed in a specific pattern over the existing matrix. In some embodiments, means other than electroprocessing are used to apply substances to electroprocessed materials, (including but not limited to spraying, brush application, airbrush application and dipping), are configured in a way to allow patterned application of substances. This may also be accomplished by simultaneously electrospraying a matrix from one source and a substance from another source. In this example, the matrix source may be stationary and the substance source is moved with respect to the target mandrel. In some embodiments, means other than electroprocessing are used to apply substances to electroprocessed materials, (including but not limited to spraying, brush application, airbrush application and dipping), are configured in a way to allow patterned application of substances to an electroprocessed material. Combinations of methods of controlling the patterns allow creation of complex patterns within the electroprocessed materials.

Other features that allow establishment of such a pattern include, but are not limited to, the ability to deposit multiple layers of the same or different electroprocessed materials, combining different electroprocessing methods, the use multiple orifices with different contents for electroprocessing, and the existence of numerous methods for combining substances with the electroprocessed materials. For example, embodiments exist in which a gradient of substances is created along an electroprocessed material or in which distinct and discrete layers are formed. In some embodiments, layer allows closer mimicry of native tissue. In some embodiments, layering allows creation of electroprocessed materials in which different parts have different properties. In some embodiments, a layer of an electroprocessed material that tends to act as an adhesive is placed between two layers that would otherwise tend to delaminate. Electroprocessed fibrinogen is useful for this purpose in some embodiments.

In embodiments in which the matrix is shaped into a cylindrical construct, for example, the gradient can be prepared along the long axis of a construct (left to right) or the perpendicular axis (inside to out). This configuration is used to provide a chemoattractant gradient to guide the movement of cells within a specified site. Thus, for example, in some embodiments in which neovascular agents are prepared in a perpendicular gradient along an electroprocessed construct, the agents can be concentrated on the dorsal surface of a sheet of the electroprocessed material. The ventral side can be placed against a wound and the higher concentration of angiogenic substances on the dorsal surface of the construct will increase the migration of endothelial cells through the electrospun material. Again, embodiments with complex patterns can use a microprocessor programmed with the specific parameters to obtain a specific, preselected electroprocessed pattern of one or more electroprocessed polymers, optionally with one or more substances.

Embodiments also exist in which electroprocessed materials that degrade or dissolve at a different rate are applied in a given pattern. In some embodiments, substances that accelerate or decrease the rate of degradation of an electroprocessed material are applied in a pattern. For example, in some embodiments using an electroprocessed fibrinogen matrix, patterns of fibrinolytic agents, fibrinolytic inhibitors, or both are used to vary the rate of degradation of the matrix. The result is that the varying rates of degradation causes the formation a change in shape or internal structure of the sealant over time. Such arrangements are used, for example, to create cavities within a matrix (for example, for insertion of cells) or to create pathways for growth of materials such as blood vessels or neurons.

### Additional Processing of Electroprocessed Materials in the Sealants

Electroprocessed materials used in the sealants of the present invention may be further processed to affect various properties. In some embodiments electroprocessed material is cross-linked. In some embodiments, cross-linking will alter, for example, the rate at which the electroprocessed material degrades or the rate at which a substance contained in an electroprocessed matrix is released from the electroprocessed material by increasing structural rigidity and delaying subsequent dissolution of the electroprocessed material.

One preferred crosslinking agent for electroprocessed proteins is glutaraldehyde. In some embodiments using a Type I collagen/Type III collagen/elastin (45:35:20) matrix, exposing the matrix to glutaraldehyde vapor under appropriate conditions for at least about 10 minutes provided a satisfactory degree of cross-linking. In general, longer intervals of glutaraldehyde cross-linking increase the stability of the matrix, but reduce cellular infiltration. A desirable range is exposure for between about 10 and about 20 minutes. Longer periods of crosslinking are appropriate for embodiments that will be used in environments where trauma and mechanical activity may be more intense, greater crosslinking is desired. Persons of skill in the art will understand that the duration varies depending on the composition of the electroprocessed material and the characteristics and concentration of the crosslinking agent. Exposure was accomplished by preparing a gas chamber made by placing a sterile 10 cm² petri dish with its top removed into the center of a 35 cm² petri dish with its top remaining in place. Approximately 4 ml of the 3% glutaraldehyde solution was placed into the smaller dish and the collagen mats were placed in the in the larger dish toward the edges. The 3% glutaraldehyde solution was made by mixing 50% glutaraldehyde with distilled water and 0.2 M sodium cacodylate buffer.

Crosslinking is one of many factors that allows control over the mechanical properties of an electroprocessed matrix in a sealant. A variety of mechanical properties are possible. Examples include but are not limited to: a dry sample of Type I collagen electrospun fiber scaffold, crosslinked by exposure to glutaraldehyde vapor for approximately 2.5 hours, having an elastic modulus of 52 MPa and a peak stress of 1.5 MPa; a Type I collagen electrospun fiber scaffold, also crosslinked by exposure to glutaraldehyde vapor for approximately 2.5 hours, then hydrated in PBS for three hours, having an elastic modulus of 0.2 MPa with a peak stress of 0.1 MPa; and a Type I collagen electrospun fiber scaffold, crosslinked by exposure to glutaraldehyde vapor for 24 hours, then hydrated in PBS for three hours, having a modulus of 1.5 MPa with a peak stress of 0.25 MPa; uncrosslinked Type II collagen scaffolds revealed a tangent modulus of 172.5 MPa and an ultimate tensile strength of 3.298 MPa. In preferred embodiments, mechanical properties of the electroprocessed matrix are within ranges found within natural extracellular matrix materials and tissues. Examples include, but are not limited to, matrices with a dry elastic modulus between about 0.5 and about 10 MPa and matrices with a dry elastic modulus between about 2 and about 10 MPa. Other examples include, but are not limited to, matrices with a dry peak stress between about 0.5 and about 10 MPa and matrices with a dry peak stress between about 1 and about 5 MPa. These values for elastic modulus and peak stress are not intended to be limiting, and electroprocessed matrices with any type of mechanical properties are within the scope of this invention.

Additional substances can be applied to the electroprocessed material in the sealant after formation, for example by soaking the electroprocessed material in the substance or a solution containing the substance or by spraying the solution or substance onto the electroprocessed material. Matrices placed in contact with cells in vitro or in vivo, will be infiltrated by cells migrating into the matrix. Any in vitro method for seeding matrices with cells can be used. Examples include for example, placement in a bioreactor or use of electrostatic cell seed techniques such as those disclosed in U.S. Pat. Nos. 6,010,573, 5,723,324, and 5,714,359. Electroprocessed matrices may also be sterilized using known sterilization methods. For example the electroprocessed material can be immersed in a 70% alcohol solution. Another preferred sterilization method is the peracetic acid sterilization procedure known for certain tissues.

Physical processing of the formed electroprocessed material and the sealants containing such electroprocessed materials is also possible. The electroprocessed matrix may be milled into a powder or milled and prepared as a hydrated gel composed of banded fibrils. In some embodiments, mechanical forces, such as compression, applied to an electroprocessed material hasten the breakdown of the matrix by altering the crystalline structure of the electroprocessed material. Structure of the matrix is thus another parameter that can be manipulated to affect release kinetics. Polyurethanes and other elastic materials such as poly(ethylene-co-vinyl acetate), silicones, and polydienes (e.g., polyisoprene), polycaprolactone, polyglycolic acid and related polymers are examples of polymers that form electroprocessed materials whose release rate can be altered by mechanical strain.

### Further Processing of Sealants Relating to Tissue Growth

Once an electroprocessed sealant containing electroprocessed material and cells is assembled, the sealant can be inserted into a recipient. Where cells are contained in the sealant, the structure can be placed into a culture to enhance the cell growth. Different types of nutrients and growth factors can be added to a culture (or administered to a recipient) in order to promote a specific type of growth. In one example, specifically in connection with the preparation of an engineered muscle tissue, the sealant containing electroprocessed material and cells can be mechanically or passively strained or electrically preconditioned (stimulating electrically sensitive cells, such as cardiac and skeletal muscle cells to contract by electrical depolarization) in order to stimulate the alignment of cells to form a functional muscle implant. Applying strain also increases the tensile strength of the implant. For example, forceful contraction or stretching of cells will lead to hypertrophy as if they were subjected to stretch. In a skin patch, application of mechanical stress may facilitate orientation of the skin for use in an area such as the scalp that is exposed to significant stretching force. Other sealants that may benefit from the application of strain include, but are not limited to, sealants used in muscle tissues, ligament tissues, and tendon tissues. Passive strain in this context refers to a process in which strain is induced by the cells themselves as they contract and reorganized a matrix. This is typically induced by fixing the ends of the electroprocessed matrix. As the cells contract and alter the matrix the fixed ends of the matrix remain in place and thereby strain the cells as they “pull” against the isometric load. The strain not only aligns the cells, it sends signals to them with respect to growth and development. The construct can also be strained externally, i.e. the construct can be prepared and then stretched to cause mechanical alignment. Stretch is typically applied in gradual fashion over time. In some embodiments, electroprocessed materials are stretched to cause alignment in the matrix before the cells are added to the construct (i.e. form the matrix, stretch the matrix and then add the cells). Any known method for applying mechanical or passive physical strain to tissues may be used.

An additional way to combine electroprocessed sealant matrices with cells for implantation is to prepare constructs, then add cells to the constructs. Cells can be placed in a lumen or space within a construct, or implanted adjacent to the implant to facilitate growth. Alternatively, the sealant can be placed in a bioreactor. There are several kinds of commercially available bioreactors, devices designed to provide a low-shear, high nutrient perfusion environment. Until recently, most of the available bioreactors maintained cells in suspension and delivered nutrients and oxygen by sparging, through the use of impellers, or other means of stirring. These methods produce high shear environments that can damage cells or inhibit the formation of large-scale constructs. The RCCS bioreactor (Synthecon) is a rotating wall bioreactor. It consists of a small inner cylinder, which itself can be used as a substrate for electroprocessing, positioned inside a larger outer cylinder. Although the electrospun or electroaerosol matrix can be fabricated on the inner cylinder, other locations within the bioreactor also can be used for placement of a matrix for seeding. For example in some applications it is desirable to allow the scaffolding to float freely within the chamber. The gap between the inner and outer cylinders serves as the culture vessel space for cells. Culture medium is oxygenated via an external hydrophobic membrane. The low shear environment of the Synthecon RCCS bioreactor promotes cell-cell and cell-extracellular matrix (ECM) interactions without the damage or “washing away” of nutrients that occurs with active stirring or sparging. Typically, the RCCS device is operated at rotation rates of 8 up to 60 rpm, as required to maintain cells in suspension, and at less than 8 rpm (preferably 2-3 rpm) for cultures immobilized along the center shaft of the vessel. The Synthecon bioreactor can be used in a standard tissue culture incubator. These values for spin rates and other parameters can be varied depending on the specific tissue created.

In other applications an electroprocessed sealant construct may be fabricated and placed within the RCCS bioreactor and allowed to undergo continuous free fall, a buoyant environment that fosters the formation of large scale, multi-layered constructs. Cells may be added to the construct prior to its placement within the bioreactor. Alternatively, the bioreactor may be used as a platform to seed cells onto the electrospun matrix. For example, a cylindrical construct can be placed within the bioreactor vessel. Cells may be added to the vessel and allowed to interact with the electrospun construct in free fall. The rate required to maintain the constructs in suspension is dependent upon the size and density of the electroprocessed material present in the construct. Larger constructs (2-4 mm in diameter by 10-12 mm in length may require rates of rotation that approach 15-20 rpm. Larger constructs, for example cartilage, can require even higher rates of rotation.

Electroprocessed sealants are useful in formation of prostheses or for use in connection with prosthesis (e.g., as a coating or an adhesive). One application of the electroprocessed matrices is in the formation of medium and small diameter vascular prostheses or for adhesives used to attach such prostheses to vascular anastomoses. Some preferred electroprocessed materials for this embodiment are electroprocessed collagen and elastin, especially collagen type I and collagen type III. Some examples include, but are not limited to coronary vessels for bypass or graft, femoral artery, popliteal artery, brachial artery, tibial artery, radial artery, arterial bifurcation, or corresponding veins. The electroprocessed material is useful especially when combined with endothelial cells on the inside of the vascular prosthesis, and smooth muscle cells, for example a collagen tube, and also when combined with fibroblasts on the outside of the collagen tube. More complicated shapes including tapered and/or branched vessels can also be constructed. A different shaped mandrel is necessary to wind the large fibers around or to orient the electrospun/electroaerosol polymer.

Combination of electroprocessed fibers, such as larger diameter (e.g., 50 to 200 μm) collagen or other fibers can provide optimal growth conditions for cells. The large diameter fibers form a basic structural matrix that lends mechanical support to the sealant, and the electroprocessed matrix is used as a scaffolding to deliver and/or support the cells. This facilitates cell attachment onto the structural matrix. Large scale fibers can be incorporated into or used with bioengineered organs and tissues to lend additional mechanical strength as needed. For example, large fibers can be placed within an electrospun matrix that is designed as a scaffolding or reinforcement for the fabrication of skeletal muscle, cardiac muscle and other smooth muscle based organ such as the intestine and stomach. In an alternative fabrication strategy, a cylindrical construct is electrospun onto a suitable target, for example a cylindrical mandrel. Other shapes can be used if desirable based upon the shape of the site into which the implant will be placed. Examples of matrices in this embodiment include, but are not limited to, electroprocessed collagen, fibrin, fibrinogen, fibronectin, PGA, PLA, and PGA-PLA blends, poly(caprolactone), copolymers of caprolactone with glycolide and/or lactide, poly(hydroxy butyrate) and copolymers, poly(ester-urethanes) and related materials, poly(1,5-dioxepan-2-one) and copolymers, PEO, PVA or other blends, or combinations of the foregoing. The relative ratio of the different components of this construct is tailored to specific applications (e.g. more electroprocessed fibrin or fibrinogen, less electroprocessed collagen for enhanced vascularization in a skin graft). To fabricate a cylindrical muscle the construct is filled with muscle or stem cells or other cell type and the distal ends of the electrospun constructs are sutured or sealed shut. In some embodiments, cells are mixed with various electroprocessed materials to enhance their distribution within the construct. For example, the cells can be mixed with electroprocessed collagen, fibrinogen, fibrin, or combinations thereof prior to insertion into the construct. The objective of this strategy is to provide additional mechanical support to the construct and provide the cells with a three dimensional matrix within the construct to promote growth. This also helps to maintain the cells in an even distribution within the construct. This method can be used to enhance the alignment of the cells within the construct. This filling substance can be extruded directly into the cylindrical construct, as the filling is extruded, alignment occurs. Mixing endothelial cells with the other cells inserted into the construct (or other cell types) is done to accelerate neovascularization. Another method to accomplish this objective is to electroprocess endothelial cells directly into the electroprocessed matrix that aids in formation of the cylindrical sheath. The alignment of the fibers within the electroprocessed matrix that comprises the construct are optionally controlled by controlling the relative movement of the target and source solution with respect to one another. Other cell types, such as tendon fibroblasts, are optionally electrospun or otherwise seeded into or onto the outer surface of the construct to enhance the formation of the outer connective tissue sheath that forms the construct.

In another example, a sheet of electroprocessed material is prepared, rolled into a cylinder and inserted into another electroprocessed cylinder. The construct is filled with cells as described above, sutured shut and placed in a bioreactor or directly in situ. By aligning the fibrils of the electrospun material in parallel with the long axis of the outer cylinder a scaffolding for the production of a muscle-like, electroprocessed composition is produced. Cells in contact with the fibrils that are arrayed along the long axis of the sheet spread in parallel with the fibrils of the sheet, forming a muscle construct of cells arrayed and layered in a pattern of organization similar to that present in vivo. This basic design can be adapted to produce many different tissues, including but not limited to skeletal muscle and cardiac muscle. The cylindrical tissue construct is then implanted or placed within a RCCS bioreactor. Rates of rotation to maintain this type of construct in suspension range from 4-20 rpm, depending upon the over mass of the tissue and the specific components used to fabricate the outer cylinder.

Vascularization of the sealants and constructs containing them, occur in situ several days after surgery. In some embodiments, neovascularization of an engineered construct containing electroprocessed material is enhanced by mixing endothelial cells into the construct during fabrication. Another alternative for supplying engineered tissue containing electroprocessed material with a vascular supply is to temporarily transplant the tissue into the omentum. The sealant is removed from a bioreactor, wrapped in the omentum and supported by the diffusion of nutrients and oxygen from the surrounding tissue in the omentum. Alternatively, or in addition to this approach, sealant is connected directly to the endogenous vascular supply of the omentum. A blood vessel can be partially perforated or cut or left dissected free of the omentum. The sealant containing electroprocessed materials, depending upon the construct, is wrapped around the vessel. The sealant is supported by nutrients leaking from the perforated vessel or by the simple diffusion of nutrients if the vessel is left intact. Regardless of strategy, the sealant is surrounded by the omentum and its rich vascular supply. This procedure can be performed using blood vessels outside the omentum.

Constructs containing electroprocessed material, and optionally other substances, can be engineered with an endogenous vascular system. This vascular system can be composed of artificial vessels or blood vessels excised from a donor site on the transplant recipient. The sealant containing electroprocessed material is then assembled around the vessel. By enveloping such a vessel with the sealant during or after assembly of the engineered tissue, the sealant has a vessel that can be attached to the vascular system of the recipient. In this example, a vessel in the omentum, or other sealant is cut, and the vessel of the sealant is connected to the two free ends of the omental vessel. Blood passes from the omental vessel into the vascular system of the sealant, through the sealant and drains back into the omentum vessel. By wrapping the sealant in the omentum and connecting it to an omental blood vessel, the sealant is supported by the diffusion of nutrients from the omentum and the vessel incorporated into the tissue during its fabrication. After a suitable period of time the sealant is removed from the omentum and placed in the correct site in the recipient. By using this strategy the sealant containing electroprocessed material is supported in a nutrient rich environment during the first several days following removal from the bioreactor. The environment of the omentum also promotes the formation of new blood vessels in implanted tissue. This omental incubator strategy can be combined with the other strategies such as combining angiogenic factors in the material during electroprocessing. Several options are available. For example, the implanted sealants can be seeded with angioblasts and/or endothelial cells to accelerate the formation of vascular elements once the sealant is placed in situ. As another example, angiogenic peptides can be introduced into the sealant via an osmotic pump. Combinations of methods can also be used. The use of an osmotic pump permits delivery of peptides or, as noted, angiogenic peptides or growth factors directly to the site of interest in a biologically efficient and cost-effective manner. VEGF delivered to ischemic hind limbs of rabbits accelerated capillary bed growth, increased vascular branching and improved muscular performance with respect to ischemic controls. An alternative approach is to seed fully differentiated tissue constructs containing electroprocessed material with additional endothelial cells and or angioblasts shortly before they are implanted in situ.

In some embodiments, the stem cells or other cells used in a sealant are isolated from the subject, or other compatible donor requiring tissue reconstruction. This provides the advantage of using cells that will not induce an immune response, because they originated with the subject (autologous tissue) requiring the reconstruction. Relatively small biopsies can be used to obtain a sufficient number of cells to construct the implant. This minimizes functional deficits and damage to endogenous tissues that serve as the donor site for the cells.

Electroprocessed sealants can also be used in connection with other matrix building processes. For example, an extruded tube can have an outside layer electrospun onto it wherein the different layers complement each other and provide an appropriate matrix to promote a specific type of cell growth. In some embodiments, a vascular graft comprised primarily of a collagen tube can have an electrospun layer of both fibers (such as electroprocessed collagen, fibrinogen, fibronectin, elastin, or combinations thereof) and cells added to promote the acceptability of the graft in a particular recipient. A second example is an in vitro skin preparation formed by growing fibroblasts in one layer, covering the first layer with electroprocessed material, and then growing a second layer composed of epidermal cells in the matrix. This layering technique can be used to make a variety of tissues.

### Example 1

### Electrospinning a Solution of Human Fibrinogen

Lyophilized, human fibrinogen, Fraction I from plasma (Sigma-Aldrich Chemical Co.). was suspended in a solution composed of 8 parts HFP (Sigma-Aldrich Chemical Co.) and 1 part 10× minimal essential medium (MEM), Earle's (without L-glutamine and sodium bicarbonate) at a concentration of 0.083 grams/ml HFP/MEM. Once in solution or suspension, the fibrinogen solution was loaded into a 1.0 ml syringe. An 18-gauge stub (blunted) needle was then placed on the syringe to act as the electrospinning nozzle and charging point for the contained fibrinogen solution. The filled syringe was placed on a KD Scientific syringe pump using a Becton-Dickinson 1.0 ml Plunger set to dispense the solution at a rate of 1.85 ml/hr. The positive lead from the high voltage supply was attached to the metal stub of the syringe. The syringe pump was turned on and the high voltage supply was set at 22 kV. The grounded target was a 303 stainless steel mandrel (0.1 cm W×0.6 cm H×2 cm L) placed five inches from the tip of the needle. The mandrel was rotated at approximately 3500 rpm. The fibrinogen solution was electrospun to form a white mat on the grounded mandrel. After electrospinning (0.4 ml total volume), the mat was removed from the mandrel and processed for scanning (SEM) and transmission (TEM) electron microscopy evaluation.

SEM of the electrospun material revealed a scaffold composed of fibers with an average diameter of 80±30 nm. The mat produced in this feasibility study was approximately 100 μm thick. The 80 nm fibers are in the reported range (82-91 nm) for the mean diameter of fibrin in plasma clots. TEM evaluation revealed that the 80 nm fibers had a typical, granular appearance with 22.5 nm banding, which is characteristic of the native fibrinogen as present in clots. The electrospun mats possessed substantial structural integrity, which allowed them to be removed with care from the mandrel and handled. The electrospun mat produced was hydrophobic at first but wetted quickly in a normal saline solution. The electrospun mat was also insoluble in normal saline and remained intact as a hydrated mat for at least 24 hours.

### Example 2

### Electrospinning a Solution of Human Fibrinogen

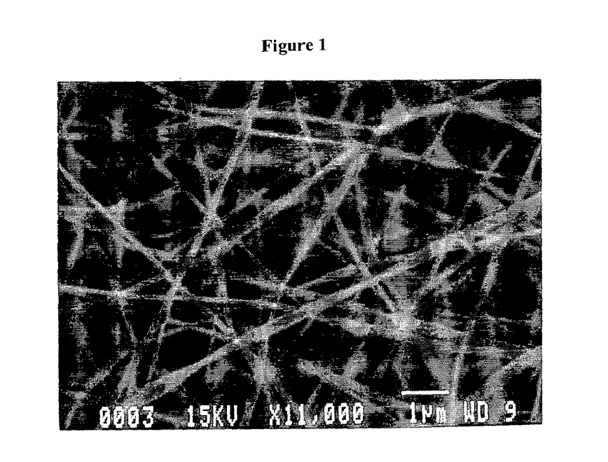

Human fibrinogen, Fraction I from plasma (Sigma, Cat # F-4883) was suspended in a solution composed of 8 parts HFP and 1 part 10×MEM Earles (without L-glutamine and sodium bicarbonate). 0.075 grams of fibrinogen were used in 0.9 ml HFP/MEM. Once in solution or suspension (milky, yellow color), the solution was loaded into a 1.0 ml syringe. A 18-gauge stub (blunted) needle was then placed on the syringe to act as the electrospinning nozzle and charging point for the contained fibrinogen solution. The filled syringe was placed in the KD Scientific's syringe pump set to dispense the solution at rate of 1.88 ml/hr utilizing a Becton Dickinson 1.0-ml syringe plunger. The positive lead from the high voltage supply was attached to the stub of the metal portion of the syringe. The syringe pump was turned on and the high voltage supply turned on and set at 21 kV. The grounded target was a 303 stainless steel mandrel (0.6 cm W×0.05 cm H×4 cm L) placed approximately 4 inches from the tip of the adapter. The mandrel was rotated at a speed less than 3500 rpm. In the experiment, the fibrinogen solution was electrospun to form a white mat on the grounded mandrel. After electrospinning, the mat was removed from the mandrel and processed for scanning electron microscopy evaluation (FIG. 1). The mat produced was approximately 100 μm thick.

### Example 3

### Electrospinning a Solution of Bovine Fibrinogen

Bovine fibrinogen, Fraction I, Type I-S from plasma (Sigma, Cat # F-6630) was suspended in a solution composed of 8 parts HFP and 1 part 10×MEM Earles (without L-glutamine and sodium bicarbonate). 0.233 grams of fibrinogen were used in 2.7 ml HFP/MEM. Once in solution or suspension (milky, yellow color), the solution was loaded into a 3.0 ml syringe. A 18-gauge stub needle was then placed on the syringe to act as the electrospinning nozzle and charging point for the contained fibrinogen solution. The filled syringe was placed in the KD Scientific's syringe pump set to dispense the solution at a rate of 1.88 ml/hr utilizing a Becton Dickinson 1.0-ml syringe plunger. The positive lead from the high voltage supply was attached to the stub adapter metal portion. The syringe pump was turned on and the high voltage supply turned on and set at 21 kV. The grounded target was a rotating 303 stainless steel mandrel (0.5 cm W×1.0 cm H×7.5 cm L) placed approximately 4 inches from the tip of the adapter. In the experiment, the fibrinogen solution was electrospun to form a white mat on the grounded mandrel. After electrospinning, the mat was removed from the mandrel and processed for scanning electron microscopy evaluation. The resulting matrix had a soft, elastic and pliable texture. The mat produced was approximately 70 μm thick.

The scaffold produced on the mandrel had significant mechanical integrity. As an example, one end of the produced scaffold was lifted from the mandrel after spinning and the rest of the length (˜7 cm) was removed by pulling on the excised end.

### Example 4

### Electrospinning a Solution Containing a Blend of Fibrinogen and Collagen

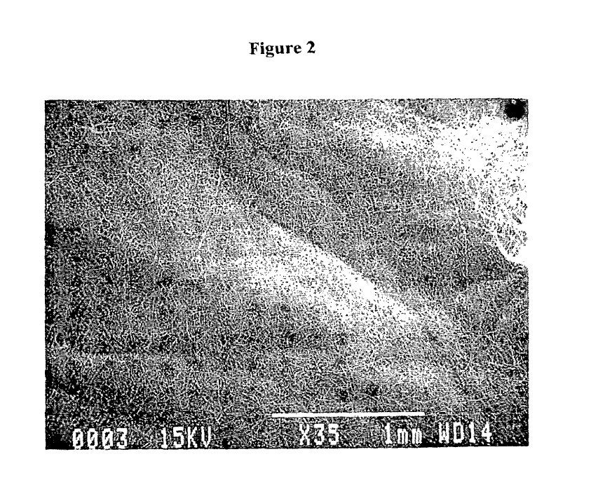

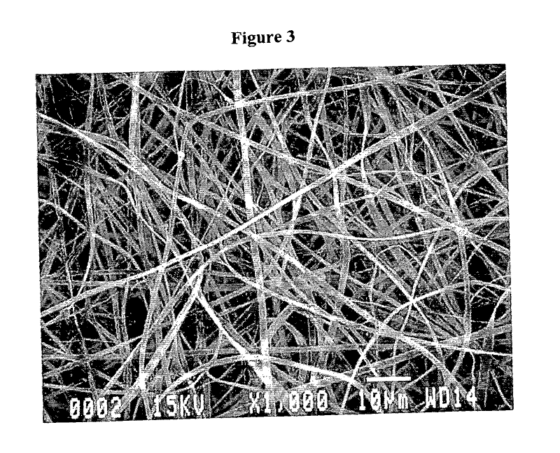

Bovine fibrinogen, Fraction I from plasma (Sigma, Cat # F-4883) and Type I collagen (calf skin, Sigma Chemical Co. No. 3511) were electrospun together from HFP. The fibrinogen and collagen were blended in a solution or suspension composed of 9 parts HFP and 1 part 10×MEM Earles (without L-glutamine and sodium bicarbonate). 0.105 grams of fibrinogen and 0.077 grams of collagen were used in 1.0 ml HFP/MEM. Once in solution or suspension (milky, yellow color), the liquid was loaded into a 1.0 ml syringe plunger. A 18-gauge stub needle was then placed on the syringe to act as the electrospinning nozzle and charging point for the contained fibrinogen/collagen solution. The filled syringe was placed in the KD Scientific's syringe pump set to dispense the solution at rate of 2.34 ml/hr utilizing a Becton Dickinson 1.0-ml syringe plunger. The positive lead from the high voltage supply was attached to the stub adapter metal portion. The syringe pump was turned on and the high voltage supply turned on and set at 22 kV. The grounded target was a 303 stainless steel mandrel (0.6 cm W×0.05 cm H×4 cm L) placed approximately 5 inches from the tip of the adapter. The mandrel was rotated at approximately 3500 rpm during spinning. The fibrinogen/collagen was electrospun to form a white mat on the grounded mandrel. After electrospinning, the mat was removed from the mandrel and processed for scanning electron microscopy evaluation. The results of this fibrous mat production can be seen in FIGS. 2 and 3. The mat produced was approximately 500 μm thick. The resulting matrix had a softer, more elastic and more pliable texture than that of electrospun collagen and was not soluble in water, 1×, or 10×MEM Earle's salt solution (without L-glutamine and sodium bicarbonate) for at least 24 hours.

The same suspension or solution was then electrospun onto a 4 mm ID cylindrical tube. Parameters for spinning were the same except that the mandrel was rotated at approximately 6000 rpm around the long axis of the cylinder. A micrographs of the resulting matrix is shown in FIG. 4. These matrices show alignment of the fibrous structure.

### Example 5

### Electrospinning Fibrinogen Solutions Having Different Concentrations

A 9:1 solution of HFP to 10×MEM was mixed and ⅙^{th} (0.167 g/ml), ⅛^{th}(0.125 g/ml), and 1/10^{th} (0.100 g/ml) concentration solutions with bovine fibrinogen were made. Each solution was electrospun using the parameters set forth in Example 1 except that the distance was 2 inches between the needle tip and the mandrel.

The ⅙^{th} weight by volume solution of fibrinogen produced a mat that was fibrous and easy to remove from the mandrel for mechanical testing and SEM analysis. The ⅛^{th} weight by volume solution of fibrinogen was much easier to spin in that it was less prone to clogging the spinning orifice and produced a fibrous mat that was also easy to remove from the mandrel for mechanical testing and SEM. The 1/10^{th} weight by volume solution of fibrinogen spun most easily with minimal clogging, but the mat was thin and could not be removed from the mandrel without tearing. Thus, it was not mechanically tested and was only observed with the SEM. The ⅙^{th} weight by volume solution of fibrinogen an average fiber diameter of 700 nm and an average pore size of 46.69 μm². The ⅛^{th} weight by volume solution of fibrinogen had an average fiber diameter of 310 nm and an average pore size of 14.41 μm². The 1/10^{th} weight by volume solution of fibrinogen had an average fiber diameter of 330 nm and an average pore size of 11.36 μm².

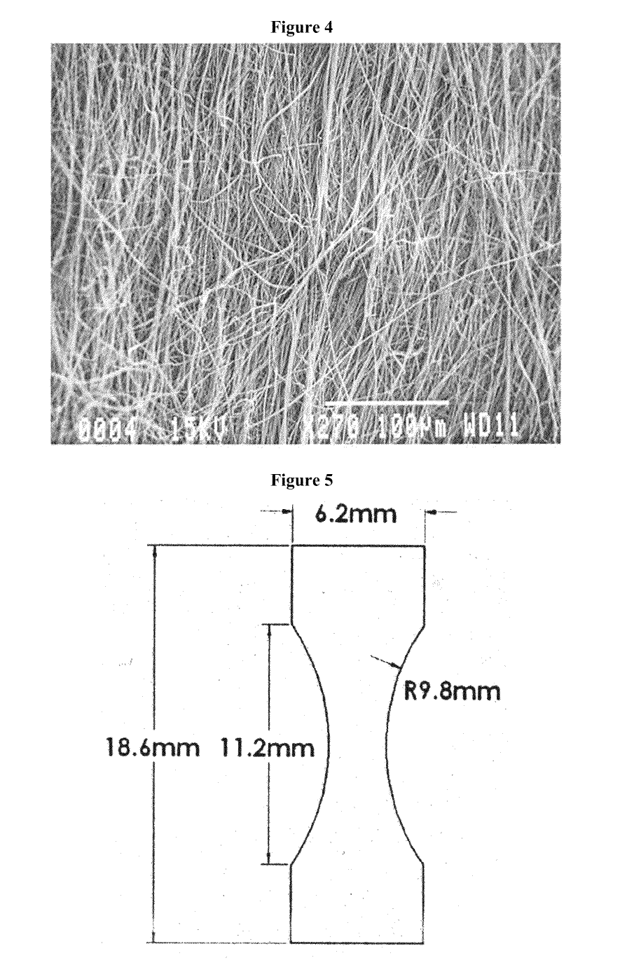

By cutting the mats of the ⅙^{th} and ⅛^{th} weight by volume solutions of fibrinogen along lines perpendicular to the direction of rotation, samples were obtained that could be tested mechanically. The bulk material mechanical properties including the Young's modulus (referred to as “Elastic Modulus” in tables below), and ultimate tensile strength (referred to as Peak Stress in the tables below) of the scaffolds produced was determined by tensile testing (stress-strain relationship data). For this data, the electrospun scaffolds were subjected to stress-strain analysis using a MTS Bionix 200 materials testing station (MTS Systems Corp.; Eden Praire, Minn.). The samples were trimmed into a “dog-bone” profile (FIG. 5) with offset ends to reduce grip effects and provide uniformity across samples. The testing was conducted with the tissue grips moving at a rate of 10 mm/min. The data acquisition rate was set to 20.0 Hz. The data integration and analysis was completed using the MTS Testworks software (version 4.04A). The inputs for each test were the gage, thickness, and width of each sample. Results are presented in Table 1 and Table 2.

TABLE 1
1/6^{th} Concentration Mechanical Testing Results

| | | | Elastic |
|---|---|---|---|
| 1/6 concentration | Peak Load (N) | Peak Stress (MPa) | Modulus (Mpa) |
| Sample 1 | 0.219 | 2.700 | 81.103 |
| Sample 2 | 0.277 | 2.700 | 127.280 |
| Sample 3 | 0.231 | 2.600 | 39.574 |
| Sample 4 | 0.239 | 1.000 | 64.443 |
| Sample 5 | 0.141 | 1.000 | 85.534 |
| Sample 6 | 0.234 | 2.600 | 61.577 |
| Sample 7 | 0.235 | 0.700 | 72.338 |
| Sample 8 | 0.274 | 3.400 | 171.349 |
| Sample 9 | 0.270 | 4.000 | 53.234 |
| Average | 0.236 | 1.856 | 78.133 |

TABLE 2
1/8^{th} Concentration Mechanical Testing Results

| | | | Elastic |
|---|---|---|---|
| 1/8 concentration | Peak Load (N) | Peak Stress (MPa) | Modulus (Mpa) |
| Sample 1 | 0.213 | 1.200 | 6.600 |
| Sample 2 | 0.226 | 1.700 | 28.180 |
| Sample 3 | 0.278 | 2.000 | 13.455 |
| Sample 4 | 0.230 | 1.600 | 30.449 |
| Sample 5 | 0.311 | 2.300 | 26.235 |
| Average | 0.252 | 1.76 | 20.863 |

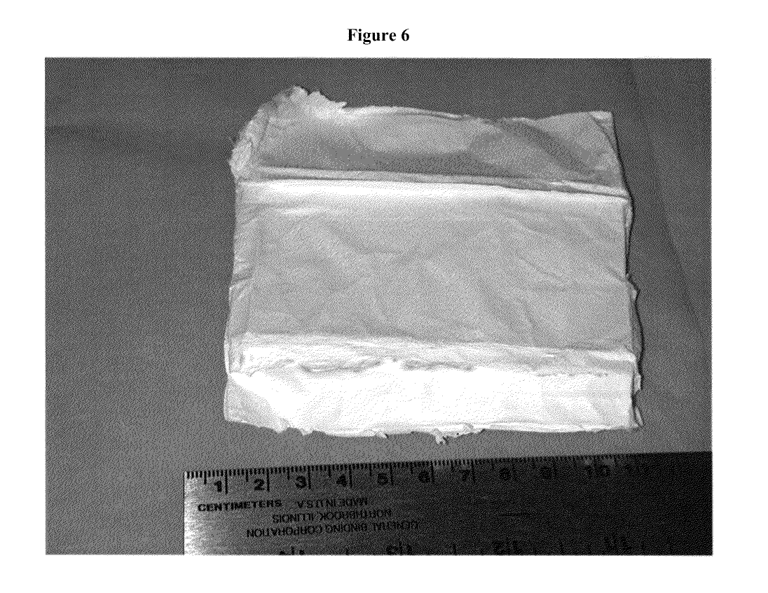

A mat (shown in FIG. 6) was spun from a ⅙^{th} weight by volume solution, having with a mass of 0.0778 g, average thickness of 0.0263 in (0.6680 mm), and length and width of 10 cm by 10 cm. A smaller mat was cut from this larger mat with length and width of 66.5 mm and 59.0 mm. These dimensions give a volume of 2620.9 mm³.

Another aspect of the electrospun materials is the high surface area to volume ratio. This is an important property in some embodiments involving a hemostatic product such as a bandage in which the rate and extent of the coagulation in contact with the bandage in some embodiments are directly related to the surface area available for reaction with the blood components and thereby form a clot or other seal. Using the sheet spun from ⅙th weight by volume solution of fibrinogen as an example, the 700 nm average fiber diameter and the 1.38 g/cm² density of fibrinogen provides an estimated total surface area of the fibers of 3,300 cm². With sheet dimensions of 60 mm×60 mm×0.7 mm, the fiber surface area to volume ratio is 1,300 cm²/cm³. The dry mass of this sheet is approximately 0.08 grams, so that the relative surface area to weight ratio is 41,000 cm2/g.

### Example 6

### Use of Electrospun Sealants on Skin Wounds

Acid soluble Type I collagen isolated from calfskin (Sigma part number 3511), the commercial product VITROGEN 100 (Cohesion Tech, Inc. of Palo Alto, Calif.), and gelatin (Sigma) were prepared for electrospinning. The Type I collagen was re-extracted in ice cold 0.01 N HCL overnight and dialized against 10 volumes of ice cold ultrapure water with three changes of water at 24 hour intervals for a three day period. VITROGEN was purchased as a solution of collagen in 0.01 N HCl and was dialized directly against 10 volumes of ice cold ultrapure water with three changes of water at 24 hour intervals for a three day period. VITROGEN 100 is Bovine collagen Type I isolated from skin. VITROGEN is a commercially available acid soluble extract of calfskin that has been subjected to a pepsin digest and lacks the telopeptides that are characteristic of natural collagen. Dialized Type I collagen from Sigma and VITROGEN were each frozen at −70 degrees C. and lyophilized to a dry powder.

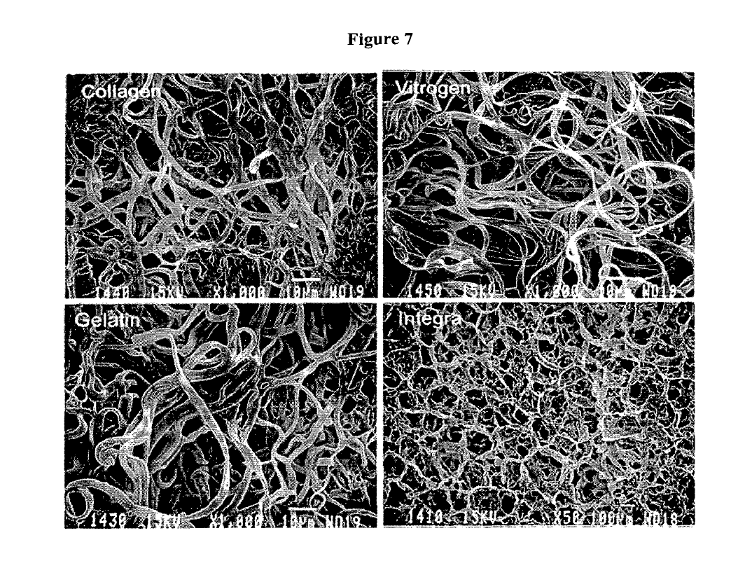

Lyophilized Type I collagen and VITROGEN were then each separately dissolved in HFP (80 mg/ml) for electroprocessing. Dry lyophilized gelatin pellets (Sigma Adrich #G-9391) were solubilized at 80 mg/ml overnight in HFP at 80 mg/ml. Conditions were adjusted to deposit Type I collagen, VITROGEN and gelatin into separate nonwoven matrices composed of 1-5 μm diameter fibers. Collagen solutions/suspensions were charged to 18-20 kV and directed at a rotating, grounded rectangular mandrel (approximately 40 mm×100 mm) across a distance of five inches. The mandrel was rotated at an approximate speed of 3500 rpm or less. Constructs 100-150 μm in diameter were prepared from Type I collagen (from Sigma collagen). The same procedures were repeated for the VITROGEN and the gelatin. On average, these constructs were composed of fibers that ranged from 1-5 μm in diameter. At the conclusion of electrospinning, the mats were vapor fixed in glutaraldehyde for 12 hours in small sealed chambers. FIG. 7 illustrates scanning electron micrographs of electrospun collagen, electrospun VITROGEN, and electrospun gelatin and INTEGRA Dermal Regeneration Template, a non-electrospun collagen product sold for skin repair by Integra LifeSciences, Plainsborough, N.J. It is composed of collagen aggregates and exhibits a large open cell structure. Each of the electrospun materials deposit as a non-woven matrix composed of filaments that range from 1-5 μm in diameter. Note the size bar in the panel depicting INTEGRA indicates that the image was captured at a substantially lower magnification than the accompanying images. INTEGRA is a freeze dried collagen sponge containing glycosoaminoglycans from shark cartilage and having a silicone backing. Each of the three electrospun materials exhibited differing chemical, physical and biological properties. Dry electrospun Type I collagen had a stiff and relatively inelastic texture, electrospun VITROGEN was softer and much more pliable, while electrospun gelatin was more elastic than either of the other electrospun materials.

A guinea pig model was used to investigate the efficacy of using electrospun materials in the reconstruction of dermal injuries. Guinea pigs were anesthetized, and a set of four, full-thickness dermal wounds (1 cm²) was prepared on the dorsum of each animal. Sheets of electrospun Type I collagen, VITROGEN, gelatin or INTEGRA were immersed in 0.1 M glycine to block in any unreacted glutaraldehyde, and then rinsed several times in sterile PBS supplemented with PenStrep antibiotics (Gibco) and cut to fit the injury sites. Each scaffolding was covered with a silver impregnated dressing and sutured in place. A bolster was fitted over the entire injury site to maintain gentle pressure on the dressings and inhibit wound contraction. At intervals the animals were sacrificed and the tissue was recovered for histological evaluation.

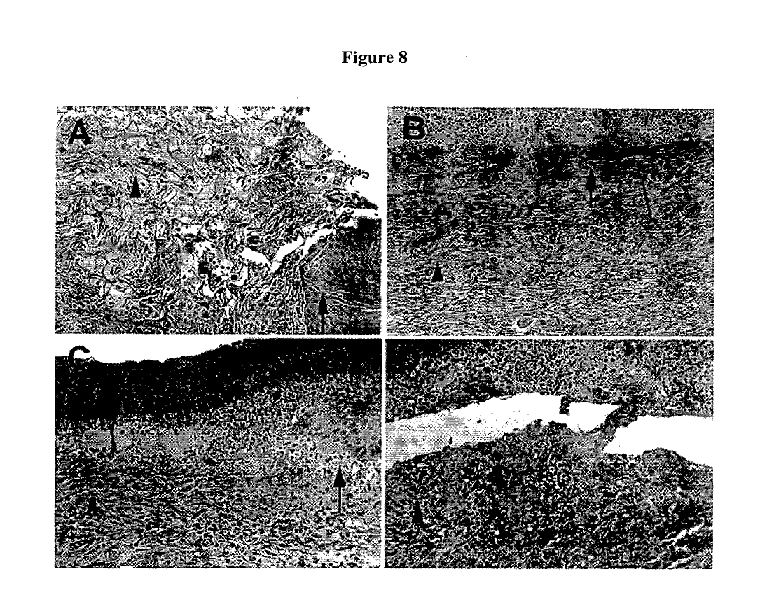

Images in FIG. 8 depict (magnification approximately 10-20×) the interface of the prosthetics and the surrounding healthy tissue at the margin of the wound after 7 days. After 7 days, the following observations were made:

(A) INTEGRA. (FIG. 8, Panel A) The arrowhead in panel A marks a domain within the INTEGRA matrix. Implant was poorly infiltrated with dermal fibroblasts. Cells were scattered at a very low density. At the margin, formation of the tongue (an extension of healthy epidermis across the wound site, which marks the early stages of healing) was limited or nonexistent. Picnotic nucluei were present within the INTEGRA collagen sponge. The large open structure of INTEGRA was clearly evident throughout the implant site. Picnotic nuclei (N, arrow) and inflammatory cells were scattered throughout the matrix.

(B) Electrospun collagen. The tongue was fully established at the margin of injury in wounds treated with electrospun collagen. (FIG. 8, Panel B) The formation of the epithelial tongue represents an important landmark in the healing of the epithelium and is a reflection of how readily epithelial cells can migrate across the surface of a wound bed. This dermal matrix was densely infiltrated throughout with fibroblasts (arrowheads) that exhibited an elongated, fusiform shape. Granulation tissue was evident on the dorsal surface of the wound across the entire wound bed. Functional blood vessels were present within the matrix.

(C) Electrospun VITROGEN. (FIG. 8, Panel C). Scaffolds of electrospun VITROGEN also were densely populated with elongated dermal fibroblasts (arrowheads). At the margin of the injury, tongue formation was well established. Functional blood vessels were present within the matrix. Granulation tissue covered the entire wound site. Dorsal border of scaffold is marked by arrowheads.

(D) Electrospun gelatin. (FIG. 8, Panel D). Electrospun gelatin appeared to induce an inflammatory response and extensive inflammation and edema were present subjacent to the margin in wounds treated with this type of matrix Lymphocytes and picnotic nuclei were scattered throughout this matrix. Inflammatory cell infiltration is illustrated with (triple asterisks, (***)). Tongue formation was evident, but was not as extensive as in the other electrospun scaffolds.

After 14 days the following observations were made:

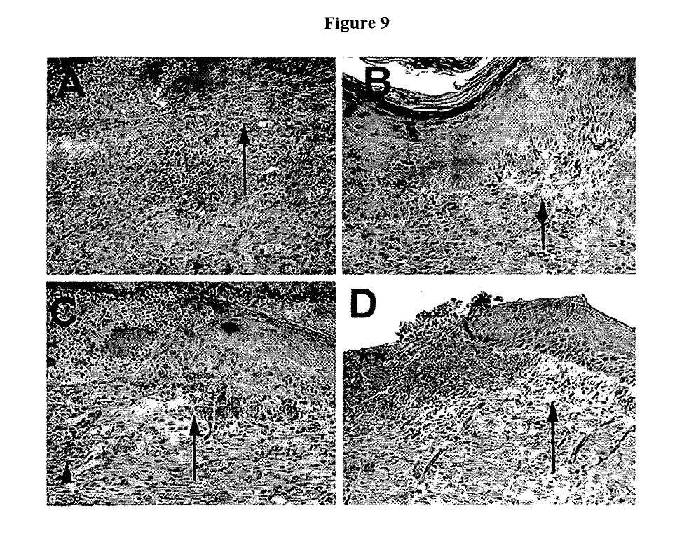

(A) INTEGRA. Implants were infiltrated with dermal fibroblasts and tongue formation was evident at the margin of the injury site (FIG. 9, Panel A). The fibroblasts in the INTEGRA were scattered throughout the implanted matrix and did not exhibit a high degree of alignment. The large, open pores present in INTEGRA were evident even after 14 days in vivo. Modest tongue formation was evident, but not was not as extensive as in the electrospun scaffolds. Residual inflammatory cells are present at low concentration.

(B) Electrospun collagen. Dermal injuries treated with sheets of electrospun collagen were densely infiltrated with dermal fibroblasts and exhibited a nearly continuous layer of epithelial cells. (FIG. 9, Panel B, arrow). This epithelial layer lacked rete pegs (a histological feature of mature skin), but was continuous across the injury. The epidermis was multilayered and exhibited a well differentiated phenotype. A dense cell population appeared throughout the scaffold. The arrow in FIG. 9, Panel B marks the transition between uninjured epithelium and regenerated tissue. These data suggest that electrospun collagen supports very rapid epithelial cell migration.

(C) Electrospun VITROGEN. Implants (FIG. 9, Panel C) were extensively vascularized and had large and well established tongues of epithelium at the margins. Scaffolds were densely infiltrated with dermal fibroblasts and functional capillary networks are found (arrows).

(D) Electrospun gelatin. (FIG. 9, Panel D) Implants continued to exhibit evidence of edema (double asterisk, (**)) and inflammation. Functional blood vessels were present. Picnotic nuclei and inflammatory cells were scattered throughout the matrix. Limited tongue formation was evident (arrow), but not was not as extensive as in the other electrospun scaffolds.

### Example 7

### Sealants with Aligned Collagen Fibrils

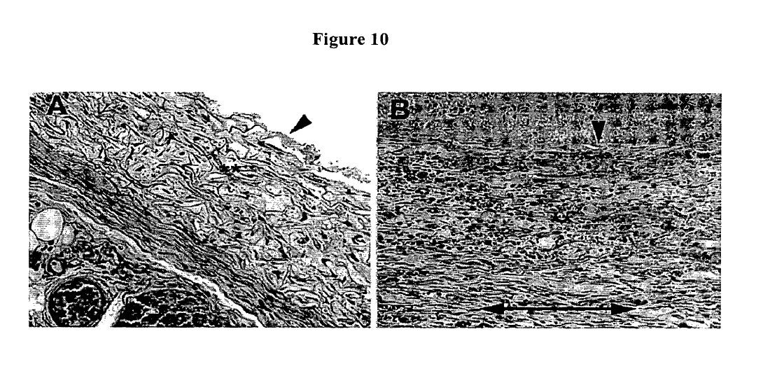

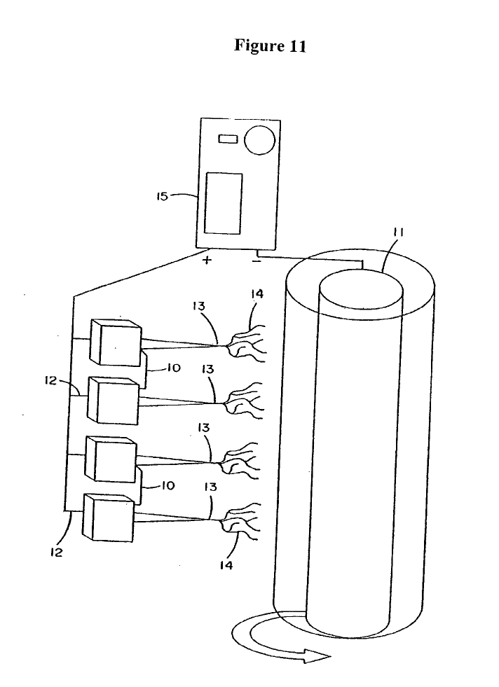

A matrix composed of collagen fibrils aligned along a common axis was produced. This structural property is used to accelerate the alignment of dermal fibroblasts within a wound site. Electrospun collagen sheets were made using the same materials and parameters of Example 6 above except that the mandrel was rotated at approximately 5000-6000 rpm. The sheets were then applied to guinea pig skin wounds using the same procedures set forth in Example 6 above. FIG. 10 shows micrographs (20×) of the wound after seven days. Images were captured in the middle of the injury site just subjacent to free surface of implants (arrowheads denote free surface). The substance resting on the electrospun matrix of collagen is granulation tissue; this substance was lost during processing from the sample treated with INTEGRA. Two observations are evident. First, after seven days in a full thickness dermal wound an INTEGRA-based implant is poorly infiltrated by cells (Panel A, double asterisk (**)), while electrospun collagen is densely populated in a similar domain over the same time course (Panel B, double asterisk (**)). Second, cells within INTEGRA are scattered at random throughout the matrix. Within a matrix of electrospun collagen the dermal fibroblasts are aligned in parallel with the surrounding collagen fibrils (Panel B, arrow).

### Example 8

### Use of Sealants as a Hemostatic Agent

Adult Sprague Dawley Rats (500-700 gms) were anesthetized with ketaset (80-180 mg/kg). A mid-line incision was made in the abdominal wall. Hemostatic devices were tested on three separate sites, the liver, the spleen and the abdominal aorta. No more than one organ site was tested per animal.

For testing on the liver and spleen, a small tangential slice was prepared on the surface of the tissues. At incision these organs oozed blood at low pressure. Portions of the electrospun sheet prepared in EXAMPLE 5 from the ⅙ concentration solution were applied. For liver and spleen injuries the electroprocessed material wet by absorbing fluid and appeared to shrink (contract) onto the wound site. Bleeding was suppressed within an estimated 5-15 seconds. Electrospun sheets of PGA approximately 200 μm thick (spun from a 100 mg/ml PGA in HFP, using a potential of 23 kV with an air gap of 6 inches separating the source solution from the ground target; PGA solution was delivered at about 5 mL per hour) did not wet when applied to this type of wound and did not appear to suppress bleeding. On the liver and spleen hemostasis was most effectively achieved with mat electrospun from fibrinogen followed by the mats electrospun from collagen and then PGA.

For testing the abdominal aorta, internal organs were dissected free and moved to the side to expose the abdominal aorta. Fascia and adherent fat were cleared from the great vessels and a 23 gauge needle was used to puncture the aorta. When the needle was removed from the vessel a jet of blood was observed that pulsed with each contraction of the heart. When a sheet electrospun from fibrinogen (approximately 1 cm by 1 cm) was placed onto this type of injury, it wet almost immediately and contracted onto the injury site. Excess blood that had pooled in the abdominal cavity was blotted with gauze and gentle pressure was applied by hand (fingertip) to the surface of the patch. When the pressure was relieved from the injury site blood was visible oozing outward from underneath the patch site. A second sheet of the same composition and dimensions was placed over this adjacent site; pressure was reapplied for 5-10 seconds and reduced oozing further. A third patch of the same composition and dimensions was placed over the site and bleeding ceased.

After 30-60 seconds a second puncture wound was prepared distal to the initial injury site. Arterial blood flow was evident from this puncture, demonstrating the patency of the aortic tree following treatment with the patch.

In some animals, aorta puncture resulted in blood leaking a slower rate (similar to an ooze rather than a jet of blood). When a single patch of the electrospun fibrinogen was placed onto this type of injury site (1×1 cm square and 300-400 μm thick) bleeding was stopped with the single sheet.

Sheets of electrospun Type I collagen (0.1 g/ml TFE, 5 inch air gap, 23 kV, rotation approximately 1000 rpm, dispensing speed 5 ml/hr) composed of fibers having an average fiber diameter of about 750 nm in a 2 in×2 in sheet 350 μm thick were also tested and also suppressed bleeding, although not as rapidly as the sheets of electrospun fibrinogen. A sheet of electrospun collagen applied to a spleen injury wetted nearly immediately and conformed to the shape of injury of the spleen and suppressed bleeding. Similar results were obtained with injuries to the liver. However, sheets of electrospun collagen were ineffective at stopping injuries to the abdominal aorta where blood was freely spurting from the vessel.

Sheets of electrospun PGA approximately 200 μm thick (parameters same as those noted earlier in this example were also used. When this material was applied it appeared to absorb blood much more effectively than it did when it was placed onto the liver or spleen (much less fluid in these sites). Several sheets were applied; bleeding was much more extensive than with the patch of electrospun fibrinogen, however evidence that clotting initiated was observed. The wettability of PGA can be enhanced by acid pretreatment (for example, by immersing in 12 M HCL for 5 minutes) or by wetting in 70% alcohol for a few minutes prior to immersion in water.

### Example 9

### Hemostatic Agents with Higher Concentrations

Adult Sprague Dawley Rats (500-700 gms) were anesthetized with ketaset (80-180 mg/kg) and the procedures relating to the abdominal aorta in Example 8 were repeated except that the mats of electrospun fibrinogen were 300-500 μm thick. As in Example 8 the abdominal aorta was exposed and punctured with a 23 gauge needle. When the needle was withdrawn a jet of arterial blood spurted from the wound site. A single sheet of electrospun fibrinogen (2 cm in length×1.2 cm in width×300-500 μm thick) was applied over the injury and compressed for 10 seconds with gentle pressure. The injury remained sealed after releasing pressure for 20 seconds, and the heart continued to contract vigorously. A small amount of seepage of blood was observed under one edge of the sheet. Additional pressure was applied to that site for 10 seconds with a fingertip, and all bleeding stopped. After an additional minute the sheet was removed. A clot was evident around the aorta in the injury site and no additional bleeding was evident even after removal of the sheet. Puncturing the Aorta distal to the initial injury site resulted in a fresh jet of arterial blood. This jet of blood demonstrates the patency of the vessel and confirms that perfusion pressures at the site of the clot were substantial and sufficient to support vigorous bleeding if the original injury site had not been completely sealed by the treatment.

### Example 10

### Sealants with Additional Substances to Assist Coagulation

A matrix of electrospun fibrinogen is prepared as described in Example 1 above and an matrix of electrospun fibrinogen and collagen is prepared as described in Example 2. Calcium chloride, thrombin, factor XIII and aprotinin are applied to each matrix by aerosol spraying one or more solutions containing these substances upon each matrix, brush application of the substances, or by immersing each matrix into solutions containing these substances. The resulting matrices are applied to sites at which formation of a clot or seal is desired. Alternatively, the matrices are applied to the sites and the substances are subsequently applied to the matrix by spraying or brush application. For sites located inside the body of an organism, an endoscope is used to facilitate application.

### Example 11

### Sealants with Electrospun Compositions from Separate Nozzles

An electrospun matrix is prepared by spinning a solution of fibrinogen as described in Example 1 above and simultaneously electrospinning Type I collagen from a separate nozzle onto the same mandrel, substrate, or target to form a matrix of fibers. Calcium chloride, thrombin, Factor XIII and aprotinin are applied to the matrix by aerosol spraying one or more solutions containing these substances upon the matrix, brush application of the substances, or by immersing the matrix into solutions containing these substances. The resulting matrix contains each of these components and is applied to a site at which formation of a clot is desired. Alternatively, the matrix is applied to the site and the substances are subsequently applied to the matrix. The resulting matrices are applied to sites at which formation of a clot or seal is desired.

### Example 12

### Sealants with Electrospun Substances

Electrospun matrices are prepared as described in Example 11 except that thrombin is electroprocessed along with the collagen such that the thrombin is associated with the fibers in the resulting matrix. The procedure is then repeated except that aprotinin is added to the electrospinning solution for fibrinogen instead of being applied after electroprocessing such that instead of being applied after electroprocessing the aprotinin is associated with the fibers in the resulting matrix. The procedure is repeated again except that Factor XIII is electrospun along with the solution of fibrinogen and aprotinin instead of being applied after electroprocessing so that Factor XIII and aprotinin are associated with the fibers in the resulting matrix. The procedure is repeated again with the difference that calcium chloride is electrospun along with the collagen and thrombin instead of being applied after electroprocessing. The procedure is then repeated such that all components of the matrix are electroprocessed, with some substances (Factor XIII and aprotinin) being in the fibrinogen electroprocessing solution, and the remaining substances (thrombin and calcium chloride) being in the collagen electroprocessing solution. The resulting matrices are applied to sites at which formation of a clot or seal is desired. In many cases, however, it is preferred to electrospin the fibrinogen or to apply fibrinogen by some other process using solutions separate from that containing thrombin or Factor XIII.

### Example 13

### Sealants with Substances Added by Electrospraying

Each of the procedures in Example 10 and Example 11 are each repeated with the difference that the thrombin, aprotinin, Factor XIII, and calcium chloride are applied to the electrospun matrix by an electrospray process rather than by aerosol spraying or dipping. The resulting matrices are applied to sites at which formation of a clot or seal is desired.

### Example 14

### Sealants with Fibronectin

Each of the procedures of Example 10, 11, 12, and 13 are repeated with the difference that fibronectin is also electrospun from the solution that contains the fibrinogen. Each of the procedures of Example 10, 11, 12, and 13 are then repeated except that fibronectin is electrospun along with collagen from the solution that contains the collagen. Each of the procedures of Example 10, 11, 12, and 13 are then repeated except that fibronectin is applied to the electrospun matrix by electrospraying. Each of the procedures of Example 10, 11, 12, and 13 are then repeated except that the fibronectin is applied to the matrix by aerosol spraying one or more solutions containing fibronectin upon each matrix or by immersing each matrix into solutions containing fibronectin. The resulting matrices are applied to sites at which formation of a clot or seal is desired.

### Example 15

### Sealants with a Fibrinolytic Inhibitor

Each of the procedures of Example 10-14 are repeated with the difference that Thrombin-Assisted Fibrinolytic Inhibitor (TAFI) is also electrospun from the solution that contains the fibrinogen. Each of the procedures of Example 10-14 are then repeated except that TAFI is electrospun along with collagen from the solution that contains the collagen. Each of the procedures of Example 10-14 are then repeated except that TAFT is applied to the electrospun matrix by electrospraying. Each of the procedures of Example 10-14 are then repeated except that the TAFI is applied to the matrix by aerosol spraying one or more solutions containing TAFI upon each matrix or by immersing each matrix into solutions containing TAFI. The resulting matrices are applied to sites at which formation of a clot or seal is desired.

### Example 16

### Electrospinning a Blend of Collagen and Thrombin

Approximately 100 NIH units of bovine thrombin (Sigma Chemical Co.) was dissolved in 0.1 mL 10×MEM Earle's (without L-glutamine and sodium bicarbonate). About 0.9 mL of HFP (Sigma-Aldrich Chemical Co.) was added in addition to 0.08 g bovine collagen. The material was mixed until dissolved and loaded into a 1.0 ml syringe. An 18-gauge stub (blunted) needle was then placed on the syringe to act as the electrospinning nozzle and charging point for the contained collagen solution. The filled syringe was placed on a KD Scientific syringe pump using a Becton-Dickinson 1.0 ml Plunger set to dispense the solution at a rate of 1.85 ml/hr. The positive lead from the high voltage supply was attached to the metal stub of the syringe. The syringe pump was turned on and the high voltage supply was set at 22 kV. The grounded target was a 303 stainless steel mandrel (0.1 cm W×0.6 cm H×2 cm L) placed five inches from the tip of the needle. The mandrel was rotated at approximately 3500 rpm. The collagen-thrombin solution was electrospun to form a white mat on the grounded mandrel.

### Example 17

### Application of Thrombin to Electrospun Collagen

An electrospun collagen matrix was made by electroprocessing a solution having a concentration of 0.08 g/ml bovine Type I collagen in HFP. The collagen suspension or solution was placed into a syringe. The filled syringe was placed on a KD Scientific syringe pump using a Becton-Dickinson 1.0 ml Plunger. The positive lead from the high voltage supply was attached to the metal stub of the syringe. The syringe pump was turned on and the high voltage supply was set at approximately 23 kV. The target was a stainless steel mandrel disposed about 6 inches from the end of the needle. The target was rotated at approximately 3500 rpm and was rectangular. The faces upon which the electroprocessed materials was spun were about 1×3 inches in diameter. Approximately 2 mL of solution was spun. The electroprocessed material was removed from the mandrel and part of one face of electroprocessed material (a portion about 1×1.5 inches in size) was taken for further processing.

The portion of the electrospun matrix was placed into a petri dish. Approximately 40 NIH units of Bovine thrombin (Sigma Chemical Co.) were suspended in water and applied to the mat using an airbrush. The thrombin suspension was sprayed onto the collagen mat until the mat had a saturated appearance. The mat was then left in a pool of the thrombin suspension in the petri dish. The electroprocessed material was allowed to dry overnight at 4° C. The electroprocessed material was then placed into about 0.04 mL of phosphate-buffered saline (PBS) and stirred at room temperature for a period of 15 minutes. The collagen mat was then pelleted by centrifugation and the PBS-thrombin solution was withdrawn. Thrombin activity within the PBS was confirmed by a spectrophotometric assay using D-Phe-L-Pipecolyl-Arg P-Nitroanilide, a colorimetric enzymatic substrate.

### Example 18

### Relationship Between Fibrinogen Concentration and Fiber Diameter

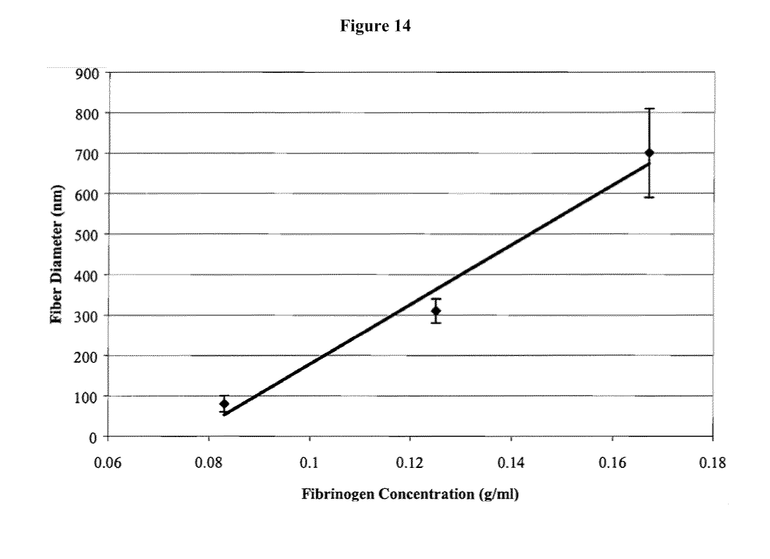

To demonstrate the control of fiber diameter by varying the fibrinogen solution, bovine fibrinogen solutions were electrospun at concentrations of 0.083, 0.125 and 0.167 g/ml in the HFP/MEM with all other process parameters maintained constant. The results of this processing resulted in 80±20, 310±70 and 700±110 nm average fiber diameters, respectively. These results are plotted in FIG. 14 to illustrate the linear relationship (R2=0.98) between fibrinogen concentration and the fiber diameters produced during electrospinning. This broad range of fiber diameters allows for tremendous flexibility in design and fabrication of sealants including, but not limited to, sealants used as tissue engineering scaffolds, wound dressings, and hemostatic products.

### Example 19

### Layered Dermal Sealant

A dermal-like equivalent is fashioned by electrospinning Types I and III collagen and elastin onto a rotating mandrel to form a sealant that mimics the architectural features and fiber characteristics of the native dermis. This type of sealant exhibits a deep, reticular-like layer composed of randomly arrayed, large diameter fibrils of Type I collagen and elastin. A more superficial layer is composed of small diameter fibrils of Type III collagen and elastin, preferentially deposited along a specific axis. Optionally, chondrotin-6-sulfate or other substances are combined with electroprocessing solutions or added after electroprocessing to further enhance the biological properties of the product. A computer controlled electrospinning device is used to deposit collagen and elastin into a dermis-like pattern of organization. The electrospinning device accommodates four separate electrospinning sources, one for the production of large-diameter fibrils of Type I collagen, one for the production of large-diameter fibrils of elastin, one for the production of small diameter fibrils of Type III collagen, and one for the production of small diameter fibrils of elastin. Electrospinning proceeds in a continuous fashion beginning exclusively with the ports that deliver the reticular layers and gradually shifting to the ports that fabricate the papillary layer. The layers are laid down in a continuum. To mimic the random orientation of the fibrils of the native reticular layer the electrospun reticular layer (large diameter Type I collagen and elastin) is deposited onto a rectangular mandrel rotating at less than 2000 rpm. Electrospinning onto a target rotating at this rate will produce a random array of fibrils. To produce fibrils on the order of 2-5 μm in diameter for the reticular layer of the dermal equivalent Type I collagen is electrospun from TFE solvent at a concentration of 100-120 mg/ml and elastin is electrospun from a concentration of 100-110 mg/ml of HFP. For the papillary layer, the target mandrel is rotated at 5500 rpm to preferentially array the fibrils along a common axis. To produce nanoscale fibers for the papillary layer, Type III collagen is electrospun from a solution containing a concentration of 60-80 mg/ml in TFE and elastin is electrospun from a solution containing a concentration of 70-80 mg/ml in HFP. Fibronectin or laminin or other substances are optionally added throughout the matrix. Optionally, electrospinning solutions are supplemented with varying concentrations of silver ions. Further, polyglycolic acid/polylactic acid polymer containing tetracycline is optionally electrosprayed as nanospheres. The nanospheres are distributed throughout the construct by electrospraying them onto the target as the construct is formed.

Optionally, the uppermost surface of an electrospun dermis is overlaid with closely packed fibrils or a continuous film of collagen prepared by extensive exposure to a crosslinking agent, to water vapor or to both. Subsequent to the formation of the film, additional layers of fibrils are optionally electrospun onto this structure to form the papillary and reticular layers. The resulting construct is composed of an electrospun matrix capped off with a continuous film of collagen.

Optionally, the lowermost surface of the electrospun dermis is underlaid with electroprocessed fibrinogen.

### Example 20

### Collagen Hemostat

A collagen hemostat was made by electrospinning Type I collagen from calf skin to make a nonwoven sheet of electroprocessed material. The collagen was electrospun from HFP containing 80 mg/ml collagen across a 5 inch air gap with 18-20 kV potential and rotation of approximately 3500 rpm or less) The electroprocessed material was about 300 μm thick, fluffy and soft to the touch. Mechanical testing of the electroprocessed material was conducted using “dog bone.” For this data, the electrospun scaffolds were subjected to stress-strain analysis using a INSTRON material testing device, Model No. 5543. The samples were trimmed into a “dog-bone” profile (FIG. 5) with offset ends to reduce grip effects and provide uniformity across samples. The testing was conducted with the tissue grips moving at a rate of 1 mm/min. The data acquisition rate was set to 20.0 Hz. The data integration and analysis was completed using INSTRON's MERLIN software. Results are presented in Table 3.

TABLE 3
Mechanical Properties of Collagen Hemostat

| Mechanical Property | Average Value |
|---|---|
| Maximum Stress (MPa) | 1.72 |
| Strain at Break (%) | 9.3 |
| Young's Modulus (MPa) | 4.7 |
| Max Load (N) | 0.15 |
| Break load (N) | 0.05 |

The matrix had an average pore size of between 5 and 6 μm² and an average fiber diameter of about 750 nm.

Adult Sprague Dawley Rats (500-700 g) were anesthetized with Ketaset (approximately 120 mg/kg). A mid-line incision was made in the abdominal wall, and the liver or spleen was exposed. Wounds on the liver were made using a razor blade to shave a large, shallow area from the liver. Injuries to the spleen were made by transection of the spleen with scissors. In both cases, the electroprocessed collagen material was applied after the injury with forceps directly to the wound surface. For some liver injuries, the wound was larger than the electroprocessed material, so additional pieces of the collagen mat were applied to cover the wound completely. Bleeding time was measured by visual inspection of the wound for blood flow. For both types of wounds, the collagen mat stopped bleeding completely in less than five seconds after application. No oozing or seeping from the wounds was observed.

### Example 21

### Physical Properties of Matrix Electrospun from Fibrinogen Solution

Lyophilized, bovine fibrinogen, Fraction I from plasma (Sigma-Aldrich Chemical Co.). was suspended in a solution composed of 9 parts HFP (Sigma-Aldrich Chemical Co.) and 1 part 10× minimal essential medium (MEM), Earle's (without L-glutamine and sodium bicarbonate) at a concentration of 0.167 grams/ml HFP/MEM. Once in solution or suspension, 2.5 ml of the fibrinogen solution was loaded into a 3.0 ml syringe. An 18-gauge stub (blunted) needle was then placed on the syringe to act as the electrospinning nozzle and charging point for the contained fibrinogen solution. The filled syringe was placed on a KD Scientific syringe pump using a Becton-Dickinson 1.0 ml Plunger set to dispense the solution at a rate of 1.85 ml/hr. The positive lead from the high voltage supply was attached to the metal stub of the syringe. The syringe pump was turned on and the high voltage supply was set at 22 kV. The grounded target was a 303 stainless steel mandrel (13.8 cm h×13.8 cm 1×0.5 cm w) placed four inches from the tip of the needle. The mandrel was rotated at approximately 3500 rpm. The fibrinogen solution was electrospun to form a white mat on the grounded mandrel. After electrospinning (0.4 ml total volume), the mat was removed from the mandrel and processed for scanning (SEM) and transmission (TEM) electron microscopy evaluation.

Uniaxial material testing was performed on a MTS Bionix 200 mechanical testing system incorporating a 100N load cell with an extension rate of 10.0 mm/minute to failure (MTS Systems Corp.; Eden Prairie, Minn.). The specimens were cut out of the mats using a “dog-bone” shaped template to assure uniformity and to isolate the failure point away from the grips. Tests were performed on dry and wet samples. Dry samples were tested in essentially the state they were found after electrospinning. Wet samples were soaked for approximately three hours in phosphate buffered saline. The specimens had a width of 2.75 mm and a gage length of 11.25 mm. The material properties chosen for comparison were the Young's modulus (tangential method), ultimate tensile strength (Peak Stress) and the strain to failure (% Strain at Break). Young's modulus and strain to failure were calculated automatically by the software). Results are presented in TABLE 4.

TABLE 4
Mechanical Property of Matrix Electrospun from Fibrinogen Solution

| Mechanical Property | Wetted Structure | Dry Structure |
|---|---|---|
| Young's Modulus (MPa) | 0.3 ± 0.05 | 84 ± 41 |
| Peak Stress (MPa) | 0.4 ± 0.05 | 2.3 ± 1.2 |
| Strain at Break (%) | 134 ± 18 | 8.3 ± 3.7 |

All patents, publications and abstracts cited herein are incorporated herein by reference in their entirety. These include, but are not limited to: (1) International (PCT) patent application “Engineered Muscle” PCT/US00/20974, filed Aug. 2, 2000, Publication No. WO 01/15754 A1; (2) International (PCT) patent application “Electroprocessed Fibrin-Based Matrices and Tissue” PCT US01/27409, filed Sep. 4, 2001, Publication No. WO 02/18441 A2; (3) International (PCT) patent application “Electroprocessed Collagen” serial number PCT/US01/43748, filed Nov. 16, 2001, Publication No. WO 02/40242 A1; (4) International (PCT) patent application “Electroprocessing in Drug Delivery and Cell Encapsulation” PCT/US01/32301, filed Oct. 18, 2001, Publication No. WO 02/32397A2; (5) U.S. patent application Ser. No. 10/447,670 filed May 28, 2003; and (6) U.S. patent application Ser. No. 10/409,682 filed Apr. 7, 2003. It should be understood that the foregoing relates only to preferred embodiments of the present invention and that numerous modifications or alterations can be made therein without departing from the spirit and the scope of the present invention as defined in the following claims.

### Example 21

### Electroprocessed Blends of Collagen and Synthetic Materials

A blend of 20:80 polydioxanone to collagen was electrospun into fibers. A solution of HFP containing The 80 mg/ml collagen, and 20 mg/ml polydioxanone. The experiment was repeated except that a copolymer of polycaprolactone:PLA (10PCL:90PLA) was substituted for the polydioxanone. The result in each case was a mat of electrospun fibers.

What is claimed is:

1. A composition comprising fibers of electroprocessed collagen or electroprocessed fibrinogen, wherein the fibers have an average diameter between about 50 nm and about 10 μm, and the composition is effective as a sealant.

2. A composition comprising isolated fibers comprising collagen or fibrinogen, wherein the fibers have an average diameter between about 50 nm and about 10 μm, and the composition is effective as a sealant.

3. A composition comprising electroprocessed fibrinogen, wherein the electroprocessed fibrinogen is insoluble in water.

4. A composition comprising fibrinogen, wherein the fibrinogen is insoluble in water.

5. A composition comprising electroprocessed fibrinogen, wherein the electroprocessed fibrinogen is present as fibers having a repeating banding pattern.

6. A composition comprising an electroprocessed material, wherein the electroprocessed material is effective as a sealant.

7. The composition of any of claims 1-6, wherein the composition is effective to cause hemostasis.

8. The composition of any of claims 1-7, further comprising a substance.

9. The composition claim 8, wherein the substance is selected from thrombin, aprotinin, Factor XIII, calcium chloride, hydroxyapatite, a fibrinolytic inhibitor, a fibrinolytic agent, fibronectin, or a combination thereof.

10. The use of the composition of any of claims 1-9 in the preparation of a sealant.

11. The use of the composition of any of claims 1-9 in the preparation of a medicament useful in providing physical reinforcement to tissue, repairing an injury or defect in tissue, promoting healing, causing hemostasis, or a combination thereof.

12. A method of manufacturing a composition, comprising electroprocessing one or more electrically-charged solutions comprising collagen, fibrinogen, or a combination thereof under conditions effective to electrodeposit electroprocessed material onto a substrate to form fibers having an average diameter between about 50 nm and about 10 μm.

13. A method of manufacturing the composition of any of claims 1-9, comprising electroprocessing one or more electrically-charged solutions comprising material under conditions effective to electrodeposit electroprocessed material onto a substrate to form the electroprocessed material.
